SEQUENCE LISTING (4 sequences)

SEQ ID NO: 1 - DNA, 699 bp, Spodoptera frugiperda
ctaatacgactcactatagggcaagcagtggtaacaacgcagagtacgcggggtcatttgctttcaaaaaccttcgagggagaccacggaagcaccaactacaaccttccgtagttactagtaaaagtgatccgtactttatttcacgtaaacgaaactcagttagctgctgaatttatttcgacgtgcagatttataaagttcgaaggatcacaatgaaaacgagcgcgtacaacgtgtacctgggagtcgtggccgccatgttggctctactgttcgtcacaattaatgctgcgccaatggaggcggacgatgagacggctgagaacaccctcgtggcgcatcccgatggtgacatggagctctcaggcccctgggatgctatcaacactgccgctctacgcaaactgctgctgcaacttgatgcagaggacaggatgggcggggtgacccgctcgtggccccaagctgagccccgcggttggggtctgcgggcgttggacagccgtctggcgcggcagtggagggcagacaagcggcaggtgcgattccgccagtgctacttcaacccaatttcctgcttccgcaagtgaaaacagcaccaactcaacgacgcatcgaccctttgacctagggtagcaagaacgaataaaacgtcgccataatctcccgaaaaaaaaaaaaaaaaaaaaaaaaaaa

SEQ ID NO: 2 - protein, 125 aa, Spodoptera frugiperda
MKTSAYNVYLGVVAAMLALLFVTINAAPMEADDETAENTLVAHPDGDMELSGPWDAINTAALRKLLLQLDAEDRMGGVTRSWPQAEPRGWGLRALDSRLARQWRADKRQVRFRQCYFNPISCFRK

SEQ ID NO: 3 - DNA, 307 bp, Spodoptera frugiperda
tagaatactcaagctatgcatccaacgcgttgggagctctcccatatggtcgacctgcaggcggccgcgaattcactagtgattctaatacgactcactatagggcaagcagtggtaaaacgcagagtacgcgggaacccaatttcctgcttccgcaagaagaagctgaaggattaaattattctatttctttcaattgcatagcattaccaatttaccatcttacattttgctattcatttaattaagatgacaaaattaataaaaatgtaacagccaaaaaaaaaaaaaaaaaaaaaaaaaaaaa

SEQ ID NO: 4 - protein, 53 aa, Spodoptera frugiperda
MHPTRWELSHMVDLQAAANSLVILIRLTIGQAVVKRRVRGNPISCFRKKKLKD

(19) United States
(12) Patent Application Publication
(10) Pub. No.: US 2019/0343122 A1
(43) Pub. Date: Nov. 14, 2019

(54) IMPROVED INSECT CONTROL STRATEGIES UTILIZING PHEROMONES AND RNAI

(71) Applicant: Provivi, Inc., Santa Monica, CA (US)

(72) Inventors: Pedro Coelho, Santa Monica, CA (US); Christopher Wheeler, Santa Monica, CA (US)

(21) Appl. No.: 16/304,762

(22) PCT Filed: May 26, 2017
(86) PCT No.: PCT/US17/34697

§ 371 Date: Nov. 27, 2018

Related U.S. Application Data
(60) Provisional application No. 62/342,807, filed on May 27, 2016.

(51) Int. Cl. A01N 49/00; A01N 57/16; A01N 25/00; A01N 27/00; C12N 15/82

(52) U.S. Cl. CPC A01N 49/00; A01N 57/16; C12N 15/8286; A01N 27/00; C12N 15/8218; A01N 25/006

(57) ABSTRACT

Systems and methods of preventing or reducing crop damage from pests are provided. In one embodiment, the method comprises: a) applying a mating disruption tactic to a field plot; and b) disrupting expression of one or more target genes in one or more pests, wherein crop damage is reduced in the field plot. In another embodiment, the method comprises applying an attract-and-kill tactic to a field plot, wherein said attract-and-kill tactic comprises: a) applying one or more semiochemicals or factors; and b) disrupting expression of one or more target genes in one or more pests, wherein said disruption is capable of killing the one or more pests, wherein crop damage is reduced in the field plot.

### CROSS-REFERENCE TO RELATED APPLICATIONS

This application claims the benefit of priority to U.S. Provisional Application No. 62/342,807, filed May 27, 2016, which is herein incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is PRVI-015_01WO_SeqList_ST25.txt. The text file is about 3.5 KB, was created on May 25, 2017, and is being submitted electronically via EFS-Web.

### FIELD OF THE INVENTION

The present disclosure relates to improved systems and methods for controlling pests. In one embodiment, the method comprises: a) applying a mating disruption tactic to a field plot; and b) disrupting expression of one or more target genes in one or more pests, wherein crop damage is reduced in the field plot. In another embodiment, the method comprises applying an attract-and-kill tactic to a field plot, wherein said attract-and-kill tactic comprises: a) applying one or more semiochemicals or factors; and b) disrupting expression of one or more target genes in one or more pests, wherein said disruption is capable of killing the one or more pests, wherein crop damage is reduced in the field plot.

### BACKGROUND OF THE INVENTION

About 10-16 percent of global crop production is lost to pests such as fungi, bacteria, viruses, insects, nematodes, viroids and oomycetes. About 67,000 different crop pest species—including plant pathogens, weeds, invertebrates and some vertebrate species—together cause about a 40 percent reduction in the world's crop yield. For example, the cotton bollworm, Helicoverpa zea, infests roughly 70% of the 3,800,000 acres of cotton grown in the US each year. Helicoverpa zea, also known as the corn earworm, is the second most important economic pest species in North America. H. zea migrates seasonally, mostly at night, and can be carried downwind up to 400 km. Furthermore, a strong relationship exists between increased global temperatures over the past 50 years and the expansion of crop pests.

Chemical pesticides have traditionally been used to control pests, but resistance of the pests to these chemicals has been increasing. More than 90 percent of the arthropod species with resistant populations are Diptera (35 percent), Lepidoptera (15 percent), Coleoptera (14 percent), Hemiptera (14 percent), or mites (14 percent). The heavy use of insecticides against disease-carrying mosquitoes has led to the disproportionately high number of resistant Diptera. Agricultural pests account for 59 percent of harmful resistant species, while medical and veterinary pests account for 41 percent. Statistical analyses suggest that for crop pests, resistance evolves most readily in those with an intermediate number of generations (four to ten) per year that feed either by chewing or by sucking on plant cell contents (Karaağaç SU: Insecticide resistance. In: Insecticides—Advances in Integrated Pest Management. Edited by Perveen F: In Tech; 2012:469-478). Additionally, broad-spectrum pesticides can adversely affect human health and the environment. They are often non-selective, harming beneficial organisms as well as pests. Thus there is a desire to employ safer and more environmentally friendly pest control techniques and limiting the amount of chemical pesticides.

Integrated pest management (IPM) considers all available pest control tactics and how these tactics fit with other agricultural practices to grow healthy crops and minimize the use of pesticides. The goal of IPM is to prevent pests from inflicting economic or aesthetic damage with the least risk to the environment. IPM involves the identification of pests, accurate measurement of pest populations, assessment of damage levels and knowledge of available pest management strategies or tactics that enable the specialist to make intelligent decisions about pest control. Pest control strategies can include chemical control; physical, mechanical and cultural controls; genetic control; and biological control.

Synthetic chemical pesticides can include inorganic substances like arsenic-containing salts or synthetic organic compounds like organophosphates, carbamates, and triazines. Pesticides such as insecticides can be classified according to shared chemical structures and modes of action (MoA). MoA is the specific process by which an insecticide kills an insect, or inhibits its growth. A good cultural practice is to use insecticides having different MoAs to slow the rate at which insects develop resistance to any one class of chemical insecticides.

Physical, mechanical and cultural controls include ecological landscaping to reduce field size and distance to habitats of natural enemies, erection of barriers, crop rotation, cover cropping, mechanical removal of pests (e.g., by hand or vacuums), improved crop residue management, better water management, and improved pest monitoring.

Genetic control strategies take advantage of naturally resistant plant or crop varieties, new plant or crop varieties bred for resistance, or transgenic plant or crop varieties. Genetic control strategies can also encompass production and release of sterile pests to prevent reproduction.

Biological control strategies encompass a number of non-chemical alternatives and usually include: macrobiological pesticides such as predators, parasites, and competitors that are released and spread on their own; microbial pesticides such as formulations of live or killed bacteria, viruses, fungi, protozoa and other microbes that are repeatedly applied to suppress pest populations; naturally sourced products and biochemicals such as peptides, nucleic acids and plant extracts; transgenic plants expressing plant protection compounds (plant incorporated protectants or PIPs); and pest behavior-modifying semiochemicals such as pheromones to trap pests or to suppress pest mating. Biological control strategies can minimize the impact on off-target and beneficial insects.

Some pest management techniques take advantage of the fact that the behaviors of pests are controlled by chemical signals emitted and detected amongst individuals. For example, male moths respond to calling females by detecting and following the female sex pheromone trail. Mating disruption (MD) is a pest management technique designed to control certain insect pests by introducing artificial stimuli, usually synthetic sex pheromones, that impair chemical communication between individuals causing the disruption of normal mate localization behavior and/or courtship, thus preventing mating and blocking the reproductive cycle. Mating disruption is advantageous in that the sex pheromones are species-specific, active in very small amounts and not known to be toxic to animals.

Thus, to ameliorate the use of chemical pesticides, combat pest resistance and adopt an integrated strategy to protect crops and other plants, new and more effective systems and methods of pest control taking advantage of combinatorial approaches and environmentally friendly technologies are needed. This disclosure provides such systems and methods.

### SUMMARY OF THE INVENTION

The present invention provides systems and methods for the control of pests, including insect pests which are plant and crop pests. The systems and methods of the present invention are useful in any plant culturing system, such as, but not limited to, those utilized in agronomy, horticulture, viticulture and arboriculture. The pest control systems and methods of the present invention find applications for plants grown in any situation, such as but not limited to plants grown in fields (e.g., large scale row crops), rangelands, forests, golf courses and nurseries.

In one embodiment of the present invention, systems and methods to control pests comprise a combination of mating disruption and disruption in expression of one or more target genes in one or more pests.

In some embodiments, the disrupting expression of one or more target genes in one or more pests in combination with a mating disruption tactic will lead to an additive effect on controlling the insect population. In other embodiments, the disrupting expression of one or more target genes in one or more pests in combination with a mating disruption tactic will lead to a synergistic effect on controlling the insect population. In aspects, the mating disruption tactic involves the use of a pheromone. In aspects, the disrupting expression of one or more target genes in one or more pests comprises disrupting by RNA interference (RNAi). Consequently, in aspects, the disclosure provides for additive effect combinations of pheromones and one or more RNAi-based insecticide, as well as synergistic effects between combinations of attractant pheromones and RNAi-based insecticide.

The present invention provides a method of reducing or preventing plant damage in a field plot which comprises plants of a plant population, wherein the field plot further comprises one or more pests capable of damaging the plants, said method comprising: a. applying a mating disruption tactic to the field plot, wherein said mating disruption tactic is capable of disrupting the mating of the one or more pests; and b. disrupting the expression of one or more target genes in the one or more pests, wherein said method reduces or prevents plant damage from the one or more pests as a result of the applications when compared to a control field plot which only had one or none of the applications. In some embodiments, applying a mating disruption tactic comprises applying one or more pheromones or pheromone blends. In other embodiments, the one or more pheromones or pheromone blends comprises one or more pheromones listed in Table 2. In some preferred embodiments, the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

In some embodiments, applying a mating disruption tactic comprises spraying one or more pheromones or pheromone blends in the field plot. In other embodiments, the one or more pheromones or pheromone blends comprises one or more pheromones listed in Table 2. In some preferred embodiments, the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

In some embodiments, applying a mating disruption tactic comprises emitting one or more pheromones or pheromone blends from one or more dispensers placed in the field plot. In other embodiments, the one or more pheromones or pheromone blends comprises one or more pheromones listed in Table 2. In some preferred embodiments, the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

In some embodiments, applying a mating disruption tactic comprises spraying one or more pheromones or pheromone blends in the field plot, and disrupting expression of one or more target genes comprises feeding dsRNA to the one or more pests. In a preferred embodiment, the dsRNA fed to the one or more pests are infused in phagostimulants. In some embodiments, applying a mating disruption tactic comprises spraying one or more pheromones or pheromone blends in the field plot, and disrupting expression of one or more target genes comprises spraying RNAi molecules in the field plot. In a preferred embodiment, the RNAi molecules are siRNA or dsRNA infused in phagostimulants. In some embodiments, applying a mating disruption tactic comprises scattering pheromone- or pheromone blend-coated granules in the field plot, and disrupting expression of one or more target genes comprises growing transgenic plants expressing RNAi molecules in the field plot as a source of food for the one or more pests.

In one embodiment, the target gene comprises one or more pheromone biosynthesis-activating neuropeptides (PBANs) in the one or more pests. In another embodiment, disrupting one or more PBANs makes the mating disruption more effective. In another embodiment, disrupting one or more PBANs comprises disrupting by RNA interference. In another embodiment, each PBAN is from a pest of the same species as each pest damaging the plants.

In some embodiments, the target gene comprises: chromatin-remodeling ATPases, prothoraciotropic hormone, molt-regulating transcription factors 3, eclosion hormone precursor, p450 monooxygenase, allatoregulating neuropeptides, 3-hydroxy-3-methylglutaryl coenzyme A reductase (HMGR), vacuolar-type H+-ATPases, chitinases, PCGP, arf1, arf2, tubulins, cullin-1, acetylcholine esterases, β1 integrins, iron-sulfur proteins, aminopeptidaseN, arginine kinases, chitin synthases, or any combination thereof, in the one or more pests.

In one embodiment, the one or more target genes comprises one or more genes associated with oviposition. In another embodiment, the genes associated with oviposition are selected from the group consisting of an allatoregulating neuropeptide, a GSK-3 gene, an EMP24/GP25 gene, a chemosensory protein gene, a subolesin/akirin transcription factor gene, an HMG-CoA reductase gene, a purity-of-essence gene, a glucose dehydrogenase gene, a neurocalcin homologue gene, a Scavenger receptor class B member 1 gene, an acyl-CoA delta-11-desaturase gene, a bcl-2-related ovarian killer gene, a ubiquinone biosynthesis gene and an odorant receptor gene.

In one embodiment, the mating disruption tactic is used to control one pest and the disruption in expression of one or more target genes is used to control another pest. In one embodiment, said mating disruption tactic is capable of disrupting the mating of a lepidopteran pest. In another embodiment, the target gene is from a sucking pest, such as a stink bug (pentatomid).

The present invention provides a method of reducing or preventing plant damage in a field plot which comprises plants of a plant population, wherein the field plot further comprises one or more pests capable of damaging the plants, said method comprising applying an attract-and-kill tactic to the field plot, wherein said attract-and-kill tactic comprises: applying one or more semiochemicals or factors; and disrupting expression of one or more target genes in one or more pests, wherein said disruption is capable of killing the one or more pests, wherein said method reduces or prevents plant damage from the one or more pests as a result of the application when compared to a control field plot which did not have the application.

In one embodiment of the present invention, systems and methods to control pests comprise applying an attract-and-kill tactic. In one embodiment, applying an attract-and-kill tactic comprises applying one or more semiochemicals or factors and disrupting expression of a target gene in one or more pests. In one embodiment, the disruption in expression of the target gene injures or kills the pest. In another embodiment, the disruption in expression of one or more target genes comprises RNAi. In another embodiment, the one or more pests is a sucking pest. In another embodiment, the one or more target genes comprises one or more genes associated with lethality or reduced growth when the gene is down regulated. In another embodiment, the genes associated with lethality or reduced growth when down regulated are selected from the group consisting of a chitinase gene, a cytochrome P450 monooxygenase gene, a vacuolar-type Ht ATPase gene, a chromatin remodelling ATPase gene, a prothoraciotropic hormone gene, a molt-regulating transcription factors 3 gene, a eclosion hormone precursor gene, a chitin synthase gene, PGCP gene, a tubulin gene, an arf gene, a trehalose phosphate synthase gene, a ribosomal protein gene, a beta-actin gene, a protein transport gene, a coatomer subunit gene, a cullin gene, a chitinase gene, an acetylcholinesterase gene, a β1 integrin gene, an iron-sulfur protein gene, an aminopeptidaseN gene, an arginine kinase gene and a proteasome-associated gene.

In another embodiment, the one or more semiochemicals or factors comprise one or more attractants. In another embodiment, the one or more attractants comprise one or more host plant chemical, non-host plant chemical, synthetic volatile chemical, or natural volatile chemical. In another embodiment, the one or more attractants are identified through binding to one or more pest odorant binding proteins. In another embodiment, the one or more attractants comprise one or more host plant volatile chemical. In another embodiment, the one or more host plant volatile chemical comprise heptanal or benzaldehyde. In another embodiment, the one or more attractants comprise one or more male pheromones. In another embodiment, the one or more attractants comprise one or more ovipositioning pheromones. In another embodiment, the one or more attractants comprise one or more female attractants. In another embodiment, the one or more female attractants comprise ethylene. In another embodiment, the one or more attractants comprise one or more kairomones. In some embodiments, the one or more attractants comprise one or more pheromones or pheromone blends. In other embodiments, the one or more pheromones or pheromone blends comprises one or more pheromones listed in Table 2. In some preferred embodiments, the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

In some embodiments, applying one or more semiochemicals or factors comprises emitting one or more pheromones or pheromone blends from one or more dispensers placed in one or more traps in the field plot. In other embodiments, the one or more pheromones or pheromone blends comprises one or more pheromones listed in Table 2. In some preferred embodiments, the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof. In some embodiments, applying one or more semiochemicals or factors comprises emitting one or more pheromones or pheromone blends from one or more dispensers placed in one or more traps in the field plot, and wherein disrupting expression of one or more target genes comprises feeding dsRNA to the one or more pests.

In some embodiments, disrupting the expression of one or more target genes in the one or more pests comprises RNA interference (RNAi). In further embodiments, the RNAi comprises one or more double-stranded RNA, one or more small interfering RNA (siRNA), or a combination thereof. In some embodiments, the one or more double-stranded RNA, one or more small interfering RNA (siRNA), or a combination thereof, are expressed in a plant. In other embodiments, the one or more double-stranded RNA, one or more small interfering RNA (siRNA), or a combination thereof, are formulated for a broadcast spray, a feeding station, a food trap, or any combination thereof.

In some embodiments, the one or more pests comprises one or more sucking pests. In some embodiments, the one or more pests is a member of the class Insecta. In further embodiments, the one or more pests is a member of the order Lepidoptera. In some embodiments, the one or more pests is a member of the order Hemiptera. In some embodiments, the one or more pests is a member of the family Noctuidae. In further embodiments, the one or more pests is a member of the family Pentatomidae. In some embodiments, the one or more pests is a member of the order Coleoptera. In further embodiments, the one or more pests is a member of the family Curculionidae. In some embodiments, the one or more pests is a member of a genus selected from the group consisting of: Helicoverpa, Spodoptera, Euschistus, Anthonomus and Nezara, or any combination thereof. In further embodiments, the one or more pests is a species selected from the group consisting of: Helicoverpa zea, Helicoverpa armigera, Spodoptera frupperda, Spodoptera cosmioides, Euschistus heros, Anthonomus grandis and Nezara viridula, or any combination thereof.

### BRIEF DESCRIPTION OF THE DRAWINGS

FIG. 1 shows nucleotide (SEQ ID NO: 1) and the deduced amino acid (SEQ ID NO: 2) sequences of the S. frugiperda AS cDNA. The sequences are numbered at the right. The amino acid sequence of the Spofr-AS is shown in bold type. Possible dibasic proteolytic cleavage sites are in boxes. The possible site for cleavage of the signal sequence is marked with a downward arrow. The potential polyadenylation signal is shown in bold type and underlined; --- represents the stop codon. From Abdel-latief et al. 2003. Molecular characterization of cDNAs from the fall armyworm Spodoptera frugiperda encoding Manduca sexta allatotropin and allatostatin preprohormone peptides. Insect Biochemistry and Molecular Biology 33: 467-476.

FIG. 2 shows the nucleotide (SEQ ID NO: 3) and the deduced amino acid (SEQ ID NO: 4) sequences of the Spofr-AT 2 cDNA. The sequences are numbered at the right. The Spofr-AT 2 amino acid sequence is shown in bold type. Potential cleavage sites are in boxes. The polyadenylation signal is shown in bold type and is underlined; --- represents the stop codon. A possible signal peptide cleavage site is marked with a downward arrow. From Abdel-latief et al. 2004. Characterization of a novel peptide with allatotropic activity in the fall armyworm Spodoptera frugiperda. Regulatory Peptides 122: 69-78.

### DETAILED DESCRIPTION OF THE INVENTION

All publications, patents and patent applications, including any drawings and appendices, herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

In the description and tables which follow, a number of terms are used. In order to provide a clear and consistent understanding of the specification and claims, including the scope to be given such terms, the following definitions are provided:

As used herein, the term “a” refers to a noun and can refer to the singular or the plural version. Thus, a reference to a pheromone can refer to one pheromone or more than one pheromone.

As used herein, “consisting essentially of” refers to a composition “consisting essentially of” certain elements is limited to the inclusion of those elements, as well as to those elements that do not materially affect the basic and novel characteristics of the inventive composition.

As used herein, the terms “comprises,” “comprising,” “includes,” “including,” “has,” “having, “contains,” “containing,” or any other variation thereof, are intended to cover a non-exclusive inclusion. A composition, mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive “or” and not to an exclusive “or.”

As used herein, the term “about” in reference to a numerical value refers to the range of values somewhat lesser or greater than the stated value, as understood by one of skill in the art. For example, the term “about” could mean a value ranging from plus or minus a percentage (e.g., ±1%, ±2%, ±5%, or ±10%) of the stated value. Furthermore, since all numbers, values, and expressions referring to quantities used herein are subject to the various uncertainties of measurement encountered in the art, then unless otherwise indicated, all presented values may be understood as modified by the term “about.”

As used herein, the term “plant” refers to any living organism belonging to the kingdom Plantae (i.e., any genus/species in the Plant Kingdom).

As used herein, the term “monocotyledon” or “monocot” refer to any of a subclass (Monocotyledoneae) of flowering plants having an embryo containing only one seed leaf and usually having parallel-veined leaves, flower parts in multiples of three, and no secondary growth in stems and roots. Examples include lilies; orchids; rice; corn, grasses, such as tall fescue, goat grass, and Kentucky bluegrass; grains, such as wheat, oats and barley; irises; onions and palms.

As used herein, the terms “dicotyledon” and “dicot” refer to a flowering plant having an embryo containing two seed halves or cotyledons. Examples include tobacco; tomato; the legumes, including peas, alfalfa, clover and soybeans; oaks; maples; roses; mints; squashes; daisies; walnuts; cacti; violets and buttercups.

As used herein, the term “population” means a genetically homogeneous or heterogeneous collection of plants sharing a common genetic derivation.

As used herein, the term “phenotype” refers to the observable characters of an individual cell, cell culture, organism (e.g., a plant), or group of organisms which results from the interaction between that individual's genetic makeup (i.e., genotype) and the environment.

As used herein, the term “variety” or “cultivar” means a group of similar plants that by structural features and performance can be identified from other varieties within the same species. The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

As used herein, the term “genotype” refers to the genetic makeup of an individual cell, cell culture, tissue, organism (e.g., a plant), or group of organisms.

As used herein, the term “hybrid” refers to any individual cell, tissue or plant resulting from a cross between parents that differ in one or more genes.

As used herein, the term “inbred” or “inbred line” refers to a relatively true-breeding strain.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

As used herein, the term “plant part” refers to any part of a plant including but not limited to the shoot, root, stem, seeds, fruits, stipules, leaves, petals, flowers, ovules, bracts, branches, petioles, internodes, bark, pubescence, tillers, rhizomes, fronds, blades, pollen, stamen, rootstock, scion and the like. The two main parts of plants grown in some sort of media, such as soil, are often referred to as the “above-ground” part, also often referred to as the “shoots”, and the “below-ground” part, also often referred to as the “roots”.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the terms “polypeptide,” “peptide,” and “protein” are used interchangeably herein to refer to polymers of amino acids of any length. These terms also include proteins that are post-translationally modified through reactions that include glycosylation, acetylation and phosphorylation.

As used herein, “attract-and-kill” refers to a technique or tactic in pest management where one or more semiochemicals or factors and one or more killing or sterilizing agents are applied in a concentrated area at the pest source to provide pest control. In one embodiment, the one or more semiochemicals comprise attractants or crude baits. In another embodiment, the one or more semiochemicals or factors comprise one or more phagostimulants. In one embodiment, the one or more semiochemicals comprise one or more pheromones or pheromone blends. In another embodiment, the one or more factors comprise factors that stimulate earlier egg maturation/oogenesis and/or ovipositioning. In one embodiment the factors that stimulate earlier egg maturation/oogenesis and/or ovipositioning are oogenesis and oviposition factors (OOSFs). In another embodiment, the OOSFs are from crude extracts of male accessory glands (MAG). In another embodiment, the OOSFs are purified by fractionation or sub-fractionation from crude extracts of male accessory glands (MAG). In one embodiment, the killing agent can comprise an insecticide or pesticide. In another embodiment, the insecticide or pesticide can comprise a biological insecticide or pesticide, a chemical insecticide or pesticide, a plant incorporated insecticide or pesticide, or any combination thereof. In one embodiment, the insecticide or pesticide is an RNAi-based insecticide or RNAi-based pesticide. In another embodiment, “attract-and-kill” can refer to “attract-and-RNAi-kill” when the killing agent is an RNAi-based insecticide or pesticide. In one embodiment, the pest can be lured to a pest control device which comprises a substance that can quickly or eventually kill the pest, e.g., a pesticide, poison, biological agent, etc. In one embodiment, a segment of a capsule can contain a substance (e.g., an adhesive, powder, coating, etc.) that contains a contact pesticide that kills an insect that contacts the substance. The pesticide could work by any mechanism, such as by poison, e.g., a stomach poison, a biological agent such as Codling moth granulosis virus, a Molt accelerator, diatomaceous earth, or any other kind of ingestible poison. In another embodiment, semiochemical attractants used to lure the pest can be chemical signals, visual cues, acoustic cues, or a combination of any of these signals and cues. This pest management technique is also known as lure and kill.

As used herein, “attractant” refers to a natural or synthetic agent that attracts or lures, for example, animals, insects, birds, etc. Attractants can include: sexual attractants which affect mating behavior; food attractants; attractants that affect egg-laying, or ovipositioning.

As used herein, “repellent” or “deterrent” refers to a substance applied to a surface which discourages pests from landing or climbing on that surface. In one embodiment, the surface can be a whole plant or plant part.

As used herein, a “dispenser” or “dispensing device” refers to an automated device that provides a pheromone reservoir and a controlled release of the content. Examples of the controlled release include, but not limited to, atomize, dispense, diffuse, evaporate, spray, vaporize, or the like. The rate of controlled release may be continuous, periodic, or timed intervals.

As used herein, “highly dispersive insect”, “highly dispersive insect pest” or “highly dispersive pest” refers to any pest that cannot be controlled by mating disruption over an area less about four hectares. Highly dispersive insect pests are difficult to control via mating disruption at small scales, usually due to the immigration of gravid females. Mating disruption for these types of pests is more effective with an area-wide management program.

As used herein, “host”, “host plant” or “host crop” refers to a crop or plant that a given pest feeds or otherwise subsists upon. As used herein, “non-host”, “non-host plant” or “non-host crop” refers to a crop or plant that a given pest usually does not feed or otherwise subsist upon under normal field conditions.

As used herein, “insecticide” refers to pesticides that are formulated to kill, harm, repel or mitigate one or more species of insect. Insecticides can be of chemical or biological origin. Insecticides include peptides, proteins and nucleic acids such as double-stranded DNA, single-stranded DNA, double-stranded RNA, single-stranded RNA and hairpin DNA or RNA. Examples of peptide insecticides include Spear™-T for the treatment of thrips in vegetables and ornamentals in greenhouses, Spear™-P to control the Colorado Potato Beetle, and Spear™-C to protect crops from lepidopteran pests (Vestaron Corporation, Kalamazoo, Mich.). Insecticides can be viruses such as Gemstar® (Certis USA) that kills larvae of Heliothis and Helicoverpa species. Insecticides can be packaged in various forms including sprays, dusts, gels, and baits. Insecticides can work through different modes of action (MoAs). Table 1 lists insecticides associated with various MoAs and Table 1a is a list of exemplary pesticides.

TABLE 1
Exemplary insecticides associated with various modes of action

| | | | Physiological |
|---|---|---|---|
| | | | function(s) |
| Mode of Action | Compound class | Exemplary insecticides | affected |
| acetylcholinesterase | carbamates | Alanycarb, Aldicarb, | Nerve and |
| (AChE) inhibitors | | Bendiocarb, Benfuracarb, | muscle |
| | | Butocarboxim, | |
| | | Butoxycarboxim, Carbaryl, | |
| | | Carbofuran, Carbosulfan, | |
| | | Ethiofencarb, Fenobucarb, | |
| | | Formetanate, Furathiocarb, | |
| | | Isoprocarb, Methiocarb, | |
| | | Methomyl, Metolcarb, | |
| | | Oxamyl, Pirimicarb, Propoxur, | |
| | | Thiodicarb, Thiofanox, | |
| | | Triazamate, Trimethacarb, | |
| | | XMC, Xylylcarb | |
| acetylcholinesterase | organophosphates | Acephate, Azamethiphos, | Nerve and |
| (AChE) inhibitors | | Azinphos-ethyl, Azinphos- | muscle |
| | | methyl, Cadusafos, | |
| | | Chlorethoxyfos, | |
| | | Chlorfenvinphos, | |
| | | Chlormephos, Chlorpyrifos, | |
| | | Chlorpyrifos-methyl, | |
| | | Coumaphos, Cyanophos, | |
| | | Demeton-S-methyl, Diazinon, | |
| | | Dichlorvos/DDVP, | |
| | | Dicrotophos, Dimethoate, | |
| | | Dimethylvinphos, Disulfoton, | |
| | | EPN, Ethion, Ethoprophos, | |
| | | Famphur, Fenamiphos, | |
| | | Fenitrothion, Fenthion, | |
| | | Fosthiazate, Heptenophos, | |
| | | Imicyafos, Isofenphos, | |
| | | Isopropyl O- | |
| | | (methoxyaminothio- | |
| | | phosphoryl) salicylate, | |
| | | Isoxathion, Malathion, | |
| | | Mecarbam, Methamidophos, | |
| | | Methidathion, Mevinphos, | |
| | | Monocrotophos, Naled, | |
| | | Omethoate, Oxydemeton- | |
| | | methyl, Parathion, Parathion- | |
| | | methyl, Phenthoate, Phorate, | |
| | | Phosalone, Phosmet, | |
| | | Phosphamidon, Phoxim, | |
| | | Pirimiphos-methyl, | |
| | | Profenofos, Propetamphos, | |
| | | Prothiofos, Pyraclofos, | |
| | | Pyridaphenthion, Quinalphos, | |
| | | Sulfotep, Tebupirimfos, | |
| | | Temephos, Terbufos, | |
| | | Tetrachlorvinphos, | |
| | | Thiometon, Triazophos, | |
| | | Trichlorfon, Vamidothion | |
| GABA-gated | cyclodiene | Chlordane, Endosulfan | Nerve and |
| chloride channel | organochlorines | | muscle |
| blockers | | | |
| GABA-gated | phenylpyrazoles | Ethiprole, Fipronil | Nerve and |
| chloride channel | (Fiproles) | | muscle |
| blockers | | | |
| sodium channel | pyrethroids, | Acrinathrin, Allethrin, | Nerve and |
| modulators | pyrethrins | Bifenthrin, Bioallethrin, | muscle |
| | | Bioallethrin S-cyclopentenyl, | |
| | | Bioresmethrin, Cycloprothrin, | |
| | | Cyfluthrin, Cyhalothrin, | |
| | | Cypermethrin, Cyphenothrin | |
| | | [(1R)-trans- isomers], | |
| | | Deltamethrin, Empenthrin | |
| | | [(EZ)- (1R)- isomers], | |
| | | Esfenvalerate, Etofenprox, | |
| | | Fenpropathrin, Fenvalerate, | |
| | | Flucythrinate, Flumethrin, | |
| | | Halfenprox, Kadathrin, | |
| | | Phenothrin [(1R)-trans- | |
| | | isomer], Prallethrin, Pyrethrins | |
| | | (pyrethrum), Resmethrin, | |
| | | Silafluofen, Tefluthrin, | |
| | | Tetramethrin, Tetramethrin | |
| | | [(1R)- isomers], Tralomethrin, | |
| | | Transfluthrin, alpha- | |
| | | Cypermethrin, beta-Cyfluthrin, | |
| | | beta-Cypermethrin, d-cis-trans | |
| | | Allethrin, d-trans Allethrin, | |
| | | gamma-Cyhalothrin, lambda- | |
| | | Cyhalothrin, tau-Fluvalinate, | |
| | | theta-Cypermethrin, zeta- | |
| | | Cypermethrin | |
| sodium channel | DDT, | DDT, methoxychlor | Nerve and |
| modulators | methoxychlor | | muscle |
| nicotinic | neonicotinoids | Acetamiprid, Clothianidin, | Nerve and |
| acetylcholine | | Dinotefuran, Imidacloprid, | muscle |
| receptor (nAChR) | | Nitenpyram, Thiacloprid, | |
| competitive | | Thiamethoxam | |
| modulators | | | |
| nicotinic | nicotine | nicotine | Nerve and |
| acetylcholine | | | muscle |
| receptor (nAChR) | | | |
| competitive | | | |
| modulators | | | |
| nicotinic | sulfoximines | sulfoxaflor | Nerve and |
| acetylcholine | | | muscle |
| receptor (nAChR) | | | |
| competitive | | | |
| modulators | | | |
| nicotinic | butenolides | Flupyradifurone | Nerve and |
| acetylcholine | | | muscle |
| receptor (nAChR) | | | |
| competitive | | | |
| modulators | | | |
| nicotinic | spinosyns | Spinetoram, Spinosad | Nerve and |
| acetylcholine | | | muscle |
| receptor (nAChR) | | | |
| allosteric modulators | | | |
| Glutamate-gated | avermectins, | Abamectin, Emamectin | Nerve and |
| chloride channel | milbemycins | benzoate, Lepimectin, | muscle |
| (GluCl) allosteric | | Milbemectin | |
| modulators | | | |
| juvenile hormone | juvenile hormone | Hydroprene, Kinoprene, | Growth |
| mimics | analogues | Methoprene | |
| juvenile hormone | Fenoxycarb | Fenoxycarb | Growth |
| mimics | | | |
| juvenile hormone | Pyriproxyfen | Pyriproxyfen | Growth |
| mimics | | | |
| miscellaneous non- | alkyl halides | Methyl bromide and other | Unknown or |
| specific (multi-site) | | alkyl halides | non-specific |
| inhibitors | | | |
| miscellaneous non- | Chloropicrin | Chloropicrin | Unknown or |
| specific (multi-site) | | | non-specific |
| inhibitors | | | |
| miscellaneous non- | fluorides | Cryolite, sulfuryl fluoride | Unknown or |
| specific (multi-site) | | | non-specific |
| inhibitors | | | |
| miscellaneous non- | borates | Borax, Boric acid, Disodium | Unknown or |
| specific (multi-site) | | octaborate, Sodium borate, | non-specific |
| inhibitors | | Sodium metaborate | |
| miscellaneous non- | tartar emetic | tartar emetic | Unknown or |
| specific (multi-site) | | | non-specific |
| inhibitors | | | |
| miscellaneous non- | methyl | Dazomet, Metam | Unknown or |
| specific (multi-site) | isothiocyanate | | non-specific |
| inhibitors | generators | | |
| modulators of | Pyridine | Pymetrozine, Pyrifluquinazon | Nerve and |
| chordotonal organs | azomethine | | muscle |
| | derivatives | | |
| mite growth | Clofentezine, | Clofentezine, Diflovidazin, | Growth |
| inhibitors | Diflovidazin, | Hexythiazox | |
| | Hexythiazox | | |
| mite growth | Etoxazole | Etoxazole | Growth |
| inhibitors | | | |
| microbial disruptors | Bacillus | Bt var. aizawai, Bt var. | Midgut |
| of insect midgut | thuringiensis and | israelensis, Bt var. kurstaki, Bt | |
| membranes | the insecticidal | var. tenebrionensis | |
| | proteins they | | |
| | produce | | |
| microbial disruptors | Bacillus | Bacillus sphaericus | Midgut |
| of insect midgut | sphaericus | | |
| membranes | | | |
| inhibitors of | Diafenthiuron | Diafenthiuron | Respiration |
| mitochondrial ATP | | | |
| synthase | | | |
| inhibitors of | organotin miticides | Azocyclotin, Cyhexatin, | Respiration |
| mitochondrial ATP | | Fenbutatin oxide | |
| synthase | | | |
| inhibitors of | Propargite | Propargite | Respiration |
| mitochondrial ATP | | | |
| synthase | | | |
| inhibitors of | Tetradifon | Tetradifon | Respiration |
| mitochondrial ATP | | | |
| synthase | | | |
| uncouplers of | Chlorfenapyr, | Chlorfenapyr, DNOC, | Respiration |
| oxidative | DNOC, Sulfuramid | Sulfuramid | |
| phosphorylation via | | | |
| disruption of the | | | |
| proton gradient | | | |
| Nicotinic | nereistoxin | Bensultap, Cartap | Nerve and |
| acetylcholine | analogues | hydrochloride, Thiocyclam, | muscle |
| receptor (nAChR) | | Thiosultap-sodium | |
| channel blockers | | | |
| inhibitors of chitin | benzoylureas | Bistrifluron, Chlorfluazuron, | Growth |
| biosynthesis, type 0 | | Diflubenzuron, Flucycloxuron, | |
| | | Flufenoxuron, Hexaflumuron, | |
| | | Lufenuron, Novaluron, | |
| | | Noviflumuron, Teflubenzuron, | |
| | | Triflumuron | |
| inhibitors of chitin | Buprofezin | Buprofezin | Growth |
| biosynthesis, type 1 | | | |
| moulting disruptor, | Cyromazine | Cyromazine | Growth |
| Dipteran | | | |
| ecdysone receptor | diacylhydrazines | Chromafenozide, | Growth |
| agonists | | Halofenozide, | |
| | | Methoxyfenozide, | |
| | | Tebufenozide | |
| octopamine receptor | Amitraz | Amitraz | Nerve and |
| agonists | | | muscle |
| mitochondrial | Hydramethylnon | Hydramethylnon | Respiration |
| complex III electron | | | |
| transport inhibitors | | | |
| mitochondrial | Acequinocyl | Acequinocyl | Respiration |
| complex III electron | | | |
| transport inhibitors | | | |
| mitochondrial | Fluacrypyrim | Fluacrypyrim | Respiration |
| complex III electron | | | |
| transport inhibitors | | | |
| mitochondrial | Bifenazate | Bifenazate | Respiration |
| complex III electron | | | |
| transport inhibitors | | | |
| mitochondrial | Meti acaricides and | Fenazaquin, Fenpyroximate, | Respiration |
| complex I electron | insecticides | Pyridaben, Pyrimidifen, | |
| transport inhibitors | | Tebufenpyrad, Tolfenpyrad | |
| mitochondrial | Rotenone | Rotenone | Respiration |
| complex I electron | | | |
| transport inhibitors | | | |
| voltage-dependent | oxadiazines | Indoxacarb | Nerve and |
| sodium channel | | | muscle |
| blockers | | | |
| voltage-dependent | semicarbazones | Metaflumizone | Nerve and |
| sodium channel | | | muscle |
| blockers | | | |
| inhibitors of acetyl | tetronic and | Spirodiclofen, Spiromesifen, | Growth |
| CoA carboxylase | tetramic acid | Spirotetramat | |
| | derivatives | | |
| mitochondrial | phosphides | Aluminium phosphide, | Respiration |
| complex IV electron | | Calcium phosphide, | |
| transport inhibitors | | Phosphine, Zinc phosphide | |
| mitochondrial | cyanides | Calcium cyanide, Potassium | Respiration |
| complex IV electron | | cyanide, Sodium cyanide | |
| transport inhibitors | | | |
| mitochondrial | beta-ketonitrile | Cyenopyrafen, Cyflumetofen | Respiration |
| complex II electron | derivatives | | |
| transport inhibitors | | | |
| mitochondrial | carboxanilides | Pyflubumide | Respiration |
| complex II electron | | | |
| transport inhibitors | | | |
| ryanodine receptor | diamides | Chlorantraniliprole, | Nerve and |
| modulators | | Cyantraniliprole, | muscle |
| | | Flubendiamide | |
| Chordotonal organ | Flonicamid | Flonicamid | Nerve and |
| modulators - | | | muscle |
| undefined target site | | | |
| compounds of | Azadirachtin | Azadirachtin | Unknown |
| unknown or uncertain | | | |
| mode of action | | | |
| compounds of | Benzoximate | Benzoximate | Unknown |
| unknown or uncertain | | | |
| mode of action | | | |
| compounds of | Bromopropylate | Bromopropylate | Unknown |
| unknown or uncertain | | | |
| mode of action | | | |
| compounds of | Chinomethionat | Chinomethionat | Unknown |
| unknown or uncertain | | | |
| mode of action | | | |
| compounds of | Dicofol | Dicofol | Unknown |
| unknown or uncertain | | | |
| mode of action | | | |
| compounds of | lime sulfur | lime sulfur | Unknown |
| unknown or uncertain | | | |
| mode of action | | | |
| compounds of | Pyridalyl | Pyridalyl | Unknown |
| unknown or uncertain | | | |
| mode of action | | | |
| compounds of | sulfur | sulfur | Unknown |
| unknown or uncertain | | | |
| mode of action | | | |

Adapted from www.irac - online.org

TABLE 1a
Exemplary list of pesticides

| Category | Compounds |
|---|---|
| INSECTICIDES | |
| arsenical insecticides | calcium arsenate |
| | copper acetoarsenite |
| | copper arsenate |
| | lead arsenate |
| | potassium arsenite |
| | sodium arsenite |
| botanical insecticides | allicin |
| | anabasine |
| | azadirachtin |
| | carvacrol |
| | d-limonene |
| | matrine |
| | nicotine |
| | nornicotine |
| | oxymatrine |
| | pyrethrins |
| | cinerins |
| | cinerin I |
| | cinerin II |
| | jasmolin I |
| | jasmolin II |
| | pyrethrin I |
| | pyrethrin II |
| | quassia |
| | rhodojaponin-III |
| | rotenone |
| | ryania |
| | sabadilla |
| | sanguinarine |
| | triptolide |
| carbamate insecticides | bendiocarb |
| | carbaryl |
| benzofuranyl methylcarbamate | benfuracarb |
| insecticides | carbofuran |
| | carbosulfan |
| | decarbofuran |
| | furathiocarb |
| dimethylcarbamate insecticides | dimetan |
| | dimetilan |
| | hyquincarb |
| | isolan |
| | pirimicarb |
| | pyramat |
| | pyrolan |
| oxime carbamate insecticides | alanycarb |
| | aldicarb |
| | aldoxycarb |
| | butocarboxim |
| | butoxycarboxim |
| | methomyl |
| | nitrilacarb |
| | oxamyl |
| | tazimcarb |
| | thiocarboxime |
| | thiodicarb |
| | thiofanox |
| phenyl methylcarbamate insecticides | allyxycarb |
| | aminocarb |
| | bufencarb |
| | butacarb |
| | carbanolate |
| | cloethocarb |
| | CPMC |
| | dicresyl |
| | dimethacarb |
| | dioxacarb |
| | EMPC |
| | ethiofencarb |
| | fenethacarb |
| | fenobucarb |
| | isoprocarb |
| | methiocarb |
| | metolcarb |
| | mexacarbate |
| | promacyl |
| | promecarb |
| | propoxur |
| | trimethacarb |
| | XMC |
| | xylylcarb |
| diamide insecticides | broflanilide |
| | chlorantraniliprole |
| | cyantraniliprole |
| | cyclaniliprole |
| | cyhalodiamide |
| | flubendiamide |
| | tetraniliprole |
| dinitrophenol insecticides | dinex |
| | dinoprop |
| | dinosam |
| | DNOC |
| fluorine insecticides | barium hexafluorosilicate |
| | cryolite |
| | flursulamid |
| | sodium fluoride |
| | sodium hexafluorosilicate |
| | sulfluramid |
| formamidine insecticides | amitraz |
| | chlordimeform |
| | formetanate |
| | formparanate |
| | medimeform |
| | semiamitraz |
| fumigant insecticides | acrylonitrile |
| | carbon disulfide |
| | carbon tetrachloride |
| | carbonyl sulfide |
| | chloroform |
| | chloropicrin |
| | cyanogen |
| | para-dichlorobenzene |
| | 1,2-dichloropropane |
| | dithioether |
| | ethyl formate |
| | ethylene dibromide |
| | ethylene dichloride |
| | ethylene oxide |
| | hydrogen cyanide |
| | methyl bromide |
| | methyl iodide |
| | methylchloroform |
| | methylene chloride |
| | naphthalene |
| | phosphine |
| | sodium tetrathiocarbonate |
| | sulfuryl fluoride |
| | tetrachloroethane |
| inorganic insecticides | borax |
| | boric acid |
| | calcium polysulfide |
| | copper oleate |
| | diatomaceous earth |
| | mercurous chloride |
| | potassium thiocyanate |
| | silica gel |
| | sodium thiocyanate |
| insect growth regulators | |
| chitin synthesis inhibitors | buprofezin |
| | cyromazine |
| benzoylphenylurea chitin synthesis | bistrifluron |
| inhibitors | chlorbenzuron |
| | chlorfluazuron |
| | dichlorbenzuron |
| | diflubenzuron |
| | flucycloxuron |
| | flufenoxuron |
| | hexaflumuron |
| | lufenuron |
| | novaluron |
| | noviflumuron |
| | penfluron |
| | teflubenzuron |
| | triflumuron |
| juvenile hormone mimics | dayoutong |
| | epofenonane |
| | fenoxycarb |
| | hydroprene |
| | kinoprene |
| | methoprene |
| | pyriproxyfen |
| | triprene |
| juvenile hormones | juvenile hormone I |
| | juvenile hormone II |
| | juvenile hormone III |
| moulting hormone agonists | chromafenozide |
| | furan tebufenozide |
| | halofenozide |
| | methoxyfenozide |
| | tebufenozide |
| | yishijing |
| moulting hormones | α-ecdysone |
| | ecdysterone |
| moulting inhibitors | diofenolan |
| precocenes | precocene I |
| | precocene II |
| | precocene III |
| unclassified insect growth regulators | dicyclanil |
| macrocyclic lactone insecticides | |
| avermectin insecticides | abamectin |
| | doramectin |
| | emamectin |
| | eprinomectin |
| | ivermectin |
| | selamectin |
| milbemycin insecticides | lepimectin |
| | milbemectin |
| | milbemycin oxime |
| | moxidectin |
| spinosyn insecticides | spinetoram |
| | spinosad |
| neonicotinoid insecticides | |
| nitroguanidine neonicotinoid | clothianidin |
| insecticides | dinotefuran |
| | imidacloprid |
| | imidaclothiz |
| | thiamethoxam |
| nitromethylene neonicotinoid | nitenpyram |
| insecticides | nithiazine |
| pyridylmethylamine neonicotinoid | acetamiprid |
| insecticides | imidacloprid |
| | nitenpyram |
| | paichongding |
| | thiacloprid |
| nereistoxin analogue insecticides | bensultap |
| | cartap |
| | polythialan |
| | thiocyclam |
| | thiosultap |
| organochlorine insecticides | bromo-DDT |
| | camphechlor |
| | DDT |
| | pp′-DDT |
| | ethyl-DDD |
| | HCH |
| | gamma-HCH |
| | lindane |
| | methoxychlor |
| | pentachlorophenol |
| | TDE |
| cyclodiene insecticides | aldrin |
| | bromocyclen |
| | chlorbicyclen |
| | chlordane |
| | chlordecone |
| | dieldrin |
| | dilor |
| | endosulfan |
| | alpha-endosulfan |
| | endrin |
| | HEOD |
| | heptachlor |
| | HHDN |
| | isobenzan |
| | isodrin |
| | kelevan |
| | mirex |
| organophosphorus insecticides | |
| organophosphate insecticides | bromfenvinfos |
| | calvinphos |
| | chlorfenvinphos |
| | crotoxyphos |
| | dichlorvos |
| | dicrotophos |
| | dimethylvinphos |
| | fospirate |
| | heptenophos |
| | methocrotophos |
| | mevinphos |
| | monocrotophos |
| | naled |
| | naftalofos |
| | phosphamidon |
| | propaphos |
| | TEPP |
| | tetrachlorvinphos |
| organothiophosphate insecticides | dioxabenzofos |
| | fosmethilan |
| | phenthoate |
| aliphatic organothiophosphate | acethion |
| insecticides | acetophos |
| | amiton |
| | cadusafos |
| | chlorethoxyfos |
| | chlormephos |
| | demephion |
| | demephion-O |
| | demephion-S |
| | demeton |
| | demeton-O |
| | demeton-S |
| | demeton-methyl |
| | demeton-O-methyl |
| | demeton-S-methyl |
| | demeton-S-methylsulphon |
| | disulfoton |
| | ethion |
| | ethoprophos |
| | IPSP |
| | isothioate |
| | malathion |
| | methacrifos |
| | methylacetophos |
| | oxydemeton-methyl |
| | oxydeprofos |
| | oxydisulfoton |
| | phorate |
| | sulfotep |
| | terbufos |
| | thiometon |
| aliphatic amide | amidithion |
| organothiophosphate insecticides | cyanthoate |
| | dimethoate |
| | ethoate-methyl |
| | formothion |
| | mecarbam |
| | omethoate |
| | prothoate |
| | sophamide |
| | vamidothion |
| oxime organothiophosphate | chlorphoxim |
| insecticides | phoxim |
| | phoxim-methyl |
| heterocyclic organothiophosphate | azamethiphos |
| insecticides | colophonate |
| | coumaphos |
| | coumithoate |
| | dioxathion |
| | endothion |
| | menazon |
| | morphothion |
| | phosalone |
| | pyraclofos |
| | pyrazothion |
| | pyridaphenthion |
| | quinothion |
| benzothiopyran | dithicrofos |
| organothiophosphate insecticides | thicrofos |
| benzotriazine organothiophosphate | azinphos-ethyl |
| insecticides | azinphos-methyl |
| isoindole organothiophosphate | dialifos |
| insecticides | phosmet |
| isoxazole organothiophosphate | isoxathion |
| insecticides | zolaprofos |
| pyrazolopyrimidine | chlorprazophos |
| organothiophosphate insecticides | pyrazophos |
| pyridine organothiophosphate | chlorpyrifos |
| insecticides | chlorpyrifos-methyl |
| pyrimidine organothiophosphate | butathiofos |
| insecticides | diazinon |
| | etrimfos |
| | lirimfos |
| | pirimioxyphos |
| | pirimiphos-ethyl |
| | pirimiphos-methyl |
| | primidophos |
| | pyrimitate |
| | tebupirimfos |
| quinoxaline organothiophosphate | quinalphos |
| insecticides | quinalphos-methyl |
| thiadiazole organothiophosphate | athidathion |
| insecticides | lythidathion |
| | methidathion |
| | prothidathion |
| triazole organothiophosphate | isazofos |
| insecticides | triazophos |
| phenyl organothiophosphate | azothoate |
| insecticides | bromophos |
| | bromophos-ethyl |
| | carbophenothion |
| | chlorthiophos |
| | cyanophos |
| | cythioate |
| | dicapthon |
| | dichlofenthion |
| | etaphos |
| | famphur |
| | fenchlorphos |
| | fenitrothion |
| | fensulfothion |
| | fenthion |
| | fenthion-ethyl |
| | heterophos |
| | jodfenphos |
| | mesulfenfos |
| | parathion |
| | parathion-methyl |
| | phenkapton |
| | phosnichlor |
| | profenofos |
| | prothiofos |
| | sulprofos |
| | temephos |
| | trichlormetaphos-3 |
| | trifenofos |
| | xiaochongliulin |
| phosphonate insecticides | butonate |
| | trichlorfon |
| phosphonothioate insecticides | mecarphon |
| phenyl ethylphosphonothioate | fonofos |
| insecticides | trichloronat |
| phenyl phenylphosphonothioate | cyanofenphos |
| insecticides | EPN |
| | leptophos |
| phosphoramidate insecticides | crufomate |
| | fenamiphos |
| | fosthietan |
| | mephosfolan |
| | phosfolan |
| | phosfolan-methyl |
| | pirimetaphos |
| phosphoramidothioate insecticides | acephate |
| | chloramine phosphorus |
| | isocarbophos |
| | isofenphos |
| | isofenphos-methyl |
| | methamidophos |
| | phosglycin |
| | propetamphos |
| phosphorodiamide insecticides | dimefox |
| | mazidox |
| | mipafox |
| | schradan |
| oxadiazine insecticides | indoxacarb |
| oxadiazolone insecticides | metoxadiazone |
| phthalimide insecticides | dialifos |
| | phosmet |
| | tetramethrin |
| physical insecticides | maltodextrin |
| desiccant insecticides | boric acid |
| | diatomaceous earth |
| | silica gel |
| pyrazole insecticides | chlorantraniliprole |
| | cyantraniliprole |
| | cyclaniliprole |
| | dimetilan |
| | isolan |
| | tebufenpyrad |
| | tetraniliprole |
| | tolfenpyrad |
| phenylpyrazole insecticides | acetoprole |
| | ethiprole |
| | fipronil |
| | flufiprole |
| | pyraclofos |
| | pyrafluprole |
| | pyriprole |
| | pyrolan |
| | vaniliprole |
| pyrethroid insecticides | |
| pyrethroid ester insecticides | acrinathrin |
| | allethrin |
| | bioallethrin |
| | esdépalléthrine |
| | barthrin |
| | bifenthrin |
| | kappa-bifenthrin |
| | bioethanomethrin |
| | brofenvalerate |
| | brofluthrinate |
| | bromethrin |
| | butethrin |
| | chlorempenthrin |
| | cyclethrin |
| | cycloprothrin |
| | cyfluthrin |
| | beta-cyfluthrin |
| | cyhalothrin |
| | gamma-cyhalothrin |
| | lambda-cyhalothrin |
| | cypermethrin |
| | alpha-cypermethrin |
| | beta-cypermethrin |
| | theta-cypermethrin |
| | zeta-cypermethrin |
| | cyphenothrin |
| | deltamethrin |
| | dimefluthrin |
| | dimethrin |
| | empenthrin |
| | d-fanshiluquebingjuzhi |
| | chloroprallethrin |
| | fenfluthrin |
| | fenpirithrin |
| | fenpropathrin |
| | fenvalerate |
| | esfenvalerate |
| | flucythrinate |
| | fluvalinate |
| | tau-fluvalinate |
| | furamethrin |
| | furethrin |
| | heptafluthrin |
| | imiprothrin |
| | japothrins |
| | kadethrin |
| | methothrin |
| | metofluthrin |
| | epsilon-metofluthrin |
| | momfluorothrin |
| | epsilon-momfluorothrin |
| | pentmethrin |
| | permethrin |
| | biopermethrin |
| | transpermethrin |
| | phenothrin |
| | prallethrin |
| | profluthrin |
| | proparthrin |
| | pyresmethrin |
| | renofluthrin |
| | meperfluthrin |
| | resmethrin |
| | bioresmethrin |
| | cismethrin |
| | tefluthrin |
| | kappa-tefluthrin |
| | terallethrin |
| | tetramethrin |
| | tetramethylfluthrin |
| | tralocythrin |
| | tralomethrin |
| | transfluthrin |
| | valerate |
| pyrethroid ether insecticides | etofenprox |
| | flufenprox |
| | halfenprox |
| | protrifenbute |
| | silafluofen |
| pyrethroid oxime insecticides | sulfoxime |
| | thiofluoximate |
| pyrimidinamine insecticides | flufenerim |
| | pyrimidifen |
| pyrrole insecticides | chlorfenapyr |
| quaternary ammonium insecticides | sanguinarine |
| sulfoximine insecticides | sulfoxaflor |
| tetramic acid insecticides | spirotetramat |
| tetronic acid insecticides | spiromesifen |
| thiazole insecticides | clothianidin |
| | imidaclothiz |
| | thiamethoxam |
| | thiapronil |
| thiazolidine insecticides | tazimcarb |
| | thiacloprid |
| thiourea insecticides | diafenthiuron |
| urea insecticides | flucofuron |
| | sulcofuron |
| zwitterionic insecticides | dicloromezotiaz |
| | triflumezopyrim |
| unclassified insecticides | afidopyropen |
| | afoxolaner |
| | allosamidin |
| | closantel |
| | copper naphthenate |
| | crotamiton |
| | EXD |
| | fenazaflor |
| | fenoxacrim |
| | flometoquin |
| | flonicamid |
| | fluhexafon |
| | flupyradifurone |
| | fluralaner |
| | fluxametamide |
| | hydramethylnon |
| | isoprothiolane |
| | jiahuangchongzong |
| | malonoben |
| | metaflumizone |
| | nifluridide |
| | plifenate |
| | pyridaben |
| | pyridalyl |
| | pyrifluquinazon |
| | rafoxanide |
| | thuringiensin |
| | triarathene |
| | triazamate |
| ACARICIDES | |
| botanical acaricides | carvacrol |
| | sanguinarine |
| bridged diphenyl acaricides | azobenzene |
| | benzoximate |
| | benzyl benzoate |
| | bromopropylate |
| | chlorbenside |
| | chlorfenethol |
| | chlorfenson |
| | chlorfensulphide |
| | chlorobenzilate |
| | chloropropylate |
| | cyflumetofen |
| | DDT |
| | dicofol |
| | diphenyl sulfone |
| | dofenapyn |
| | fenson |
| | fentrifanil |
| | fluorbenside |
| | genit |
| | hexachlorophene |
| | phenproxide |
| | proclonol |
| | tetradifon |
| | tetrasul |
| carbamate acaricides | benomyl |
| | carbanolate |
| | carbaryl |
| | carbofuran |
| | methiocarb |
| | metolcarb |
| | promacyl |
| | propoxur |
| oxime carbamate acaricides | aldicarb |
| | butocarboxim |
| | oxamyl |
| | thiocarboxime |
| | thiofanox |
| carbazate acaricides | bifenazate |
| dinitrophenol acaricides | binapacryl |
| | dinex |
| | dinobuton |
| | dinocap |
| | dinocap-4 |
| | dinocap-6 |
| | dinocton |
| | dinopenton |
| | dinosulfon |
| | dinoterbon |
| | DNOC |
| formamidine acaricides | amitraz |
| | chlordimeform |
| | chloromebuform |
| | formetanate |
| | formparanate |
| | medimeform |
| | semiamitraz |
| macrocyclic lactone acaricides | tetranactin |
| avermectin acaricides | abamectin |
| | doramectin |
| | eprinomectin |
| | ivermectin |
| | selamectin |
| milbemycin acaricides | milbemectin |
| | milbemycinoxime |
| | moxidectin |
| mite growth regulators | clofentezine |
| | cyromazine |
| | diflovidazin |
| | dofenapyn |
| | fluazuron |
| | flubenzimine |
| | flucycloxuron |
| | flufenoxuron |
| | hexythiazox |
| organochlorine acaricides | bromocyclen |
| | camphechlor |
| | DDT |
| | dienochlor |
| | endosulfan |
| | lindane |
| organophosphorus acaricides | |
| organophosphate acaricides | chlorfenvinphos |
| | crotoxyphos |
| | dichlorvos |
| | heptenophos |
| | mevinphos |
| | monocrotophos |
| | naled |
| | TEPP |
| | tetrachlorvinphos |
| organothiophosphate acaricides | amidithion |
| | amiton |
| | azinphos-ethyl |
| | azinphos-methyl |
| | azothoate |
| | benoxafos |
| | bromophos |
| | bromophos-ethyl |
| | carbophenothion |
| | chlorpyrifos |
| | chlorthiophos |
| | coumaphos |
| | cyanthoate |
| | demeton |
| | demeton-O |
| | demeton-S |
| | demeton-methyl |
| | demeton-O-methyl |
| | demeton-S-methyl |
| | demeton-S-methylsulphon |
| | dialifos |
| | diazinon |
| | dimethoate |
| | dioxathion |
| | disulfoton |
| | endothion |
| | ethion |
| | ethoate-methyl |
| | formothion |
| | malathion |
| | mecarbam |
| | methacrifos |
| | omethoate |
| | oxydeprofos |
| | oxydisulfoton |
| | parathion |
| | phenkapton |
| | phorate |
| | phosalone |
| | phosmet |
| | phostin |
| | phoxim |
| | pirimiphos-methyl |
| | prothidathion |
| | prothoate |
| | pyrimitate |
| | quinalphos |
| | quintiofos |
| | sophamide |
| | sulfotep |
| | thiometon |
| | triazophos |
| | trifenofos |
| | vamidothion |
| phosphonate acaricides | trichlorfon |
| phosphoramidothioate acaricides | isocarbophos |
| | methamidophos |
| | propetamphos |
| phosphorodiamide acaricides | dimefox |
| | mipafox |
| | schradan |
| organotin acaricides | azocyclotin |
| | cyhexatin |
| | fenbutatin oxide |
| | phostin |
| phenylsulfamide acaricides | dichlofluanid |
| phthalimide acaricides | dialifos |
| | phosmet |
| pyrazole acaricides | cyenopyrafen |
| | fenpyroximate |
| | pyflubumide |
| | tebufenpyrad |
| phenylpyrazole acaricides | acetoprole |
| | fipronil |
| | vaniliprole |
| pyrethroid acaricides | |
| pyrethroid ester acaricides | acrinathrin |
| | bifenthrin |
| | brofluthrinate |
| | cyhalothrin |
| | cypermethrin |
| | alpha-cypermethrin |
| | fenpropathrin |
| | fenvalerate |
| | flucythrinate |
| | flumethrin |
| | fluvalinate |
| | tau-fluvalinate |
| | permethrin |
| pyrethroid ether acaricides | halfenprox |
| pyrimidinamine acaricides | pyrimidifen |
| pyrrole acaricides | chlorfenapyr |
| quaternary ammonium acaricides | sanguinarine |
| quinoxaline acaricides | chinomethionat |
| | thioquinox |
| strobilurin acaricides | |
| methoxyacrylate strobilurin acaricides | bifujunzhi |
| | fluacrypyrim |
| | flufenoxystrobin |
| | pyriminostrobin |
| sulfite ester acaricides | aramite |
| | propargite |
| tetronic acid acaricides | spirodiclofen |
| tetrazine acaricides | clofentezine |
| | diflovidazin |
| thiazolidine acaricides | flubenzimine |
| | hexythiazox |
| thiocarbamate acaricides | fenothiocarb |
| thiourea acaricides | chloromethiuron |
| | diafenthiuron |
| unclassified acaricides | acequinocyl |
| | afoxolaner |
| | amidoflumet |
| | arsenous oxide |
| | clenpirin |
| | closantel |
| | crotamiton |
| | cycloprate |
| | cymiazole |
| | disulfiram |
| | etoxazole |
| | fenazaflor |
| | fenazaquin |
| | fluenetil |
| | fluralaner |
| | mesulfen |
| | MNAF |
| | nifluridide |
| | nikkomycins |
| | pyridaben |
| | sulfiram |
| | sulfluramid |
| | sulfur |
| | thuringiensin |
| | triarathene |
| CHEMOSTERILANTS | |
| | apholate |
| | bisazir |
| | busulfan |
| | diflubenzuron |
| | dimatif |
| | hemel |
| | hempa |
| | metepa |
| | methiotepa |
| | methylapholate |
| | morzid |
| | penfluron |
| | tepa |
| | thiohempa |
| | thiotepa |
| | tretamine |
| | uredepa |
| INSECT REPELLENTS | |
| | acrep |
| | butopyronoxyl |
| | camphor |
| | d-camphor |
| | carboxide |
| | dibutyl phthalate |
| | diethyltoluamide |
| | dimethyl carbate |
| | dimethyl phthalate |
| | dibutyl succinate |
| | ethohexadiol |
| | hexamide |
| | icaridin |
| | methoquin-butyl |
| | methylneodecanamide |
| | 2-(octylthio)ethanol |
| | oxamate |
| | quwenzhi |
| | quyingding |
| | rebemide |
| | zengxiaoan |
| NEMATICIDES | |
| avermectin nematicides | abamectin |
| botanical nematicides | carvacrol |
| carbamate nematicides | benomyl |
| | carbofuran |
| | carbosulfan |
| | cloethocarb |
| oxime carbamate nematicides | alanycarb |
| | aldicarb |
| | aldoxycarb |
| | oxamyl |
| | tirpate |
| fumigant nematicides | carbon disulfide |
| | cyanogen |
| | 1,2-dichloropropane |
| | 1,3-dichloropropene |
| | dithioether |
| | methyl bromide |
| | methyl iodide |
| | sodium tetrathiocarbonate |
| organophosphorus nematicides | |
| organophosphate nematicides | diamidafos |
| | fenamiphos |
| | fosthietan |
| | phosphamidon |
| organothiophosphate nematicides | cadusafos |
| | chlorpyrifos |
| | dichlofenthion |
| | dimethoate |
| | ethoprophos |
| | fensulfothion |
| | fosthiazate |
| | heterophos |
| | isamidofos |
| | isazofos |
| | phorate |
| | phosphocarb |
| | terbufos |
| | thionazin |
| | triazophos |
| phosphonothioate nematicides | imicyafos |
| | mecarphon |
| unclassified nematicides | acetoprole |
| | benclothiaz |
| | chloropicrin |
| | dazomet |
| | DBCP |
| | DCIP |
| | fluazaindolizine |
| | fluensulfone |
| | furfural |
| | metam |
| | methyl isothiocyanate |
| | tioxazafen |
| | xylenols |

From www.alanwood.net

Insecticides also include synergists or activators that are not in themselves considered toxic or insecticidal, but are materials used with insecticides to synergize or enhance the activity of the insecticides. Syngergists or activators include piperonyl butoxide. Insecticides can be biorational, or can also be known as biopesticides or biological pesticides. Biorational refers to any substance of natural origin (or man-made substances resembling those of natural origin) that has a detrimental or lethal effect on specific target pest(s), e.g., insects, weeds, plant diseases (including nematodes), and vertebrate pests, possess a unique mode of action, are non-toxic to man, domestic plants and animals, and have little or no adverse effects on wildlife and the environment. Biorational insecticides (or biopesticides or biological pesticides) can be grouped as: (1) biochemicals (hormones, enzymes, pheromones and natural agents, such as insect and plant growth regulators), (2) microbial (viruses, bacteria, fungi, protozoa, and nematodes), or (3) Plant-Incorporated protectants (PIPs)—primarily transgenic plants, e.g., Bt corn.

As used herein, the term “locus” (plural: “loci”) refers to any site that has been defined genetically. A locus may be a gene, or part of a gene, or a DNA sequence that has some regulatory role, and may be occupied by different sequences.

As used herein, the term “allele” or “alleles” means any of one or more alternative forms of a gene, all of which alleles relate to at least one trait or characteristic. In a diploid cell, the two alleles of a given gene occupy corresponding loci on a pair of homologous chromosomes. Alleles are considered identical when they express a similar phenotype. For example, an “R” allele can be a form of a given gene in a pest that confers resistance to an insecticidal trait or chemical insecticide. An “S” allele can be a form of the same given gene in a pest that confers susceptibility to an insecticidal trait or chemical insecticide.

As used herein, the term “heterozygote” refers to a diploid or polyploid individual cell, plant or pest having different alleles (forms of a given gene) present at least at one locus.

As used herein, the term “heterozygous” refers to the presence of different alleles (forms of a given gene) at a particular gene locus. For example, a pest heterozygous for resistance to an insecticidal trait or chemical insecticide can be “RS” or “SR”, that is, comprising both a resistant “R” allele and a susceptible “S” allele.

As used herein, the term “homozygote” refers to an individual cell, plant or pest having the same alleles at one or more loci.

As used herein, the term “homozygous” refers to the presence of identical alleles at one or more loci in homologous chromosomal segments. For example, a pest homozygous for resistance to an insecticidal trait or chemical insecticide comprises “RR” alleles, while a pest homozygous for susceptibility to an insecticidal trait or chemical insecticide comprises “SS” alleles.

As used herein, the term “high-dose” refers to an insecticide (chemical or transgenic) concentration that is sufficiently high such that the resistance allele is rendered recessive. That is, only the homozygote RR members of the population are resistant.

As used herein, the term “low-dose” refers to an insecticide (chemical or transgenic) concentration that is reasonably low such that the resistance allele is rendered dominant. That is, both RS and SR heterozygotes are resistant.

As used herein, the term “fitness” refers to a property of the individual and comprises the ability of an individual to survive and reproduce in a given environment.

As used herein, the phrase “fitness differential under selection pressure by the insecticide” refers to the fitness advantage of resistant phenotypes over susceptible phenotypes when both are exposed to the insecticide (Andow 2008).

As used herein, the phrase “fitness cost of resistance (in the absence of the insecticide)” refers to the fitness advantage of susceptible phenotypes over resistant phenotypes in the absence of the insecticide.

As used herein, “Integrated Pest Management” or “IPM” refers to a comprehensive approach to pest control that uses combined means to reduce the status of pests to tolerable levels while maintaining a quality environment.

As used herein, “mode of action” or “MoA” refers to the basis for which a given insecticide or acaricide operates to injure or kill a pest. Compounds within a specific chemical group usually share a common target site within the pest, and thus share a common Mode of Action. Orthogonal MoAs share little or no overlap in target sites.

As used herein, “kairomone” refers to a compound that is an interspecific chemical message that benefits the receiving species and disadvantages the emitting species. In one embodiment, kairomones can act between two insect species for location of host insects by parasitoids. In another embodiment, kairomones can act between an insect and a plant for location of host plants by herbivores or for location of herbivore-damaged plants by parasitoids.

As used herein, “mating disruption” refers to a pest management technique or tactic that involves the use of sex pheromones to disrupt the reproductive cycle of insects. For example, mating disruption exploits the male cotton bollworm's natural response to follow the pheromone plume by introducing pheromone unconnected to a female cotton bollworm into the insects' habitat. The general effect of mating disruption may possibly be to impair the male cotton bollworm's normal semiochemically-mediated behavior by masking the natural pheromone plumes, causing the males to follow “false pheromone trails” at the expense of finding mates, and affecting the males' ability to respond to “calling” females. Mating disruption may alternatively raise the response threshold or saturate the male's senses with the high pheromone concentration, so that the male can no longer sense the small amount of pheromone released by the female. Consequently, the male population experiences a reduced probability of successfully locating and mating with female cotton bollworms.

As used herein, “pest” or “pests” refer to organisms possessing characteristics that are considered damaging or unwanted. Pests can include insects, animals, plants, molds, fungi, bacteria and viruses. For example, the Grape Berry Moth (GBM) (Endopiza viteana Clemens) is one of the principal insect pests of grape. As another example, the primary pest of cherry is a fruit fly, but several Lepidoptera, including oblique banded leafroller (OBLR) (Choristoneura rosacean Harris), can cause significant crop loss as well. Other Lepidoptera pests include moths and butterflies of Cossidae, Psychidae, Noctuidae, Pieridae, Lymantriidae, Geometridae, Anthelidae, Saturniidae, Thyrididae, Limacodidae, Pyralidae and Hyblaeidae families. As a further example, moths such as the cotton bollworm and the corn earworm in the Noctuidae family (Helicoverpa armigera and Helicoverpa zea) are major pests for crops such as corn, tomatoes and soybean. As another example, mites such as Tetranychus urticae attack a wide range of plants including peppers, tomatoes, potatoes, beans, corn, cannabis and strawberries. As a further example, the navel orangeworm (Amyelois transitella) is a moth of the Pyralidae family native to the southwestern United States and Mexico and is a commercial pest to a number of crops including walnut trees (Juglans regia), common fig (Ficus carica), almond trees (Prunus dulcis), and pistachio trees (Pistacia vera). As another example, the citrus leafminer (Phyllocnistis citrella), or CLM, is a moth of the Gracillariidae family found all over the world. The CLM larvae infest citrus species such as bael tree (Aegle marmelos), Atalantia tree species, calamondin (Citrofortunella microcarpa), lemon tree (Citrus limon), grapefruit (Citrus paradisi), pomelo (Citrus maxima), kumquat (Fortunella margarita), Murraya paniculata ornamental tree or hedge, and trifoliate orange (Poncirus trifoliate), by mining their leaves, creating epidermal corridors with well-marked central frass lines. Effective control of these and other pests is a primary goal of agriculture.

As used herein, “pest control” refers to inhibition of pest development (including mortality, feeding reduction, and/or mating disruption).

As used herein, “pesticide” refers to a compound or substance that repels, incapacitates or kills a pest, such as an insect, weed or pathogen. Thus pesticides can encompass, but are not limited to, acaricides, algicides, antifeedants, avicides, bactericides, bird repellents, chemosterilants, fungicides, herbicide safeners, herbicides, insect repellents, insecticides, mammal repellents, mating disrupters, molluscicides, nematicides, plant activators, plant growth regulators, rodenticides, synergists and virucides.

As used herein, “acaricide” refers to pesticides that kill members of the arachnid subclass Acari, which includes ticks and mites.

As used herein, “arachnid” refers to a class of joint-legged invertebrate animals, also known as arthropods, in the subphylum Chelicerata. Arachnids have eight legs as opposed to the six legs found on insects. Also in contrast to insects, arachnids do not have antennae or wings. Arachnids also have two further pairs of appendages that are adapted for feeding, defense, and sensory perception. The first pair, the chelicerae, serves in feeding and defense. The second pair of appendages, the pedipalps, has been adapted for feeding, locomotion, and/or reproductive functions. The body is organized into the cephalothorax, a fusion of the head and thorax, and the abdomen. There are over 100,000 species of arachnids and include spiders, scorpions, harvestmen, ticks, mites and solifuges.

As used herein, “mite” refers to a small arthropod belonging to the subclass Acari (or Acarina) and the class Arachnida. About 48, 200 species of mites have been described. Mites actively engage in the fragmentation and mixing of organic matter in soil ecosystems. Mites occur in many habitats and eat a wide variety of material including living and dead plant and fungal matter, lichens and carrion. Many mites are parasitic on plants and animals. For example, mites of the family Pyroglyphidae, or nest mites, live primarily in the nests of birds and animals and consume blood, skin and keratin. Dust mites, which feed on dead skin and hair shed from humans, evolved from these parasitic ancestors. Examples of parasitic mites that infest insects include Varroa destructor, which attaches to the body of the honey bee, and Acarapis woodi (family Tarsonemidae), which lives in the tracheae of honey bees. Mites that are considered plant pests include spider mites (family Tetranychidae), thread-footed mites (family Tarsonemidae), and the gall mites (family Eriophyidae). Among the species that attack animals are members of the sarcoptic mange mites (family Sarcoptidae), which burrow under the skin. Demodex mites (family Demodicidae) are parasites that live in or near the hair follicles of mammals, including humans.

As used herein, the term “phagostimulant” refers to one or more compounds, substances or compositions that can be tasted by an organism, such as an insect pest, and that generally stimulates feeding. In one embodiment, a phagostimulant can be found in one or more plants. In another embodiment, a phagostimulant can be synthesized or produced in vitro. In another embodiment, a phagostimulant can be formulated for one or more broadcast sprays. In yet another embodiment, a phagostimulant can be formulated for one or more feeding stations. In one embodiment, a phagostimulant can comprise carbohydrates, proteins, amino acids and/or various lipids. In another embodiment, a phagostimulant can comprise one or more essential nutrients. In yet another embodiment, a phagostimulant can signal to an organism that the organism is feeding on the right food. In a further embodiment, a phagostimulant can be a deterrent.

As used herein, the terms “pheromone” or “natural pheromone,” when used in reference to an insect pheromone, is intended to mean the volatile chemical or particular volatile chemical blend having a chemical structure corresponding to the chemical structure of a pheromone that is released by a particular insect for the function of chemical communication within the species. For example, a female moth releases pheromones, which are detected by sensors on the antennae of a male moth and enable the male moth to locate the female moth for mating. As another example, the pheromone blend for Spodoptera frugiperda comprises (Z)-9-tetradecenyl acetate (Z9-14Ac):(Z)-11-hexadecenyl acetate (Z11-16Ac) or (Z)-9-tetradecenyl acetate (Z9-14Ac):(Z)-11-hexadecenyl acetate (Z11-16Ac). (Z)-7-dodecenyl acetate (Z7-12Ac). In one embodiment, the ratio of (Z)-9-tetradecenyl acetate (Z9-14Ac):(Z)-11-hexadecenyl acetate (Z11-16Ac) pheromone blend is about 87:13. In another embodiment, the ratio of (Z)-9-tetradecenyl acetate (Z9-14Ac):(Z)-11-hexadecenyl acetate (Z11-16Ac):(Z)-7-dodecenyl acetate (Z7-12Ac) pheromone blend is about 87:12:1. As a further example, the pheromone blend for Helicoverpa zea comprises (Z)-11-hexadecenal (Z11-16Ald):(Z)-9-hexadecenal (Z9-16:Ald). In one embodiment, the ratio of (Z)-11-hexadecenal (Z11-16Ald):(Z)-9-hexadecenal (Z9-16:Ald) pheromone blend is about 97:3. As used herein, the term “non-natural” or “non-naturally occurring,” when used in reference to a synthetic pheromone, is intended to mean a molecule that is not produced by the particular insect species whose behavior is modified using said molecule. A list of representative pheromones is given in Table 2.

TABLE 2
Representative pheromones

| Name | Name |
|---|---|
| (E)-2-Decen-1-ol | (E)-5-Dodecenyl acetate |
| (E)-2-Decenyl acetate | (Z)-5-Dodecen-1-ol |
| (E)-2-Decenal | (Z)-5-Dodecenyl acetate |
| (Z)-2-Decen-1-ol | (Z)-5-Dodecenal |
| (Z)-2-Decenyl acetate | (E)-6-Dodecen-1-ol |
| (Z)-2-Decenal | (Z)-6-Dodecenyl acetate |
| (E)-3-Decen-1-ol | (E)-6-Dodecenal |
| (Z)-3-Decenyl acetate | (E)-7-Dodecen-1-ol |
| (Z)-3-Decen-1-ol | (E)-7-Dodecenyl acetate |
| (Z)-4-Decen-1-ol | (E)-7-Dodecenal |
| (E)-4-Decenyl acetate | (Z)-7-Dodecen-1-ol |
| (Z)-4-Decenyl acetate | (Z)-7-Dodecenyl acetate |
| (Z)-4-Decenal | (Z)-7-Dodecenal |
| (E)-5-Decen-1-ol | (E)-8-Dodecen-1-ol |
| (E)-5-Decenyl acetate | (E)-8-Dodecenyl acetate |
| (Z)-5-Decen-1-ol | (E)-8-Dodecenal |
| (Z)-5-Decenyl acetate | (Z)-8-Dodecen-1-ol |
| (Z)-5-Decenal | (Z)-8-Dodecenyl acetate |
| (E)-7-Decenyl acetate | (E)-9-Dodecen-1-ol |
| (Z)-7-Decenyl acetate | (E)-9-Dodecenyl acetate |
| (E)-8-Decen-1-ol | (E)-9-Dodecenal |
| (E,E)-2,4-Decadienal | (Z)-9-Dodecen-1-ol |
| (E,Z)-2,4-Decadienal | (Z)-9-Dodecenyl acetate |
| (Z,Z)-2,4-Decadienal | (Z)-9-Dodecenal |
| (E,E)-3,5-Decadienyl acetate | (E)-10-Dodecen-1-ol |
| (Z,E)-3,5-Decadienyl acetate | (E)-10-Dodecenyl acetate |
| (Z,Z)-4,7-Decadien-1-ol | (E)-10-Dodecenal |
| (Z,Z)-4,7-Decadienyl acetate | (Z)-10-Dodecen-1-ol |
| (E)-2-Undecenyl acetate | (Z)-10-Dodecenyl acetate |
| (E)-2-Undecenal | (E,Z)-3,5-Dodecadienyl acetate |
| (Z)-5-Undecenyl acetate | (Z,E)-3,5-Dodecadienyl acetate |
| (Z)-7-Undecenyl acetate | (Z,Z)-3,6-Dodecadien-1-ol |
| (Z)-8-Undecenyl acetate | (E,E)-4,10-Dodecadienyl acetate |
| (Z)-9-Undecenyl acetate | (E,E)-5,7-Dodecadien-1-ol |
| (E)-2-Dodecenal | (E,E)-5,7-Dodecadienyl acetate |
| (Z)-3-Dodecen-1-ol | (E,Z)-5,7-Dodecadien-1-ol |
| (E)-3-Dodecenyl acetate | (E,Z)-5,7-Dodecadienyl acetate |
| (Z)-3-Dodecenyl acetate | (E,Z)-5,7-Dodecadienal |
| (E)-4-Dodecenyl acetate | (Z,E)-5,7-Dodecadien-1-ol |
| (E)-5-Dodecen-1-ol | (Z,E)-5,7-Dodecadienyl acetate |
| (Z,E)-5,7-Dodecadienal | (Z,Z)-4,7-Tridecadien-1-ol |
| (Z,Z)-5,7-Dodecadienyl acetate | (Z,Z)-4,7-Tridecadienyl acetate |
| (Z,Z)-5,7-Dodecadienal | (E,Z)-5,9-Tridecadienyl acetate |
| (E,E)-7,9-Dodecadienyl acetate | (Z,E)-5,9-Tridecadienyl acetate |
| (E,Z)-7,9-Dodecadien-1-ol | (Z,Z)-5,9-Tridecadienyl acetate |
| (E,Z)-7,9-Dodecadienyl acetate | (Z,Z)-7,11-Tridecadienyl acetate |
| (E,Z)-7,9-Dodecadienal | (E,Z,Z)-4,7,10-Tridecatrienyl acetate |
| (Z,E)-7,9-Dodecadien-1-ol | (E)-3-Tetradecen-1-ol |
| (Z,E)-7,9-Dodecadienyl acetate | (E)-3-Tetradecenyl acetate |
| (Z,Z)-7,9-Dodecadien-1-ol | (Z)-3-Tetradecen-1-ol |
| (Z,Z)-7,9-Dodecadienyl acetate | (Z)-3-Tetradecenyl acetate |
| (E,E)-8,10-Dodecadien-1-ol | (E)-5-Tetradecen-1-ol |
| (E,E)-8,10-Dodecadienyl acetate | (E)-5-Tetradecenyl acetate |
| (E,E)-8,10-Dodecadienal | (E)-5-Tetradecenal |
| (E,Z)-8,10-Dodecadien-1-ol | (Z)-5-Tetradecen-1-ol |
| (E,Z)-8,10-Dodecadienyl acetate | (Z)-5-Tetradecenyl acetate |
| (E,Z)-8,10-Dodecadienal | (Z)-5-Tetradecenal |
| (Z,E)-8,10-Dodecadien-1-ol | (E)-6-Tetradecenyl acetate |
| (Z,E)-8,10-Dodecadienyl acetate | (Z)-6-Tetradecenyl acetate |
| (Z,E)-8,10-Dodecadienal | (E)-7-Tetradecen-1-ol |
| (Z,Z)-8,10-Dodecadien-1-ol | (E)-7-Tetradecenyl acetate |
| (Z,Z)-8,10-Dodecadienyl acetate | (Z)-7-Tetradecen-1-ol |
| (Z,E,E)-3,6,8-Dodecatrien-1-ol | (Z)-7-Tetradecenyl acetate |
| (Z,Z,E)-3,6,8-Dodecatrien-1-ol | (Z)-7-Tetradecenal |
| (E)-2-Tridecenyl acetate | (E)-8-Tetradecenyl acetate |
| (Z)-2-Tridecenyl acetate | (Z)-8-Tetradecen-1-ol |
| (E)-3-Tridecenyl acetate | (Z)-8-Tetradecenyl acetate |
| (E)-4-Tridecenyl acetate | (Z)-8-Tetradecenal |
| (Z)-4-Tridecenyl acetate | (E)-9-Tetradecen-1-ol |
| (Z)-4-Tridecenal | (E)-9-Tetradecenyl acetate |
| (E)-6-Tridecenyl acetate | (Z)-9-Tetradecen-1-ol |
| (Z)-7-Tridecenyl acetate | (Z)-9-Tetradecenyl acetate |
| (E)-8-Tridecenyl acetate | (Z)-9-Tetradecenal |
| (Z)-8-Tridecenyl acetate | (E)-10-Tetradecenyl acetate |
| (E)-9-Tridecenyl acetate | (Z)-10-Tetradecenyl acetate |
| (Z)-9-Tridecenyl acetate | (E)-11-Tetradecen-1-ol |
| (Z)-10-Tridecenyl acetate | (E)-11-Tetradecenyl acetate |
| (E)-11-Tridecenyl acetate | (E)-11-Tetradecenal |
| (Z)-11-Tridecenyl acetate | (Z)-11-Tetradecen-1-ol |
| (E,Z)-4,7-Tridecadienyl acetate | (Z)-11-Tetradecenyl acetate |
| (Z)-11-Tetradecenal | (E,E)-10,12-Tetradecadienal |
| (E)-12-Tetradecenyl acetate | (E,Z)-10,12-Tetradecadienyl acetate |
| (Z)-12-Tetradecenyl acetate | (Z,E)-10,12-Tetradecadienyl acetate |
| (E,E)-2,4-Tetradecadienal | (Z,Z)-10,12-Tetradecadien-1-ol |
| (E,E)-3,5-Tetradecadienyl acetate | (Z,Z)-10,12-Tetradecadienyl acetate |
| (E,Z)-3,5-Tetradecadienyl acetate | (E,Z,Z)-3,8,11-Tetradecatrienyl |
| | acetate |
| (Z,E)-3,5-Tetradecadienyl acetate | (E)-8-Pentadecen-1-ol |
| (E,Z)-3,7-Tetradecadienyl acetate | (E)-8-Pentadecenyl acetate |
| (E,Z)-3,8-Tetradecadienyl acetate | (Z)-8-Pentadecen-1-ol |
| (E,Z)-4,9-Tetradecadienyl acetate | (Z)-8-Pentadecenyl acetate |
| (E,Z)-4,9-Tetradecadienal | (Z)-9-Pentadecenyl acetate |
| (E,Z)-4,10-Tetradecadienyl acetate | (E)-9-Pentadecenyl acetate |
| (E,E)-5,8-Tetradecadienal | (Z)-10-Pentadecenyl acetate |
| (Z,Z)-5,8-Tetradecadien-1-ol | (Z)-10-Pentadecenal |
| (Z,Z)-5,8-Tetradecadienyl acetate | (E)-12-Pentadecenyl acetate |
| (Z,Z)-5,8-Tetradecadienal | (Z)-12-Pentadecenyl acetate |
| (E,E)-8,10-Tetradecadien-1-ol | (Z,Z)-6,9-Pentadecadien-1-ol |
| (E,E)-8,10-Tetradecadienyl acetate | (Z,Z)-6,9-Pentadecadienyl acetate |
| (E,E)-8,10-Tetradecadienal | (Z,Z)-6,9-Pentadecadienal |
| (E,Z)-8,10-Tetradecadienyl acetate | (E,E)-8,10-Pentadecadienyl acetate |
| (E,Z)-8,10-Tetradecadienal | (E,Z)-8,10-Pentadecadien-1-ol |
| (Z,E)-8,10-Tetradecadien-1-ol | (E,Z)-8,10-Pentadecadienyl acetate |
| (Z,E)-8,10-Tetradecadienyl acetate | (Z,E)-8,10-Pentadecadienyl acetate |
| (Z,Z)-8,10-Tetradecadienal | (Z,Z)-8,10-Pentadecadienyl acetate |
| (E,E)-9,11-Tetradecadienyl acetate | (E,Z)-9,11-Pentadecadienal |
| (E,Z)-9,11-Tetradecadienyl acetate | (Z,Z)-9,11-Pentadecadienal |
| (Z,E)-9,11-Tetradecadien-1-ol | (Z)-3-Hexadecenyl acetate |
| (Z,E)-9,11-Tetradecadienyl acetate | (E)-5-Hexadecen-1-ol |
| (Z,E)-9,11-Tetradecadienal | (E)-5-Hexadecenyl acetate |
| (Z,Z)-9,11-Tetradecadien-1-ol | (Z)-5-Hexadecen-1-ol |
| (Z,Z)-9,11-Tetradecadienyl acetate | (Z)-5-Hexadecenyl acetate |
| (Z,Z)-9,11-Tetradecadienal | (E)-6-Hexadecenyl acetate |
| (E,E)-9,12-Tetradecadienyl acetate | (E)-7-Hexadecen-1-ol |
| (Z,E)-9,12-Tetradecadien-1-ol | (E)-7-Hexadecenyl acetate |
| (Z,E)-9,12-Tetradecadienyl acetate | (E)-7-Hexadecenal |
| (Z,E)-9,12-Tetradecadienal | (Z)-7-Hexadecen-1-ol |
| (Z,Z)-9,12-Tetradecadien-1-ol | (Z)-7-Hexadecenyl acetate |
| (Z,Z)-9,12-Tetradecadienyl acetate | (Z)-7-Hexadecenal |
| (E,E)-10,12-Tetradecadien-1-ol | (E)-8-Hexadecenyl acetate |
| (E,E)-10,12-Tetradecadienyl | (E)-9-Hexadecen-1-ol |
| acetate | |
| (E)-9-Hexadecenyl acetate | (E,E)-10,12-Hexadecadien-1-ol |
| (E)-9-Hexadecenal | (E,E)-10,12-Hexadecadienyl acetate |
| (Z)-9-Hexadecen-1-ol | (E,E)-10,12-Hexadecadienal |
| (Z)-9-Hexadecenyl acetate | (E,Z)-10,12-Hexadecadien-1-ol |
| (Z)-9-Hexadecenal | (E,Z)-10,12-Hexadecadienyl acetate |
| (E)-10-Hexadecen-1-ol | (E,Z)-10,12-Hexadecadienal |
| (E)-10-Hexadecenal | (Z,E)-10,12-Hexadecadienyl acetate |
| (Z)-10-Hexadecenyl acetate | (Z,E)-10,12-Hexadecadienal |
| (Z)-10-Hexadecenal | (Z,Z)-10,12-Hexadecadienal |
| (E)-11-Hexadecen-1-ol | (E,E)-11,13-Hexadecadien-1-ol |
| (E)-11-Hexadecenyl acetate | (E,E)-11,13-Hexadecadienyl acetate |
| (E)-11-Hexadecenal | (E,E)-11,13-Hexadecadienal |
| (Z)-11-Hexadecen-1-ol | (E,Z)-11,13-Hexadecadien-1-ol |
| (Z)-11-Hexadecenyl acetate | (E,Z)-11,13-Hexadecadienyl acetate |
| (Z)-11-Hexadecenal | (E,Z)-11,13-Hexadecadienal |
| (Z)-12-Hexadecenyl acetate | (Z,E)-11,13-Hexadecadien-1-ol |
| (Z)-12-Hexadecenal | (Z,E)-11,13-Hexadecadienyl acetate |
| (E)-14-Hexadecenal | (Z,E)-11,13-Hexadecadienal |
| (Z)-14-Hexadecenyl acetate | (Z,Z)-11,13-Hexadecadien-1-ol |
| (E,E)-1,3-Hexadecadien-1-ol | (Z,Z)-11,13-Hexadecadienyl acetate |
| (E,Z)-4,6-Hexadecadien-1-ol | (Z,Z)-11,13-Hexadecadienal |
| (E,Z)-4,6-Hexadecadienyl acetate | (E,E)-10,14-Hexadecadienal |
| (E,Z)-4,6-Hexadecadienal | (Z,E)-11,14-Hexadecadienyl acetate |
| (E,Z)-6,11-Hexadecadienyl acetate | (E,E,Z)-4,6,10-Hexadecatrien-1-ol |
| (E,Z)-6,11-Hexadecadienal | (E,E,Z)-4,6,10-Hexadecatrienyl |
| | acetate |
| (Z,Z)-7,10-Hexadecadien-1-ol | (E,Z,Z)-4,6,10-Hexadecatrien-1-ol |
| (Z,Z)-7,10-Hexadecadienyl acetate | (E,Z,Z)-4,6,10-Hexadecatrienyl |
| | acetate |
| (Z,E)-7,11-Hexadecadien-1-ol | (E,E,Z)-4,6,11-Hexadecatrienyl |
| | acetate |
| (Z,E)-7,11-Hexadecadienyl acetate | (E,E,Z)-4,6,11-Hexadecatrienal |
| (Z,E)-7,11-Hexadecadienal | (Z,Z,E)-7,11,13-Hexadecatrienal |
| (Z,Z)-7,11-Hexadecadien-1-ol | (E,E,E)-10,12,14-Hexadecatrienyl |
| | acetate |
| (Z,Z)-7,11-Hexadecadienyl acetate | (E,E,E)-10,12,14-Hexadecatrienal |
| (Z,Z)-7,11-Hexadecadienal | (E,E,Z)-10,12,14-Hexadecatrienyl |
| | acetate |
| (Z,Z)-8,10-Hexadecadienyl acetate | (E,E,Z)-10,12,14-Hexadecatrienal |
| (E,Z)-8,11-Hexadecadienal | (E,E,Z,Z)-4,6,11,13- |
| | Hexadecatetraenal |
| (E,E)-9,11-Hexadecadienal | (E)-2-Heptadecenal |
| (E,Z)-9,11-Hexadecadienyl acetate | (Z)-2-Heptadecenal |
| (E,Z)-9,11-Hexadecadienal | (E)-8-Heptadecen-1-ol |
| (Z,E)-9,11-Hexadecadienal | (E)-8-Heptadecenyl acetate |
| (Z,Z)-9,11-Hexadecadienal | (Z)-8-Heptadecen-1-ol |
| (Z)-9-Heptadecenal | (E,E)-5,9-Octadecadien-1-ol |
| (E)-10-Heptadecenyl acetate | (E,E)-5,9-Octadecadienyl acetate |
| (Z)-11-Heptadecen-1-ol | (E,E)-9,12-Octadecadien-1-ol |
| (Z)-11-Heptadecenyl acetate | (Z,Z)-9,12-Octadecadienyl acetate |
| (E,E)-4,8-Heptadecadienyl acetate | (Z,Z)-9,12-Octadecadienal |
| (Z,Z)-8,10-Heptadecadien-1-ol | (Z,Z)-11,13-Octadecadienal |
| (Z,Z)-8,11-Heptadecadienyl acetate | (E,E)-11,14-Octadecadienal |
| (E)-2-Octadecenyl acetate | (Z,Z)-13,15-Octadecadienal |
| (E)-2-Octadecenal | (Z,Z,Z)-3,6,9-Octadecatrienyl acetate |
| (Z)-2-Octadecenyl acetate | (E,E,E)-9,12,15-Octadecatrien-1-ol |
| (Z)-2-Octadecenal | (Z,Z,Z)-9,12,15-Octadecatrienyl |
| | acetate |
| (E)-9-Octadecen-1-ol | (Z,Z,Z)-9,12,15-Octadecatrienal |
| (E)-9-Octadecenyl acetate | |
| (E)-9-Octadecenal | |
| (Z)-9-Octadecen-1-ol | |
| (Z)-9-Octadecenyl acetate | |
| (Z)-9-Octadecenal | |
| (E)-11-Octadecen-1-ol | |
| (E)-11-Octadecenal | |
| (Z)-11-Octadecen-1-ol | |
| (Z)-11-Octadecenyl acetate | |
| (Z)-11-Octadecenal | |
| (E)-13-Octadecenyl acetate | |
| (E)-13-Octadecenal | |
| (Z)-13-Octadecen-1-ol | |
| (Z)-13-Octadecenyl acetate | |
| (Z)-13-Octadecenal | |
| (E)-14-Octadecenal | |
| (E,Z)-2,13-Octadecadien-1-ol | |
| (E,Z)-2,13-Octadecadienyl acetate | |
| (E,Z)-2,13-Octadecadienal | |
| (Z,E)-2,13-Octadecadienyl acetate | |
| (Z,Z)-2,13-Octadecadien-1-ol | |
| (Z,Z)-2,13-Octadecadienyl acetate | |
| (E,E)-3,13-Octadecadienyl acetate | |
| (E,Z)-3,13-Octadecadienyl acetate | |
| (E,Z)-3,13-Octadecadienal | |
| (Z,E)-3,13-Octadecadienyl acetate | |
| (Z,Z)-3,13-Octadecadienyl acetate | |
| (Z,Z)-3,13-Octadecadienal | |

As used herein, “pheromone biosynthesis-activating neuropeptide” or “PBAN” refer to a neurohormone produced by a cephalic organ, the subesophageal ganglion. PBAN stimulates sex pheromone biosynthesis in the pheromone gland via an influx of extracellular Ca²⁺.

As used herein, “plant incorporated” refers to being in or a part of the plant by genetic modification. In one embodiment, a plant incorporated insecticide comprises an insecticide that is produced by a plant which has been engineered with a recombinant transgene coding for the insecticide. In a particular embodiment, a plant can be engineered to express a crystal protein (cry protein) from the spore forming bacterium Bacillus thuringiensis (Bt). The cry protein is toxic to many species of insects. In another embodiment, a plant can be engineered to express a nucleic acid-based insecticide, which when ingested by the insect, causes downregulation of a target gene in the insect essential for growth, reproduction or survival (see, e.g., U.S. Pat. No. 8,759,306).

As used herein, “plant species” refers to a group of plants belonging to various officially named plant species that display at least some sexual compatibility amongst themselves.

As used herein, “recombinant” broadly describes various technologies whereby genes can be cloned, DNA can be sequenced, and protein products can be produced. As used herein, the term also describes proteins that have been produced following the transfer of genes into the cells of plant host systems.

As used herein, “RNAi-based insecticide” or “RNAi-based pesticide” refers to the use of RNA interference for pest control. Double-stranded RNA (dsRNA) or small interfering (siRNA) can be produced by a transgenic plant engineered to express the dsRNA or siRNA. Alternatively, the dsRNA or siRNA can be synthesized in vitro or produced in bacteria. If produced in vitro or in bacteria, the dsRNA or siRNA can then be formulated into a spray and applied to plants for pest control.

As used herein, “semiochemicals” refer to chemicals (scents, odors, tastes, pheromones, pheromone-like compounds, or other chemosensory compounds) that mediate interactions between organisms. These chemicals can modify behavior of the organisms.

As used herein, “synthetic pheromone” or “synthetic pheromone composition” refers to a chemical composition of one or more specific isolated pheromone compounds. Typically, such compounds are produced synthetically and mimic the response of natural pheromones. In some embodiments, the behavioral response to the pheromone is attraction. In other embodiments, the species to be influenced is repelled by the pheromone.

As used herein, the term “synthetically derived” when used in reference to a chemical compound is intended to indicate that the referenced chemical compound is transformed from starting material to product by human intervention. In some embodiments, a synthetically derived chemical compound can have a chemical structure corresponding to an insect pheromone which is produced by an insect species.

As used herein, the term “synergistic” or “synergistic effect” obtained by the taught methods can be quantified according to Colby's formula (i.e. (E)=X+Y−(X*Y/100). See Colby, R. S., “Calculating Synergistic and Antagonistic Responses of Herbicide Combinations,” 1967 Weeds, vol. 15, pp. 20-22, incorporated herein by reference in its entirety. Thus, by “synergistic” is intended a component which, by virtue of its presence, increases the desired effect by more than an additive amount.

As used herein, “transgene” refers to a gene that will be or is inserted into a host genome, comprising a protein coding region to express a protein or a nucleic acid region to downregulate a target gene in the host.

As used herein, “transgenic plant” refers to a genetically modified plant which contains at least one transgene.

As used herein, “transgenic insecticidal trait” refers to a trait exhibited by a plant that has been genetically engineered to express a nucleic acid or polypeptide that is detrimental to one or more pests. In one embodiment, the trait comprises the expression of vegetative insecticidal proteins (VIPs) from Bacillus thuringiensis, lectins and proteinase inhibitors from plants, terpenoids, cholesterol oxidases from Streptomyces spp., insect chitinases and fungal chitinolytic enzymes, bacterial insecticidal proteins and early recognition resistance genes. In another embodiment, the trait comprises the expression of a Bacillus thuringiensis protein that is toxic to a pest. In one embodiment, the Bt protein is a Cry protein (crystal protein). Bt crops include Bt corn, Bt cotton and Bt soy. Bt toxins can be from the Cry family (see, for example, Crickmore et al., 1998, Microbiol. Mol. Biol. Rev. 62: 807-812), which are particularly effective against Lepidoptera, Coleoptera and Diptera. Examples of genes coding for Bt proteins include: Cry1A, cry1Aa1, cry1Aa2, cry1Aa3, cry1Aa4, cry1Aa5, cry1Aa6, cry1Aa7, cry1Aa8, cry1Aa9, cry1Aa10, cry1Aa11, cry1Ab1, cry1Ab2, cry1Ab3, cry1Ab4, cry1Ab5, cry1Ab6, cry1Ab7, cry1Ab8, cry1Ab9, cry1Ab10, cry1Ab11, cry1Ab12, cry1Ab13, cry1Ab14, cry1Ac1, cry1Ac2, cry1Ac3, cry1Ac4, cry1Ac5, cry1Ac6, cry1Ac7, cry1Ac8, cry1Ac9, cry1Ac10, cry1Ac11, cry1Ac12, cry1Ac13, cry1Ad1, cry1Ad2, cry1Ae1, cry1Af1, cry1Ag1, cry1B, cry1Ba1, cry1Ba2, cry1Bb1, cry1Bc1, cry1Bd1, cry1Be1, cry1C, cry1Ca1, crylCa2, crylCa3, crylCa4, crylCa5, crylCa6, crylCa7, cry1Cb1, cry1Cb2, cry1D, cry1Da1, cry1Da2, cry1Db1, cry1E, cry1Ea1, cry1Ea2, cry1Ea3, cry1Ea4, cry1Ea5, cry1Ea6, cry1Eb1, cry1F, cry1Fa1, cry1Fa2, cry1Fb1, cry1Fb2, cry1Fb3, cry1Fb4, cry1G, cry1Ga1, cry1Ga2, cry1Gb1, cry1Gb2, cry1H, cry1Ha1, cry1Hb1, cry11, cry11a1, cry11a2, cry11a3, cry11a4, cry11a5, cry11a6, cry11b1, cry11c1, cry11d1, cry11e1, cry1l-like, cry1J, cry1Ja1, cry1Jb1, cry1Jc1, cry1Ka1, cry1-like, cry2A, cry2Aa1, cry2Aa2, cry2Aa3, cry2Aa4, cry2Aa5, cry2Aa6, cry2Aa7, cry2Aa8, cry2Aa9, cry2Ab1, cry2Ab2, cry2Ab3, cry2Ac1, cry2Ac2, cry2Ad1, cry3A, cry3Aa1, cry3Aa2, cry3Aa3, cry3Aa4, cry3Aa5, cry3Aa6, cry3Aa7, cry3B, cry3Ba1, cry3Ba2, cry3Bb1, cry3Bb2, cry3Bb3, cry3Ca1, cry4Aa1, cry4Aa2, cry4Ba1, cry4Ba2, cry4Ba3, cry4Ba4, cry5Aa1, cry5Ab1, cry5Ac1, cry5Ba1, cry6Aa1, cry6Ba1, cry7Aa1, cry7Ab1, cry7Ab2, cry8Aa1, cry8Ba1, cry8Ca1, cry9Aa1, cry9Aa2, cry9Ba1, cry9Ca1, cry9Da1, cry9Da2, cry9Ea1, cry9 like, cry10Aa1, cry10Aa2, cry11Aa1, cry11Aa2, cry11Ba1, cry11Bb1, cry12Aa1, cry13Aa1, cry14Aa1, cry15Aa1, cry16Aa1, cry17Aa1, cry18Aa1, cry18Ba1, cry18Ca1, cry19Aa1, cry19Ba1, cry20Aa1, cry21Aa1, cry21Aa2, cry22Aa1, cry23Aa1, cry24Aa1, cry25Aa1, cry26Aa1, cry27Aa1, cry28Aa1, cry28Aa2, cry29Aa1, cry30Aa1, cry31Aa1, cry34, cry35, cyt1Aa1, cyt1Aa2, cyt1Aa3, cyt1Aa4, cyt1Ab1, cyt1Ba1, cyt2Aa1, cyt2Ba1, cyt2Ba2, cyt2Ba3, cyt2Ba4, cyt2Ba5, cyt2Ba6, cyt2Ba7, cyt2Ba8, cyt2Bb1, VIP3A.

As used herein, “volatile compounds” refers to organic compounds or materials that are vaporizable at ambient temperature and atmospheric pressure without the addition of energy by some external source. Any suitable volatile compound in any form may be used. Volatile liquids composed of a single volatile compound are preferred for large-scale application, but volatile solids can also be used for some specialized applications. Liquids and solids suitable for use may have more than one volatile component, and may contain non-volatile components. The volatile compounds may be commercially pure or blended and, furthermore, may be obtained from natural or synthetic sources.

As used herein, the terms “resistant”, “resistance”, or “pest resistance” refers to the following. Resistance is caused by genes in the target insect that reduces susceptibility to a toxin, and is a trait of an individual. Resistance is defined as a phenotype of an individual that can survive on the transgenic insecticidal plant from egg to adult and produce viable offspring. For Bt toxins expressed in crops, this means that an individual must grow and mature feeding only on the Bt crop, and then mate and produce viable offspring. There is much confusion in the scientific literature over the definition of resistance. However, from a genetic or an evolutionary perspective, it is essential to define resistance as a trait of an individual. A consequence of this definition is that if only one individual in a population is resistant, the population contains resistance (Andow 2008).

As used herein, the term “cross-resistance” refers to resistance to all pesticidal compounds in the same sub-group that share a common mode of action.

As used herein, the term “refuge” refers to a habitat in which the target pest can maintain a viable population in the presence of Bt crop fields, where there is no additional selection for resistance to Bt toxins and insects occur at the same time as in the Bt fields (Ives and Andow, 2002). Refuges can be structured (deliberately planted in association with the Bt crop) or unstructured (naturally present as part of the cropping system). The refuge can comprise the non-Bt crop, another crop that is a host for the target pest or pests, or wild host plants. The refuge can be managed to control pest damage, as long as the control methods do not reduce the population to such low levels that susceptible populations are driven to extirpation (Ives and Andow, 2002). The effectiveness of any refuge will depend on its size and spatial arrangement relative to the Bt crop, the behavioral characteristics (movement, mating) of the target pests and the additional management requirements of the refuge.

As used herein, the term “susceptible” is used herein to refer to an insect having no or virtually no resistance to an insecticidal trait or a chemical insecticide. The term “susceptible” is therefore equivalent to “non-resistant”.

As used herein, the term “field plot” refers to any situation where plants are grown together in a contiguous physical area. Examples of such field plots include but are not limited to monoculture, plantations, range lands, golf courses, forests, vineyards, orchards, nurseries, row crops, and plants grown under a central pivot irrigation system. The systems and methods of the present invention can be applied to any way of growing plants, including but not limited to minimized tilling, zero or no-tilling, organic, non-organic, ploughed, harrowed, hoed, irrigated, non-irrigated, dry land, row plantings, hill plantings, plants grown from seed, plants grown from cuttings, plants grown from tissue culture, plants grown from rhizomes, plants grown from tubers and plants grown from bulbs.

As used herein, the term “farm” refers to an area of land and its buildings used for growing crops and rearing animals. Land on a farm may be cultivated for the purpose of agricultural production, and “farming” refers to making a living by growing crops or keeping livestock.

The present invention provides a method of reducing or preventing plant damage in a field plot which comprises plants of a plant population, wherein the entire field plot further comprises one or more pests capable of damaging the plants, said method comprising: a. applying a mating disruption tactic to the entire field plot, wherein said mating disruption tactic is capable of disrupting the mating of the one or more pests; and b. disrupting the expression of one or more target genes in the one or more pests, wherein said disruption of the one or more target genes enhances mating disruption, wherein said method reduces or prevents plant damage from the one or more pests as a result of the applications when compared to a control field plot which only had one or none of the applications. In some embodiments, applying a mating disruption tactic comprises applying one or more pheromones or pheromone blends. In other embodiments, the one or more pheromones or pheromone blends comprises one or more pheromones listed in Table 2. In some preferred embodiments, the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

In some embodiments, applying a mating disruption tactic comprises spraying one or more pheromones or pheromone blends in the field plot. In other embodiments, the one or more pheromones or pheromone blends comprises one or more pheromones listed in Table 2. In some preferred embodiments, the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

In some embodiments, applying a mating disruption tactic comprises emitting one or more pheromones or pheromone blends from one or more dispensers placed in the field plot. In other embodiments, the one or more pheromones or pheromone blends comprises one or more pheromones listed in Table 2. In some preferred embodiments, the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

In some embodiments, applying a mating disruption tactic comprises spraying one or more pheromones or pheromone blends in the field plot, and disrupting expression of one or more target genes comprises feeding dsRNA to the one or more pests. In a preferred embodiment, the dsRNA fed to the one or more pests are infused in phagostimulants. In some embodiments, applying a mating disruption tactic comprises spraying one or more pheromones or pheromone blends in the field plot, and disrupting expression of one or more target genes comprises spraying RNAi molecules in the field plot. In a preferred embodiment, the RNAi molecules are siRNA or dsRNA infused in phagostimulants. In some embodiments, applying a mating disruption tactic comprises scattering pheromone- or pheromone blend-coated granules in the field plot, and disrupting expression of one or more target genes comprises growing transgenic plants expressing RNAi molecules in the field plot as a source of food for the one or more pests.

In one embodiment, the target gene comprises one or more pheromone biosynthesis-activating neuropeptides (PBANs) in the one or more pests. In another embodiment, disrupting one or more PBANs makes the mating disruption more effective. In another embodiment, disrupting one or more PBANs comprises disrupting by RNA interference. In another embodiment, each PBAN is from a pest of the same species as each pest damaging the plants.

In some embodiments, the target gene comprises: chromatin-remodeling ATPases, prothoraciotropic hormone, molt-regulating transcription factors 3, eclosion hormone precursor, p450 monooxygenase, allatoregulating neuropeptides, 3-hydroxy-3-methylglutaryl coenzyme A reductase (HMGR), vacuolar-type H+-ATPases, chitinases, PCGP, arf1, arf2, tubulins, cullin-1, acetylcholine esterases, β1 integrins, iron-sulfur proteins, aminopeptidaseN, arginine kinases, chitin synthases, or any combination thereof, in the one or more pests.

In one embodiment, the one or more target genes comprises one or more genes associated with oviposition. In another embodiment, the genes associated with oviposition are selected from the group consisting of an allatoregulating neuropeptide, a GSK-3 gene, an EMP24/GP25 gene, a chemosensory protein gene, a subolesin/akirin transcription factor gene, an HMG-CoA reductase gene, a purity-of-essence gene, a glucose dehydrogenase gene, a neurocalcin homologue gene, a Scavenger receptor class B member 1 gene, an acyl-CoA delta-11-desaturase gene, a bcl-2-related ovarian killer gene, a ubiquinone biosynthesis gene and an odorant receptor gene.

In one embodiment, the mating disruption tactic is used to control one pest and the disruption in expression of one or more target genes is used to control another pest. In one embodiment, said mating disruption tactic is capable of disrupting the mating of a lepidopteran pest. In another embodiment, the target gene is from a sucking pest. In a further embodiment, the sucking pest is a pentatomid. In yet another embodiment, the sucking pest is a stink bug.

The present invention provides a method of reducing or preventing plant damage in a field plot which comprises plants of a plant population, wherein the entire field plot further comprises one or more pests capable of damaging the plants, said method comprising: applying an attract-and-kill tactic to the entire field plot, wherein said attract-and-kill tactic comprises applying one or more semiochemicals or factors and disrupting expression of one or more target genes in one or more pests, wherein said disruption is capable of killing the one or more pests, wherein said method reduces or prevents plant damage from the one or more pests as a result of the application when compared to a control field plot which did not have the application. In one embodiment, the one or more semiochemicals comprise one or more pheromones or pheromone blends. In other embodiments, the one or more pheromones or pheromone blends comprises one or more pheromones listed in Table 2. In some preferred embodiments, the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

In some embodiments, applying one or more semiochemicals or factors comprises emitting one or more pheromones or pheromone blends from one or more dispensers placed in one or more traps in the field plot. In other embodiments, the one or more pheromones or pheromone blends comprises one or more pheromones listed in Table 2. In some preferred embodiments, the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof. In some embodiments, applying one or more semiochemicals or factors comprises emitting one or more pheromones or pheromone blends from one or more dispensers placed in one or more traps in the field plot, and wherein disrupting expression of one or more target genes comprises feeding dsRNA to the one or more pests.

In one embodiment, the reduction in crop damage comprises a decrease in one or more populations of pests in the entire field plot.

In another embodiment, the one or more target genes comprises one or more genes associated with lethality or reduced growth when the gene is down regulated. In another embodiment, the genes associated with lethality or reduced growth when down regulated are selected from the group consisting of a chitinase gene, a cytochrome P450 monooxygenase gene, a vacuolar-type Ht ATPase gene, a chromatin remodelling ATPase gene, a prothoraciotropic hormone gene, a molt-regulating transcription factors 3 gene, a eclosion hormone precursor gene, a chitin synthase gene, PGCP gene, a tubulin gene, an arf gene, a trehalose phosphate synthase gene, a ribosomal protein gene, a beta-actin gene, a protein transport gene, a coatomer subunit gene, a cullin gene, a chitinase gene, an acetylcholinesterase gene, a β1 integrin gene, an iron-sulfur protein gene, an aminopeptidaseN gene, an arginine kinase gene and a proteasome-associated gene.

In another embodiment, the one or more semiochemicals or factors comprise one or more attractants. In another embodiment, the one or more attractants comprise one or more host plant chemical, non-host plant chemical, synthetic volatile chemical, or natural volatile chemical. In another embodiment, the one or more attractants are identified through binding to one or more pest odorant binding proteins. In another embodiment, the one or more attractants comprise one or more host plant volatile chemical. In another embodiment, the one or more host plant volatile chemical comprise heptanal or benzaldehyde. In another embodiment, the one or more attractants comprise one or more male pheromones. In another embodiment, the one or more attractants comprise one or more ovipositioning pheromones. In another embodiment, the one or more attractants comprise one or more female attractants. In another embodiment, the one or more female attractants comprise ethylene. In another embodiment, the one or more attractants comprise one or more kairomones.

In some embodiments, disrupting the expression of one or more target genes in the one or more pests comprises RNA interference (RNAi). In further embodiments, the RNAi comprises one or more double-stranded RNA, one or more small interfering RNA (siRNA), or a combination thereof. In some embodiments, the one or more double-stranded RNA, one or more small interfering RNA (siRNA), or a combination thereof, are expressed in a plant. In other embodiments, the one or more double-stranded RNA, one or more small interfering RNA (siRNA), or a combination thereof, are formulated for a broadcast spray, a feeding station, a food trap, or any combination thereof.

In some embodiments, the one or more pests comprises one or more sucking pests. In some embodiments, the one or more pests is a member of the class Insecta. In further embodiments, the one or more pests is a member of the order Lepidoptera. In some embodiments, the one or more pests is a member of the order Hemiptera. In some embodiments, the one or more pests is a member of the family Noctuidae. In further embodiments, the one or more pests is a member of the family Pentatomidae. In some embodiments, the one or more pests is a member of the order Coleoptera. In further embodiments, the one or more pests is a member of the family Curculionidae. In some embodiments, the one or more pests is a member of a genus selected from the group consisting of: Helicoverpa, Spodoptera, Euschistus, Anthonomus and Nezara, or any combination thereof. In further embodiments, the one or more pests is a species selected from the group consisting of: Helicoverpa zea, Helicoverpa armigera, Spodoptera frupperda, Spodoptera cosmioides, Euschistus heros, Anthonomus grandis and Nezara viridula, or any combination thereof.

As used herein, the term “plant damage” refers to any destruction or loss in value, usefulness, or ability resulting from an action or event associated with a pest such as an insect. Types of plant damage include, but are not limited to, the following. Feeding damage occurs as a result of direct feeding on above-ground and/or below-ground plant parts. Holes or notches in foliage and other plant parts, leaf skeletonizing (removal of tissue between the leaf veins), leaf defoliation, cutting plants off at the soil surface, or consumption of roots can all occur from pests with chewing mouthparts. Chewing pests can also bore or tunnel into plant tissue. Stem-boring insects can kill or deform individual stems or whole plants. Leaf mining insects feed between the upper and lower surfaces of leaves, creating distinctive tunnel patterns visible as translucent lines or blotches on leaves. Pests with sucking mouthparts can suck sap from plant tissue, which may cause spotting or stippling of foliage, leaf curling and stunted or misshapen fruits. Insects such as thrips have rasping mouthparts that scrape the surface of foliage or flower parts, disrupting plant cells. Oviposition damage occurs as a result of egg laying into plant tissue. Heavy oviposition into stems can cause death or dieback of stems or branches on the plant. Flagging is a result of dieback of the ends of stems or branches. Oviposition in fruits can result in misshapen or aborted fruits, and is sometimes called cat-facing. Some insects form galls on their host plant, causing the plant to grow abnormally. Depending on the insect species, the gall formation can be stimulated by feeding or by oviposition into plant tissue. Pests can also cause damage by transmitting plant pathogens such as viruses, fungi, bacteria, mollicutes, protozoa, and nematodes. The transmission can be accidental or incidental (the plant pathogen enters plant tissue through feeding or oviposition wounds), phoretic or passive (the pest carries the plant pathogen from one plant to another), or active (the plant pathogen is carried within the body of the pest, and a plant is inoculated with the pathogen when the pest feeds on a plant).

As used herein, the term “plant symptom” refers to any abnormal states that indicate a bodily disorder. The plant symptom can be visible or not visible. Examples of plant symptoms include, but are not limited to: presence of pests in plant parts; poor stand or germination; wilted or lodged plants; roots severed or damaged; stalks with puncture holes; plants not emerged; plants cut off at or below ground; stunted plants; physically distorted plants; plants with odd colors; larvae in soil at or near roots; holes in leaves; irregular pieces of leaves missing from edges and/or center of leaves; tunneling or boring in leaves; mottled leaves; reduced leaf area; leaf defoliation; leaves discolored; dying leaves; tunneling or boring in stalks; distorted or broken stalks; dying stalks; distorted fruit; reduced fruit production. As an example, for corn, the ear, tassels, silks, husks, whorls and kernels can all have symptoms of pest damage, such as: anthers on tassel with pieces missing; whorls containing pests; distorted ear; larvae in ear; short, thread-like or small particle frass (debris or excrement from pest) in silk or on surrounding husk; numerous silks clipped off; silks often matted, discolored, and damp in silk channel or at ear tip; husks with round or oval holes often penetrating into ear; husks with irregular holes; dry, highly structured, pillow-shaped frass present on plants and on ground; kernels with chewing damage; kernels punctured through husk are sunken or popped.

As used herein, “signs of plant damage” or “signs of damage” refer to any plant symptoms that can be observed and indicate that the plant has been negatively affected by a pest compared to a plant that has not been affected by a pest or is resistant to a pest.

A plant, line or cultivar that shows fewer or reduced symptoms to a biotic pest or pathogen than a susceptible (or more susceptible) plant, line or variety to that biotic pest or pathogen has resistance or is resistant to said pest or pathogen. In one embodiment, resistant plants show no symptoms. In another embodiment, resistant plants show some symptoms but are still able to produce marketable product with an acceptable yield. Some lines that are referred to as resistant are only so in the sense that they may still produce a crop, even though the plants may appear visually stunted and the yield is reduced compared to uninfected plants. As defined by the International Seed Federation (ISF), a non-governmental, non-profit organization representing the seed industry (see “Definition of the Terms Describing the Reaction of Plants to Pests or Pathogens and to Abiotic Stresses for the Vegetable Seed Industry”, May 2005), the recognition of whether a plant is affected by or subject to a pest or pathogen can depend on the analytical method employed. Plant resistance is defined by the ISF as the ability of plant types to restrict the growth and development of a specified pest or pathogen and/or the damage they cause when compared to susceptible plant varieties under similar environmental conditions and pest or pathogen pressure. Resistant plant types may still exhibit some disease symptoms or damage. Two levels of plant resistance are defined. The term “high/standard resistance” is used for plant varieties that highly restrict the growth and development of the specified pest or pathogen under normal pest or pathogen pressure when compared to susceptible varieties. “Moderate/intermediate resistance” is applied to plant types that restrict the growth and development of the specified pest or pathogen, but exhibit a greater range of symptoms or damage compared to plant types with high resistance. Plant types with intermediate resistance will show less severe symptoms than susceptible plant varieties, when grown under similar field conditions and pathogen pressure. Methods of evaluating resistance are well known to one skilled in the art. Such evaluation may be performed by visual observation of a plant or a plant part (e.g., leaves, roots, flowers, fruits et al.) in determining the severity of symptoms. For example, when each plant is given a resistance score on a scale of 1 to 5 based on the severity of the reaction or symptoms, with 1 being the resistance score applied to the most resistant plants (e.g., no symptoms, or with the least symptoms), and 5 the score applied to the plants with the most severe symptoms, then a line is rated as being resistant when at least 75% of the plants have a resistance score at a 1, 2, or 3 level, while susceptible lines are those having more than 25% of the plants scoring at a 4 or 5 level. If a more detailed visual evaluation is possible, then one can use a scale from 1 to 10 so as to broaden out the range of scores and thereby hopefully provide a greater scoring spread among the plants being evaluated.

A tolerant plant may exhibit a phenotype wherein symptoms of damage remain mostly if not totally absent upon exposure of said plant to a pest infestation.

A susceptible or non-resistant plant has no or virtually no resistance to a pest.

In one embodiment, applying a mating disruption tactic comprises applying one or more pheromones. In another embodiment, the one or more pheromones comprise sprayable formulations or are in aerosol emitters or hand applied dispensers.

A pheromone is a chemical substance that is usually produced by an animal or insect and serves especially as a stimulus to other individuals of the same species for one or more behavioral responses. Pheromones can be used to disrupt mating of invading insects by dispensing the pheromones or the pheromone scent in the air, so the males cannot locate the females, which disrupts the mating process. Pheromones can be produced by the living organism, or artificially produced. This pest control method does not employ insecticides, so the use of pheromones is safer for the environment and for living organisms.

Sex pheromones are used in the chemical communication of many insects for attracting the species of the opposite sex to engage in reproduction. Pheromones are useful for pest control largely through four means: monitoring, mass trappings, attract-and-kill, and disruption or impairment of communication. The “monitoring” methodology attracts the pest to a central area, which allows the grower to obtain precise information on the size of the pest population in order to make informed decisions on pesticide use or non-use. “Mass trappings” brings the pest to a common area and physically traps it, which hinder production of new generations of the pest. “Attract-and-kill” allows the pest to be drawn into a centrally located container and killed in the container by a pesticide, reducing the need to spread pesticides in broad areas. “Disruption of communication” can occur in that a large concentration of sex pheromone can mask naturally occurring pheromones or saturate the receptors in the insect causing impairment of communication and disruption of natural reproductive means. For each one of these means, each individual species of pest needs to be treated with a tailor-made composition.

Mating disruption is a pest control technology that works by placing enough artificial sources of pheromone in an area so that the probability of a female being found by a male, mating, and laying viable eggs is reduced below the point where economically significant damage occurs. Mating disruption pheromone systems are available for the codling moth, Oriental fruit moth, dogwood borer, peachtree borer and lesser peachtree borer as well as for some leafroller species. These are used extensively in western states and a number of growers are using them in the eastern seaboard.

Mating disruption has many advantages as a pest control method. It is environmentally friendly, with negligible health risks to applicator and consumer; highly selective to the pest species being targeted for disruption and non-target effects are not observed; no documented cases of resistance to the pheromone itself; and reduced worker re-entry into the field after application and shorter preharvest intervals.

Mating disruption using female sex pheromones operates via modulating the behaviour of adult males, in so far as trap catch shutdown is a property of males only. Trap catch shutdown is used as proxy for indicating that no mating has occurred in the field. It is important to realize that adult moths cause negligible damage because they only feed from nectar and, for some species, they do not feed at all. Thus, damage is a property of the females, whose progeny of caterpillars will attack the host crop.

Mating disruption, especially when only partially successful, may benefit from synergies with other pest control technologies. In one embodiment, mating disruption is combined with RNA interference (see below) for more effective control of the same or different pests. In one embodiment, the mating disruption tactic is used to control one pest and the disruption in expression of one or more target genes is used to control another pest. In one embodiment, said mating disruption tactic is capable of disrupting the mating of a lepidopteran pest. In another embodiment, the target gene is from a sucking pest.

### PBAN as Target for RNAi

In one embodiment of the present invention, the efficacy of mating disruption can be increased by using RNA interference (RNAi) technology to hinder the expression of the pheromone biosynthesis-activating neuropeptide (PBAN) (Choi, M-Y et al. (2012) Phenotypic impacts of PBAN RNA interference in an ant, Solenopsis Invicta, and a moth, Helicoverpa zea. Journal of Insect Physiology 58: 1159-1165). PBAN stimulates production of the female sex pheromone in female virgins. Thus, the disruption in expression of PBAN reduces the calling ability of females. PBAN RNAi can be fed to larva, where it decreases growth rate and can impede development of larva to pupa. Those female larvae that do mature to adulthood, have decreased amounts of sex pheromone (Targeting Pheromones in Fire Ants. Agricultural Research. 2014. 6). The 18-amino acid residue PBAN for H. zea has been characterized (Raina, A. K., et al. A pheromonotropic peptide of Helicoverpa zea, with melanizing activity, interaction with PBAN, and distribution of immunoreactivity. Arch. Insect Biochem. Physiol. 53, 147-57 (2003)). In one embodiment, the method comprises applying a mating disruption tactic and disrupting one or more pheromone biosynthesis-activating neuropeptides (PBANs) in the one or more population of pests. In another embodiment, disrupting one or more PBANs makes the mating disruption more effective. In another embodiment, disrupting one or more PBANs comprises disrupting by RNA interference.

Host Finding and/or Oviposition Genes as Targets for RNAi

In another embodiment of the present invention, the efficacy of mating disruption can be enhanced by using RNA interference (RNAi) technology to hinder the expression of genes important for host finding and/or egg laying patterns. For example, proteins that play a role in oviposition include: GSK-3, a Ser/Thr kinase (Fabres, A. et al. (2010) Effect of GSK-3 activity, enzymatic inhibition and gene silencing by RNAi on tick oviposition and egg hatching. Parasitology 137: 1537-1546); logjam, a predicted protein homologous to EMP24/GP25 transmembrane components of cytoplasmic vesicles (Carney, G. E. and Taylor, B. J. (2003) logjam encodes a predicted EMP24/GP25 protein that is required for Drosophila oviposition behavior. Genetics 164: 173-186); chemosensory protein (Gong, L. et al. (2012) Cloning and characterization of three chemosensory proteins from Spodoptera exigua and effects of gene silencing on female survival and reproduction. Bulletin of Entomological Research 102(5): 600-609); subolesin/akirin transcription factors (Smith, A. et al. (2009) The impact of RNA interference of the subolesin and voraxin genes in male Amblyomma hebraeum (Acari: Ixodidae) on female engorgement and oviposition. Exp. Appl. Acarol. 47: 71-86, Moreno-Cid, J. A. et al. (2013) Control of multiple arthropod vector infestations with subolesin/akirin vaccines. Vaccine 31: 1187-1196); 3-hydroxy-3-methylglutaryl coenzyme A reductase (HMG-CoA reductase), a key enzyme in the mevalonate pathway (Wang, Z. et al. (2013) RNAi silencing of the HaHMG-CoA reductase gene inhibits oviposition in the Helicoverpa armigera cotton bollworm. PLoS ONE 8(7):e67732). A number of other candidate target genes that are overexpressed in ovipositing female wasps, such as purity-of-essence (large membrane protein containing two zinc finger domains), glucose dehydrogenase (GLD), neurocalcin homologue, Scavenger receptor class B member 1, acyl-CoA delta-11-desaturase,bcl-2-related ovarian killer and a ubiquinone biosynthesis gene, have been identified by transcriptomic experiments (Pannebakker, B. A. et al. (2013) The transcriptomic basis of oviposition behaviour in the parasitoid wasp Nasonia vitripennis. PLoS ONE 8(7): e68608). As olfaction is important for locating oviposition sites and host seeking behavior, identifying genes that are differentially expressed in antennae versus non-olfactory tissues may provide other target genes that are important for host finding and/or egg laying patterns (Leal, W. S. et al. (2013) Differential expression of olfactory genes in the southern house mosquito and insights into unique odorant receptor gene isoforms. PNAS 110(46): 18704-18709). Indeed, the down-regulation of a non-conventional odorant receptor in the beetle pest Phyllotreta striolata impaired the host-plant preferences of P. striolata for cruciferous vegetables (Zhao, Y. Y. et al. (2011) PsOr1, a potential target for RNA interference-based pest management. Insect Mol Biol 20(1): 97-104).

### RNA Interference

Techniques which can be employed in accordance with the present invention to knock down gene expression, include, but are not limited to: (1) disrupting a gene's transcript, such as disrupting a gene's mRNA transcript; (2) disrupting the function of a polypeptide encoded by a gene, or (3) disrupting the gene itself.

For example, antisense RNA, ribozyme, dsRNAi, RNA interference (RNAi) technologies can be used in the present invention to target RNA transcripts of one or more genes of interest, e.g. PBAN genes. Antisense RNA technology involves expressing in, or introducing into, a cell an RNA molecule (or RNA derivative) that is complementary to, or antisense to, sequences found in a particular mRNA in a cell. By associating with the mRNA, the anti sense RNA can inhibit translation of the encoded gene product. The use of antisense technology to reduce or inhibit the expression of an insect gene has been described, for example, in Cabrera et al. (1987) Phenocopies induced with antisense RNA identify the wingless gene, Cell, 50(4): 659-663.

A ribozyme is an RNA that has both a catalytic domain and a sequence that is complementary to a particular mRNA. The ribozyme functions by associating with the mRNA (through the complementary domain of the ribozyme) and then cleaving (degrading) the message using the catalytic domain.

RNA interference (RNAi) is the process of sequence-specific, post-transcriptional gene silencing or transcriptional gene silencing in animals and plants, initiated by double-stranded RNA (dsRNA) that is homologous in sequence to the silenced gene. The RNAi technique is discussed, for example, in Elbashir, et al., Methods Enzymol. 26:199 (2002); McManus & Sharp, Nature Rev. Genetics 3:737 (2002); PCT application WO 01/75164; Martinez et al., Cell 110:563 (2002); Elbashir et al., supra; Lagos-Quintana et al., Curr. Biol. 12:735 (2002); Tuschl et al., Nature Biotechnol. 20:446 (2002); Tuschl, Chembiochem. 2:239 (2001); Harborth et al., J. Cell Sci. 114:4557 (2001); et al., EMBO J. 20:6877 (2001); Lagos-Quintana et al., Science 294:8538 (2001); Hutvagner et al., loc cit, 834; Elbashir et al., Nature 411:494 (2001).

The term “dsRNA” or “dsRNA molecule” or “double-strand RNA effector molecule” refers to an at least partially double-strand ribonucleic acid molecule containing a region of at least about 19 or more nucleotides that are in a double-strand conformation. The double-stranded RNA effector molecule may be a duplex double-stranded RNA formed from two separate RNA strands or it may be a single RNA strand with regions of self-complementarity capable of assuming an at least partially double-stranded hairpin conformation (i.e., a hairpin dsRNA or stem-loop dsRNA). In various embodiments, the dsRNA consists entirely of ribonucleotides or consists of a mixture of ribonucleotides and deoxynucleotides, such as RNA/DNA hybrids. The dsRNA may be a single molecule with regions of self-complementarity such that nucleotides in one segment of the molecule base pair with nucleotides in another segment of the molecule. In one aspect, the regions of self-complementarity are linked by a region of at least about 3-4 nucleotides, or about 5, 6, 7, 9 to 15 nucleotides or more, which lacks complementarity to another part of the molecule and thus remains single-stranded (i.e., the “loop region”). Such a molecule will assume a partially double-stranded stem-loop structure, optionally, with short single stranded 5′ and/or 3′ ends. In one aspect the regions of self-complementarity of the hairpin dsRNA or the double-stranded region of a duplex dsRNA will comprise an Effector Sequence and an Effector Complement (e.g., linked by a single-stranded loop region in a hairpin dsRNA). The Effector Sequence or Effector Strand is that strand of the double-stranded region or duplex which is incorporated in or associates with the RNA induced silencing complex (RISC). In one aspect the double-stranded RNA effector molecule will comprise an at least 19 contiguous nucleotide effector sequence, preferably 19 to 29, 19 to 27, or 19 to 21 nucleotides, which is a reverse complement to the RNA of the target gene. In one embodiment, the RNA is from one or more PBANs (or RNA of oviposition genes or essential genes), or an opposite strand replication intermediate, or the anti-genomic plus strand or non-mRNA plus strand sequences of PBANs (or oviposition sequences or essential gene sequences). One skilled in the art will be able to design suitable double-strand RNA effector molecule based on the nucleotide sequences of PBANs (or oviposition genes or essential genes) in the present invention.

In some embodiments, the dsRNA effector molecule is a “hairpin dsRNA”, a “dsRNA hairpin”, “short-hairpin RNA” or “shRNA”, i.e., an RNA molecule of less than approximately 400 to 500 nucleotides (nt), or less than 100 to 200 nt, in which at least one stretch of at least 15 to 100 nucleotides (e.g., 17 to 50 nt, 19 to 29 nt) is based paired with a complementary sequence located on the same RNA molecule (single RNA strand), and where said sequence and complementary sequence are separated by an unpaired region of at least about 4 to 7 nucleotides (or about 9 to about 15 nt, about 15 to about 100 nt, about 100 to about 1000 nt) which forms a single-stranded loop above the stem structure created by the two regions of base complementarity. The shRNA molecules comprise at least one stem-loop structure comprising a double-stranded stem region of about 17 to about 100 bp; about 17 to about 50 bp; about 40 to about 100 bp; about 18 to about 40 bp; or from about 19 to about 29 bp; homologous and complementary to a target sequence to be inhibited; and an unpaired loop region of at least about 4 to 7 nucleotides, or about 9 to about 15 nucleotides, about 15 to about 100 nt, about 100 to about 1000 nt, which forms a single-stranded loop above the stem structure created by the two regions of base complementarity. It will be recognized, however, that it is not strictly necessary to include a “loop region” or “loop sequence” because an RNA molecule comprising a sequence followed immediately by its reverse complement will tend to assume a stem-loop conformation even when not separated by an irrelevant “stuffer” sequence.

Post-transcriptional gene silencing by RNA interference in insects is similar to that of other eukaryotes. The RNAi-mediated silencing process can be divided into three steps: (1) a long dsRNA expressed or introduced into the cell is digested into small double stranded small non-coding RNAs (either miRNA or siRNA) by the enzyme Dicer; (2) these miRNAs or siRNAs are then unwound and the guide strand is preferentially loaded into the RISC; (3) The RISC, directed by the RNA guide strand, locates mRNAs containing specific nucleotide sequences complementary to the guide, and binds to these sequences to bring about either mRNA target degradation or blockage of translation.

In other eukaryotes dsRNA entering the RNAi pathway are amplified by a host-derived RNA-dependent RNA polymerase (RdRp). However, insects appear to lack an endogenous RdRp. Insects do have transmembrane proteins called SIDs that potentially function in dsDNA uptake, although it is still unclear the extent to which SIDs are involved in insects.

dsRNA or siRNA can be delivered to insects by several ways. dsRNA or siRNA can be introduced into a pest by micro-injection, although this delivery method is only feasible for laboratory settings and not for field pest control. Transgenic plants have been engineered to express dsRNA directed against insect genes (Baum, J. A. et al. (2007) Control of coleopteran insect pests through RNA interference. Nature Biotechnology 25: 1322-1326; Mao, Y. B. et al. (2007) Silencing a cotton bollworm P450 monooxygenase gene by plant-mediated RNAi impairs larval tolerance of gossypol. Nature Biotechnology 25: 1307-1313). RNAi can be triggered in the pest by feeding of the pest on the transgenic plant. Soaking and/or spraying plants with bacteria expressing dsRNA or siRNA is another route (Gan, D. et al. (2010) Bacterially expressed dsRNA protects maize against SCMV infection. Plant Cell Reports 29: 1261-1268).

### Oviposition

Maximum oviposition (51.6 eggs/female) was recorded for H. armigera on a variety of cotton (Gossypium hirsutum LH 900) in a contained field bioassay (Butter, N. S. and Singh, S. (1996) Ovipositional response of Helicoverpa armigera to different cotton genotypes, Phytoparasitica 24(2): 97-102). Torres and Ruberson observed that there were about 0.2-0.4 eggs per cotton plant during peak oviposition season for Heliothis and Helicoverpa cotton bollworms (Tones, J. B. and Ruberson, J. R. (2006) Spatial and temporal dynamics of oviposition behavior of bollworm and three of its predators in Bt and non-Bt cotton fields, Entomologia Experimentalis et Applicata 120: 11-22). An individual gravid female is capable of laying 500 to 3000 eggs, which she deposits singly on leaf hairs and corn silk. Gravid females are therefore capable of ovipositing on many plants within a field. When moth populations are high, several females may lay eggs on a single ear, resulting in 6-8 eggs per sweet corn ear. Given that there can be an average of about one thousand eggs per female, there is inherent asymmetry in mating disruption. The present invention provides methods for dealing with a few mated females that would otherwise be sufficient to infest an entire field if they are not adequately controlled.

### Attractants

Several researchers have shown that host-plant volatile components can serve as attractants (reviewed in: Gregg et al. (2010) Development of a synthetic plant volatile-based attracticide for female noctuid moths. II. Bioassays of synthetic plant volatiles as attractants for the adults of the cotton bollworm, Helicoverpa armigera (Hubner) (Lepidoptera: Noctuidae). Aust. J. Entomol. 49:21-30), and can significantly increase lepidopterans' attraction to sex pheromones when detected in unison (example: Deng et al. (2004) Enhancement of attraction to sex pheromones of Spodoptera exigua by volatile compounds produced by host plants. J. Chem. Ecol 30:2037-2045). Fang and Zhang (2002) demonstrated that in addition to increasing attraction to sex pheromones, host-plant volatiles also positively influence oviposition preference (Fang, Y. and Zhang, Z. (2002) Influence of host-plant volatile components on oviposition behaviour and sex pheromone attractiveness to H. armigera. Acta Entomologica Sinica 45:63-67). Heptanal and benzaldehyde are two host-plant volatile components that significantly increase the attractiveness of an oviposition substrate among mated H. armigera. Additionally, corn silk is a preferred oviposition substrate for Helicoverpa spp., and the concentration of its associated volatile, ethylene, is positively correlated with calling behaviour in virgin female H. zea. Ethylene thus serves as a mating cue and it would logically follow that high concentrations of ethylene would increase the number of locally oviposited eggs (especially considering that effects of mating described below).

In one embodiment, applying an attract-and-kill tactic comprises applying one or more semiochemicals or factors. In one embodiment, the one or more semiochemicals comprise one or more pheromones or pheromone blends. In another embodiment, the one or more semiochemicals or factors comprise one or more attractants. In another embodiment, the one or more attractants comprises one or more host plant chemical, non-host plant chemical, synthetic volatile chemical, or natural volatile chemical. In another embodiment, the one or more attractants are identified through binding to one or more pest odorant binding proteins.

In another embodiment, the one or more attractants comprises one or more host plant volatile chemical. In another embodiment, the one or more host plant volatile chemical comprises heptanal or benzaldehyde. In another embodiment, the one or more attractants comprises one or more female attractants. In another embodiment, the one or more female attractants comprises ethylene.

Jin et al. found that crude extracts of male accessory glands (MAG) stimulated earlier egg maturation (P<0.001) and oviposition (the oviposition ratio was more than 2 times the ratio of the control). (Jin, Z—Y and Gong, H. Male accessory gland derived factors can stimulate oogenesis and enhance oviposition in Helicoverpa armigera (Lepidoptera: Noctuidae). Arch. Insect Biochem. Physiol. 46:175-185, 2001). They also found that proteinaceous components in crude extracts purified by fractionation and sub-fractionation in reverse phase high performance liquid chromatography stimulated earlier egg maturation (P<0.01) and oviposition (more than 2 times the ratio of the control). They called these the oogenesis and oviposition factors (OOSF). The mode of delivery for the OOSFs may involve a vaporization of the molecules in an air-borne spray which has been shown to allow the permeation of PSPs into insect haemolymph (Kennedy, R. Vestaron Corporation, Crops & Chemicals Conference, Raleigh, N.C., July 2015).

In one embodiment, applying an attract-and-kill tactic comprises applying one or more semiochemicals or factors and disrupting expression of a target gene in one or more pests. In one embodiment, the one or more semiochemicals comprise one or more pheromones or pheromone blends. In another embodiment, the one or more semiochemicals or factors comprise oogenesis and oviposition factors (OOSFs). In another embodiment, the OOSFs are applied by vaporization.

### Orthogonality of Olfactory Receptors

Sex pheromones are sensed by dedicated odorant binding proteins (OBPs). This means that in the presence of mating disruption, male OBPs dedicated to sex pheromones are already saturated and female OBPs that sense these molecules may be saturated too. Because host finding and oviposition site selection is sensed by different OBPs, this allows attract-and-kill to occur simultaneously with mating disruption. As an example, an odorant-binding protein (OBP) found in the antennae and seminal fluid of H. armigera and H. assulta is associated with 1-dodecene, a known insect repellent (Sun et al. 2012 Expression in Antennae and Reproductive Organs Suggests a Dual Role of an Odorant-Binding Protein in Two Sibling Helicoverpa Species. PLoS ONE 7(1): e30040 (2012)). OBPs are involved in the perception and release of semiochemicals in insects, and thus this particular OBP may potentially be involved in the detection and delivery of oviposition deterrents. In Spodoptera frugiperda, a trifluoromethyl ketone acts as a pheromone analogue that competitively inhibits the binding of sex pheromones with their associated OBP, and thus reduces pheromone reception in males (Malo et al. 2013 Inhibition of the responses to sex pheromone of the fall armyworm, Spodoptera frupperda. Journal of Insect Science, 13: 134). As these examples show, an understanding of the molecular structures of the odorant binding proteins can lead to novel attractants and repellents which will find use in the methods of the present invention.

### In Silico Screening of Novel Semiochemicals by Docking to OBPs

Computational structure-activity screen of thousands of compounds against OBPs in the target pest can be used to identify new attractants or repellents. See, for example, the work done on fruit fly odor receptors to identify alternative mosquito repellents to DEET (Kain et al. 2013 Odour receptors and neurons for DEET and new insect repellents. Nature, 502: 507-512), which used a high-throughput chemical informatics screen without knowing the 3D crystal structure of the OBP. Thus, for example, structural features shared by compounds demonstrated to be attractive or repellent to mated female pests can be used to screen a vast library of compounds in silico for the presence of these structural features. A training set of known mated female pest attractants or repellents can be assembled to computationally identify a unique subset of descriptors that correlate highly with either attraction or repellency. Also, compounds that may be safe for human use may be identified by applying the in silico screen to an assembled library having chemicals originating from plants, insects or vertebrate species, and compounds already approved for human use.

### Attract-and-Kill Targeted at Females

It is known in the art that noctuid moths, including H. armigera, are attracted to floral scents (Gregg, P. C. et al. Development of a synthetic plant volatile-based attracticide for female noctuid moths. II. Bioassays of synthetic plant volatiles as attractants for the adults of the cotton bollworm, Helicoverpa armigera (Hübner) (Lepidoptera: Noctuidae). Aust. J. Entomol. 49, 21-30 (2010)). It is further known that these floral scents can be mixed with a feeding stimulant (e.g. sugar) and an RNAi in an attract-and-kill formulation. According to the methods of the present invention, these formulations can be field applied to kill both male and female noctuid moths.

In one embodiment of the present invention, the attract-and-kill product combination can be delivered as a broadcast spray.

According to the present invention, mixtures comprising RNAi-based insecticide (produced by bacteria) with or without kairomones and/or ovipositioning pheromones are applied in a field plot, which dramatically reduces crop damage when combined with mating disruption across the field. In one embodiment, the disclosure provides a mixture comprising one or more attractants and one or more RNAi-based insecticides. In another embodiment, the one or more attractants comprises one or more host plant chemical, non-host plant chemical, synthetic volatile chemical or natural volatile chemical. In another embodiment, the one or more attractants comprises one or more male pheromones. In another embodiment, the one or more attractants comprises one or more ovipositioning pheromones. In another embodiment, the one or more attractants comprises one or more female attractants. In another embodiment, the one or more female attractants comprises ethylene. In another embodiment, the one or more attractants comprises one or more kairomones. In another embodiment, the one or more RNAi-based insecticides kills the pest. In yet another embodiment, the pest is a sucking pest. In a further embodiment, the sucking pest is a stink bug.

Gregg et al. (Gregg, P. C. et al. Development of a synthetic plant volatile-based attracticide for female noctuid moths. II. Bioassays of synthetic plant volatiles as attractants for the adults of the cotton bollworm, Helicoverpa armigera (Hübner) (Lepidoptera: Noctuidae). Aust. J. Entomol. 49, 21-30 (2010)) measured the attractiveness of synthetic equivalent of host and non-host plant volatiles to virgin females of H. armigera. A total of 34 different compounds were tested singly and as blends. These compounds included aromatic volatiles found in flowers, such as 2-phenylethanol and phenylacetaldehyde, and volatiles found in leaves, including green leaf volatiles and terpenoids. All of these compounds and their blends are incorporated here in their entirety. The attractiveness of these compounds on mated females was not measured in the Gregg et al. study.

Plant volatiles can be grouped into floral volatiles (fatty acid derivatives, mostly short-chain alcohols and acetates, which are products of nectar fermentation), green leaf volatiles (C6 fatty acid derivatives, straight chain alcohols, aldehydes and esters mostly present in leaves), aromatic compounds (cyclic C6 compounds and their derivatives, found in flowers and leaves) and isoprenoids (mono- and sesquiterpenes which can be found in both leaves and flowers) (Del Socorro, A. P. et al. Development of a synthetic plant volatile-based attracticide for female noctuid moths. I. Potential sources of volatiles attractive to Helicoverpa armigera (Hubner) (Lepidoptera: Noctuidae). Australian Journal of Entomology, 49: 10-20 (2010)).

It is also known that female moths are attracted to ethylene. Therefore, reagents such as ethenphos can be applied in the field to deliver ethylene in situ. Since it is further known that 1-methylcyclopropene (1-MCP) competes with an ethylene binding protein in plants, another aspect of this invention consists of spraying on the border as a way to attract females. Other synthetic volatiles could also work given that non-host volatiles are attractive to Helicoverpa female moths.

Commercial attract and kill products include Magnet® and Noctovi® (ISCA Technologies). Magnet® is a synthetic plant volatile-based attracticide for noctuid pests of agriculture (Del Socorro, A. P. et al. 2010). Noctovi® is an environmentally friendly semiochemical attractant and phagostimulant that can be mixed with insecticides and improves the efficacy and longevity of insecticides.

In one embodiment, applying an attract-and-kill tactic comprises applying one or more semiochemicals or factors and disrupting expression of a target gene in one or more pests. In one embodiment, the one or more semiochemicals comprise one or more pheromones or pheromone blends. In one embodiment, the disruption in expression of the target gene injures or kills the pest. In another embodiment, the one or more pests is a sucking pest. In a further embodiment, the sucking pest is a stink bug.

### Candidate Target Genes to Disrupt for Lethality or Reduced Growth

Target genes whose disruption may lead to lethality or reduced growth of a pest include: chitinase, critically required for insect molting and metamorphosis (Mamta, K. R. et al. (2016) Targeting chitinase gene of Helicoverpa armigera by host-induced RNA interference confers insect resistance in tobacco and tomato. Plant Molecular Biology 90(3): 281-292); cytochrome P450 monooxygenase, V-ATPase and chitin synthase genes (Jin, S. et al. (2015) Engineered chloroplast dsRNA silences cytochrome p450 monooxygenase, V-ATPase and chitin synthase genes in the insect gut and disrupts Helicoverpa armigera larval development and pupation. Plant Biotechnology Journal 13: 435-446); chitinase7, PGCP, chitinase1, ATPase, tubulin1, arf2, tubulin2 and arf1 (Li, H. et al. (2013) Transcriptome analysis and screening for potential target genes for RNAi-mediated pest control of the beet armyworm, Spodoptera exigua. PLoS ONE 8(6):e65931); trehalose phosphate synthase (Chen, J. et al. (2010) Feeding-based RNA interference of a trehalose phosphate synthase gene in the brown planthopper, Nilaparvata lugens. Insect Mol Biol 19: 777-786); ribosomal protein L9 (Upadhyay, S. K. et al. (2011) RNA interference for the control of whiteflies (Bemisia tabaci) by oral route. J Biosci 36: 153-161); β-actin, protein transport protein sec23, coatomer subunit beta (COPβ) (Zhu, F. et al. (2011) Ingested RNA interference for managing the populations of the Colorado potato beetle, Leptinotarsa decemlineata. Pest Manag Sci 67: 175-182). A screening platform using the red flour beetle Tribolium castaneum revealed that the proteasome is a prime target for RNAi-based pest control (Ulrich, J. et al. (2015) Large scale RNAi screen in Tribolium reveals novel target genes for pest control and the proteasome as prime target. BMC Genomics 16: 674). Other genes that have been targeted for down-regulation in RNAi-based insect pest control are reviewed in Zhang et al. 2013 (Zhang, H. et al. (2013) Feasibility, limitation and possible solutions of RNAi-based technology for insect pest control. Insect Science 20: 15-30).

### Strategies for Identifying Target Genes for RNAi-Based Pest Control

Strategies for identifying target genes for RNAi-based pest control include using known functional or homologous genes, searching sequenced genomes, sequencing of cDNA generated from RNA (RNA-seq), RNA-seq combined with digital gene expression tag profile (DGE-tag) and RNAi target sequencing (RIT-seq).

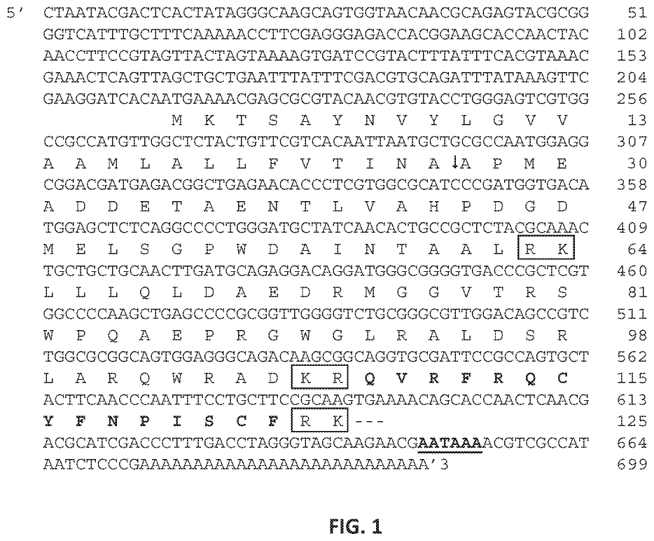

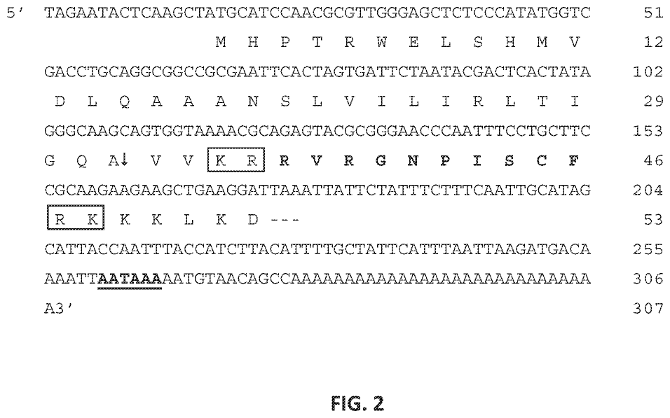

TABLE 3
Representative RNAi targets

| RNAi or RNA | Target Accession | | |
|---|---|---|---|
| targets | number(s)/Target reference | Effect | Target species |
| Rack-1, C002 | CN763138 (C002), U96491.1 | Rack-1, receptor for activated C | Myzus persicae |
| (MpC002) | (Rack-1)/Pitino M, Coleman A D, | kinase 1 regulating cell proliferation | |
| | Maffei M E, Ridout C J, | and growth; MpC002, expressed in | |
| | Hogenhout S A (2011) | salivary glands & responsible for | |
| | Silencing of Aphid Genes by | aphid interaction with plant host. | |
| | dsRNA Feeding from Plants. | Silencing either gene reduced | |
| | PLoS ONE 6(10): e25709. | fecundity | |
| Prothoracicotropic | AY286543.1 | Secreted by neurosecretory cells in | H. armigera |
| Hormone (PTTH) | AY780527.1/Choudhary, M. | larval brain, PTTH acts on the | |
| | and Sahi, S. (2011) In silico | prothoracic glands. PTTH and | |
| | designing of insecticidal small | ecydysone trigger every molt: larva- | |
| | interfering RNA (siRNA) for | to-larva as well as pupa-to-adult. | |
| | Helicoverpa armigera control. | | |
| | Indian Journal of Experimental | | |
| | Biology, 49(6): 469-474 | | |
| Molt-regulating | AF337637.3 | Regulates the expression of tissue- | H. armigera |
| transcription | FJ009448.1/Choudhary et al. | specific genes involved in insect | |
| factors3 (HR3) | 2011 | molting and metamorphosis | |
| Eclosion hormone | AY822476.1/Choudhary et al. | Triggers ecdysis behavior at the end | H. armigera |
| precursor (EH) | 2011 | of each molt, EH gene may possess | |
| | | other biological functions in post- | |
| | | embryonic development, is expressed | |
| | | through all the developmental stages | |
| Glutathione-S- | EF033109/Mao, Y. B. et al. | Catabolism of gossypol, a toxin of | H. armigera/zea |
| transferase gene | (2007) Silencing a cotton | cotton | |
| (GST1 dsRNA) | bollworm P450 | | |
| | monooxygenase gene by plant- | | |
| | mediated RNAi impairs larval | | |
| | tolerance of gossypol. Nature | | |
| | Biotechnology 25: 1307-1313 | | |
| P450 | DQ986461/Mao et al. 2007 | Furanocoumarin inducible catabolism | H. armigera/zea |
| monoxygenase | | of gossypol, a toxin of cotton | |
| (CYP6AE14 | | | |
| dsRNA) | | | |
| Allatostatin-C-type- | Spofr-AS | Synthesis of juvenile hormone which | S. frugiperda |
| sequence (AS-C- | See FIG. 1 of present | controls growth rates, molting and | |
| dsRNA) | application | pupa eclosion. JH likely possess | |
| | (FIG. 4 of Abdel-latief et al. | other biological functions in post- | |
| | 2003. Molecular | embryonic development, is expressed | |
| | characterization | through all the developmental stages | |
| | of cDNAs from the fall | | |
| | armyworm Spodoptera | | |
| | frugiperda encoding | | |
| | Manduca sexta allatotropin and | | |
| | allatostatin preprohormone | | |
| | peptides. Insect | | |
| | Biochemistry and Molecular | | |
| | Biology 33: 467-476) | | |
| Allatotropin 2 | See FIG. 2 of present | Synthesis of juvenile hormone (JH) | S. frugiperda |
| sequence (AT 2- | application | which controls growth rates, molting | |
| dsRNA) | (FIG. 2 of Abdel-latief et al. | and pupa eclosion. JH likely possess | |
| | 2004. Characterization of a | other biological functions in post- | |
| | novel peptide with allatotropic | embryonic development, is expressed | |
| | activity in the fall armyworm | through all the developmental stages | |
| | Spodoptera frugiperda. | | |
| | Regulatory Peptides 122: 69-78) | | |
| brahma, mi-2, iswi- | Brahma. KR152260 | Targets chromatin-remodeling | Nezara viridula |
| 1, iswi-2, chd1 | (Diabrotica virgifera virgifera) | ATPase transcripts that lead to | and Euchistus |
| | & KT369801 (Euschistus | reduced fecundity via decreased | heros |
| | heros); mi-2: KT364639 | oviposition and increased egg | |
| | (Diabrotica virgifera virgifera) | mortality. | |
| | & KT369802 (Euschistus | | |
| | heros); iswi-1: KT364640 | | |
| | (Diabrotica virgifera virgifera) | | |
| | & KT369803 (Euschistus | | |
| | heros); iswi-2: | | |
| | KT364641 (Diabrotica | | |
| | virgifera virgifera) & | | |
| | KT369804 (Euschistus heros); | | |
| | chd-1: KT364642 (Diabrotica | | |
| | virgifera virgifera) & | | |
| | KT369805 (Euschistus heros)/ | | |
| | Fishilevich et al. (2016) Use of | | |
| | chromatin remodeling ATPases | | |
| | as RNAi targets for parental | | |
| | control of western corn | | |
| | rootworm (Diabrotica virgifera | | |
| | virgifera) and Neotropical | | |
| | brown stink bug (Euschistus | | |
| | heros). Insect biochemistry and | | |
| | molecular biology, 71: 58-71 | | |
| 3-hydroxy-3- | GU584103/Tian et al. (2015) | catalyze a rate-limiting enzymatic | H. armigera |
| methylglutaryl | Transgenic cotton plants | reaction in the mevalonate pathway | |
| coenzyme A | expressing double-stranded | of juvenile hormone (JH) synthesis | |
| reductase (HMGR) | RNAs target HMG-CoA | | |
| | reductase (HMGR) gene | | |
| | inhibits the growth, | | |
| | development and survival of | | |
| | Cotton Bollworms. | | |
| | International Journal of | | |
| | Biological Sciences 11: 1296-1305 | | |
| Vacuolar-type H⁺- | pIC17504/Baum et al. 2007. | Acidification of a wide array of | Diabrotica |
| ATPase (V-ATPase | Control of coleopteran insect | intracellular organelles and pump | undecimpunctata |
| A subunit 2; V- | pests through RNA | protons across the plasma membranes | howardii/ |
| ATPase E) | interference. Nature | of numerous cell types. | Diabrotica |
| | biotechnology 25: 1322-1326. | | virgifera virgifera/ |
| | | | Leptinotarsa |
| | | | decemlineata |
| Ds10 | GH999144 (Ds10), GH997930 | chymotrypsin-like serine proteinase | Crambidae |
| Ds28 | (Ds28)/Wang, Y.; Zhang, H.; | C3 (Ds10) | Ostrinia furnalalis |
| | Li, H.; Miao, X. Second- | unknown gene function (Ds28) | |
| | generation sequencing supply | Larval mortality increased | |
| | an effective way to screen | | |
| | RNAi targets in large scale for | | |
| | potential application in pest | | |
| | insect control. PLoS ONE | | |
| | 2011, 6, e18644 | | |
| 8 genes: chitinase7, | JF915770 (arf2) | Larval mortality increased | Noctuidae |
| PGCP chitinase1, | JQ653045 (arf1) | | Spodoptera exigua |
| ATPase, tubulin1, | JQ653042 (tubulin1) | | |
| arf2, tubulin2 and | JQ653043 (tubulin2) | | |
| arf1 | JQ653040 (chitinase1) | | |
| | JQ653039 (chitinase7) | | |
| | JQ653044 (PGCP) | | |
| | JQ653046 (ATPase) | | |
| | Li, H.; Jiang, W.; Zhang, Z.; | | |
| | Xing, Y.; Li, F. Transcriptome | | |
| | analysis and screening for | | |
| | potential target genes for | | |
| | RNAi-mediated pest control of | | |
| | the beet armyworm, | | |
| | Spodoptera exigua. PLoS ONE | | |
| | 2013, 8, e65931 | | |
| cullin-1 | KP236737/Wang, J. D.; Gu, L. Q.; | Cullin-1 is involved in proteolysis | Tortricidae |
| | Ireland, S.; Garczynski, S. F.; | and is a component of 28S | Cydia pomonella |
| | Knipple, D. C. Phenotypic | proteasome | |
| | screen for RNAi effects in the | Larval length reduced | |
| | codling moth Cydia pomonella. | | |
| | Gene 2015, 572, 184-190 | | |
| acetylcholine | AY142325, AF369793, | key enzyme in the insect central | Noctuidae |
| esterase AChE | AY686704, | nervous system | Helicoverpa |
| | AY686705 | Mortality increased, growth | armigera |
| | Kumar, M.; Gupta, G. P.; | inhibition of larvae, reduction in the | |
| | Rajam, M. V. Silencing of | pupal weight, malformation and | |
| | acetylcholinesterase gene of | drastically reduced fecundity | |
| | Helicoverpa armigera by | | |
| | siRNA affects larval growth | | |
| | and its life cycle. J. Insect | | |
| | Physiol. 2009, 55, 273-278 | | |
| β1 integrin | ACS66819/Mohamed, A. A. M.; | Mediates signal transduction through | Plutellidae |
| | Kim, Y. A target- | the cell membrane | Plutella xylostella |
| | specific feeding toxicity of β1 | Larval mortality increased | |
| | integrin dsRNA against | | |
| | diamondback moth, Plutella | | |
| | xylostella. Arch. Insect | | |
| | Biochem. Physiol. 2011, 78, | | |
| | 216-230 | | |
| iron-sulfur protein | EU815629/Gong, L. A.; Yang, X. Q.; | Part of cytochrome bc1 complex, a | Plutellidae |
| | Zhang, B. L.; Zhong, G. H.; | central segment of the energy- | Plutella xylostella |
| | Hu, M. Y. Silencing of | conserving, electron transfer chain of | |
| | Rieske iron-sulfur protein using | the mitochondria. This enzyme | |
| | chemically synthesised siRNA | complex catalyses electron | |
| | as a potential biopesticide | transfer from ubiquinol to | |
| | against Plutella xylostella. Pest | cytochrome c with concomitant | |
| | Manag. Sci. 2011, 67, 514-520 | translocation of protons across the | |
| | | membrane to generate a | |
| | | proton electrochemical gradient | |
| | | required for ATP synthesis by | |
| | | ATP synthase | |
| | | Larval mortality increased | |
| Acetylcholine | AY061975 and AY970293 | key enzyme in the insect central | Plutellidae |
| esterase AChE2 | Gong, L.; Chen, Y.; Hu, Z.; | nervous system | Plutella xylostella |
| | Hu, M. Y. Testing insecticidal | Larval mortality increased | |
| | activity of novel chemically | | |
| | synthesized siRNA against | | |
| | Plutella xylostella under | | |
| | laboratory and field conditions. | | |
| | PLoS ONE 2013, 8, e62990 | | |
| aminopeptidaseN | KF290773/Kola, V. S. R.; | receptor in Cry toxin-induced | Pyralidae |
| | Renuka, P. Padmakumari, A. P.; | pathogenesis in insects | Scirpophaga |
| | Mangrauthia, S. K.; | Larval mortality increased, larval | incertulas |
| | Balachandran, S. M.; Babu, V. R.; | weight reduced | |
| | Madhav, M. S. | | |
| | Silencing of CYP6 and APN | | |
| | genes affects the growth and | | |
| | development of rice yellow | | |
| | stem borer, Scirpophaga | | |
| | incertulas. Front. Physiol. 2016 | | |
| vATPase | NM_169073, X67131, | a membrane-bound protein that | Sphingidae |
| | XM_965528 and | acts as a proton pump to establish the | Manduca sexta |
| | XM_001946489 | pH gradient within the gut | |
| | Whyard, S.; Singh, A. D.; | lumen of many insects | |
| | Wong, S. Ingested | Larval mortality increased | |
| | double-stranded RNAs can act | | |
| | as species-specific insecticides. | | |
| | Insect Biochem. Mol. Biol. | | |
| | 2009, 39, 824-832 | | |
| arginine kinase | EF600057/Qi, X. L.; Su, X. F.; | may be involved in hormone | Noctuidae |
| | Lu, G. Q.; Liu, C. X.; Liang, G. M.; | signalling pathway and larval | Helicoverpa |
| | Cheng, H. M. The effect | development | armigera |
| | of silencing arginine kinase by | Mortality increased | |
| | RNAi on the larval | | |
| | development of Helicoverpa | | |
| | armigera. Bull. Entomol. Res. | | |
| | 2015, 105, 555-565 | | |
| cytochrome P450 | AY950636/Zhang, X.; Liu, X.; | involved in regulating the | Noctuidae |
| CYP6B6 | Ma, J.; Zhao, J. Silencing of | titers of endogenous compounds such | Helicoverpa |
| | cytochrome P450 CYP6B6 | as hormones, fatty acids | armigera |
| | gene of cotton bollworm | and steroids | |
| | (Helicoverpa armigera) by | Mortality increased | |
| | RNAi. Bull. Entomol. Res. | | |
| | 2013, 103, 584-591 | | |
| chitin synthase A | DQ062153/Tian, H.; Peng, H.; | key enzyme for cuticle, trachea, and | Noctuidae |
| | Yao, Q.; Chen, H.; Xie, Q.; | midgut development; chitin synthase | Spodoptera exigua |
| | Tang, B.; Zhang, W. | A genes are specifically expressed | |
| | Developmental control of a | in ectodermal cells, including epidermal | |
| | Lepidopteran pest Spodoptera | and tracheal cells | |
| | exigua by ingestion of bacteria | Larval mortality increased | |
| | expressing dsRNA of a non- | | |
| | midgut gene. PLoS ONE 2009, | | |
| | 4, e6225 | | |
| cytochrome P450 | DQ986461/Mao, Y.-B.; Tao, X.-Y.; | Larval growth decreased, rate of leaf | Noctuidae |
| CYP6AE14 | Xue, X.-Y.; Wang, L.-J.; | consumption reduced | Helicoverpa |
| | Chen, X.-Y. Cotton plants | | armigera |
| | expressing CYP6AE14 double- | | |
| | stranded RNA show enhanced | | |
| | resistance to bollworms. | | |
| | Transgenic Res. 2011, 20, 665-673 | | |
| chitinase | AY326455/Mamta; Reddy, K. R. K.; | Mortality increased | Noctuidae |
| | Rajam, M. V. Targeting | | Helicoverpa |
| | chitinase gene of Helicoverpa | | armigera |
| | armigera by host-induced RNA | | |
| | interference confers insect | | |
| | resistance in tobacco and | | |
| | tomato. Plant Mol. Biol. 2016, | | |
| | 90, 281-292 | | |

### Pheromone Formulations

The pheromone formulations used in the methods of the invention may be provided alone or may be included in a carrier and/or a dispenser. In one embodiment, the methods comprise applying one or more pheromones in dispensers located throughout the entire field plot. In another embodiment, the methods comprise applying one or more pheromone formulations comprising sprayable emulsion concentrate or sprayable microencapsulation formulations. In another embodiment, the methods comprise applying one or more pheromones in aerosol emitters.

A dispenser allows for release of the pheromone composition. Any suitable dispenser known in the art can be used. Examples of such dispensers include but are not limited to bubble caps comprising a reservoir with a permeable barrier through which pheromones are slowly released, pads, beads, tubes rods, spirals or balls composed of rubber, plastic, leather, cotton, cotton wool, wood or wood products that are impregnated with the pheromone composition. For example, polyvinyl chloride laminates, pellets, granules, ropes or spirals from which the pheromone composition evaporates, or rubber septa. An example of a dispenser is a sealed polyethylene tube containing the pheromone composition of the invention where a wire is fused inside the plastic so the dispenser can be attached by the wire to a tree or shrub. The dispenser may also comprise or include a trap. A killing agent may be incorporated into the trap, such as a sticky or insecticide-treated surface, a restricted exit, insecticide vapour or an electric grid.

The carrier may be an inert liquid or solid. Examples of solid carriers include but are not limited to fillers such as kaolin, bentonite, dolomite, calcium carbonate, talc, powdered magnesia, Fuller's earth, wax, gypsum, diatomaceous earth, rubber, plastic, silica and China clay. Examples of liquid carriers include but are not limited to water; alcohols, particularly ethanol, butanol or glycol, as well as their ethers or esters, particularly methylglycol acetate; ketones, particularly acetone, cyclohexanone, methylethyl ketone, methylisobutylketone, or isophorone; alkanes such as hexane, pentane, heptanes; aromatic hydrocarbons, particularly xylenes or alkyl naphthalenes; mineral or vegetable oils; aliphatic chlorinated hydrocarbons, particularly trichloroethane or methylene chloride; aromatic chlorinated hydrocarbons, particularly chlorobenzenes; water-soluble or strongly polar solvents such as dimethylformamide, dimethyl sulfoxide, or N-methylpyrrolidone; liquefied gases; or the like or a mixture thereof.

The pheromone formulations used in the methods of the invention may be formulated so as to provide slow release into the atmosphere, and/or so as to be protected from degradation following release. For example, the pheromone formulations may comprise carriers such as microcapsules, biodegradable flakes and paraffin wax-based matrices. In some instances the pheromone composition is provided by direct release from the carrier. For example, Min-U-Gel™, a highly absorptive Attapulgite clay, can be impregnated with a pheromone composition of the invention. In another example, the pheromone composition may be mixed in a carrier paste that can be applied to trees and other plants. Insecticides may be added to the paste. Baits or feeding stimulants can also be added to the carrier.

The pheromone formulations used in the methods of the invention may comprise other pheromones or attractants provided that the other compounds do not substantially interfere with the activity of the formulations.

Mating disruption formulations can include the following categories, depending upon dispenser type and application technique: (1) Reservoir, high rate systems that must be hand applied; (2) female equivalent, low rate sprayable systems; (3) female equivalent, low rate hand-applied systems; (3) microdispersible, low rate systems that are sprayable. Commercial mating disruption and attract and kill formulations for pink bollworm are summarized in Jenkins 2002 (Jenkins, J. W. Use of mating disruption in cotton in North and South America. Use of pheromones and other semiochemicals in integrated production, IOBC wprs Bulletin Vol. 25, 2002) and is herein incorporated in its entirety.

As described above, products made via the methods described herein are pheromones. Pheromones prepared according to the methods of the invention can be formulated for use as insect control compositions. The pheromone compositions can include a carrier, and/or be contained in a dispenser. The carrier can be, but is not limited to, an inert liquid or solid. Examples of solid carriers include but are not limited to fillers such as kaolin, bentonite, dolomite, calcium carbonate, talc, powdered magnesia, Fuller's earth, wax, gypsum, diatomaceous earth, rubber, plastic, China clay, mineral earths such as silicas, silica gels, silicates, attaclay, limestone, chalk, loess, clay, dolomite, calcium sulfate, magnesium sulfate, magnesium oxide, ground synthetic materials, fertilizers such as ammonium sulfate, ammonium phosphate, ammonium nitrate, thiourea and urea, products of vegetable origin such as cereal meals, tree bark meal, wood meal and nutshell meal, cellulose powders, attapulgites, montmorillonites, mica, vermiculites, synthetic silicas and synthetic calcium silicates, or compositions of these.

Examples of liquid carriers include, but are not limited to, water; alcohols, such as ethanol, butanol or glycol, as well as their ethers or esters, such as methylglycol acetate; ketones, such as acetone, cyclohexanone, methylethyl ketone, methylisobutylketone, or isophorone; alkanes such as hexane, pentane, or heptanes; aromatic hydrocarbons, such as xylenes or alkyl naphthalenes; mineral or vegetable oils; aliphatic chlorinated hydrocarbons, such as trichloroethane or methylene chloride; aromatic chlorinated hydrocarbons, such as chlorobenzenes; water-soluble or strongly polar solvents such as dimethylformamide, dimethyl sulfoxide, or N-methylpyrrolidone; liquefied gases; waxes, such as beeswax, lanolin, shellac wax, carnauba wax, fruit wax (such as bayberry or sugar cane wax) candelilla wax, other waxes such as microcrystalline, ozocerite, ceresin, or montan; salts such as monoethanolamine salt, sodium sulfate, potassium sulfate, sodium chloride, potassium chloride, sodium acetate, ammonium hydrogen sulfate, ammonium chloride, ammonium acetate, ammonium formate, ammonium oxalate, ammonium carbonate, ammonium hydrogen carbonate, ammonium thiosulfate, ammonium hydrogen diphosphate, ammonium dihydrogen monophosphate, ammonium sodium hydrogen phosphate, ammonium thiocyanate, ammonium sulfamate or ammonium carbamate and mixtures thereof. Baits or feeding stimulants can also be added to the carrier.

### Synergist

In some embodiments, the pheromone composition is combined with an active chemical agent such that a synergistic effect results. The synergistic effect obtained by the taught methods can be quantified according to Colby's formula (i.e. (E)=X+Y−(X*Y/100). See Colby, R. S., “Calculating Synergistic and Antagonistic Responses of Herbicide Combinations”, 1967 Weeds, vol. 15, pp. 20-22, incorporated herein by reference in its entirety. Thus, by “synergistic” is intended a component which, by virtue of its presence, increases the desired effect by more than an additive amount. The pheromone compositions and adjuvants of the present methods can synergistically increase the effectiveness of agricultural active compounds and also agricultural auxiliary compounds.

Thus, in some embodiments, a pheromone composition can be formulated with a synergist. The term, “synergist,” as used herein, refers to a substance that can be used with a pheromone for reducing the amount of the pheromone dose or enhancing the effectiveness of the pheromone for attracting at least one species of insect. The synergist may or may not be an independent attractant of an insect in the absence of a pheromone. In some embodiments, the synergist is a volatile phytochemical that attracts at least one species of Lepidoptera. The term, “phytochemical,” as used herein, means a compound occurring naturally in a plant species. In a particular embodiment, the synergist is selected from the group comprising β-caryophyllene, iso-caryophyllene, α-humulene, inalool, Z3-hexenol/yl acetate, β-farnesene, benzaldehyde, phenylacetaldehyde, and combinations thereof. The pheromone composition can contain the pheromone and the synergist in a mixed or otherwise combined form, or it may contain the pheromone and the synergist independently in a non-mixed form.

### Insecticide

The pheromone composition can include one or more insecticides. In one embodiment, the insecticides are chemical insecticides known to one skilled in the art. Examples of the chemical insecticides include one or more of pyrethoroid or organophosphorus insecticides, including but are not limited to, cyfluthrin, permethrin, cypermethrin, bifinthrin, fenvalerate, flucythrinate, azinphosmethyl, methyl parathion, buprofezin, pyriproxyfen, flonicamid, acetamiprid, dinotefuran, clothianidin, acephate, malathion, quinolphos, chloropyriphos, profenophos, bendiocarb, bifenthrin, chlorpyrifos, cyfluthrin, diazinon, pyrethrum, fenpropathrin, kinoprene, insecticidal soap or oil, neonicotinoids, diamides, avermectin and derivatives, spinosad and derivatives, azadirachtin, pyridalyl, and mixtures thereof.

In another embodiment, the insecticides are one or more biological insecticides known to one skilled in the art. Examples of the biological insecticides include, but are not limited to, azadirachtin (neem oil), toxins from natural pyrethrins, Bacillus thuringiencis and Beauveria bassiana, viruses (e.g., CYD-X™, CYD-X HP™, Germstar™, Madex HP™ and Spod-X™), peptides (Spear-T™, Spear-P™, and Spear-C™).

In another embodiment, the insecticides are insecticides that target the nerve and muscle. Examples include acetylcholinesterase (AChE) inhibitors, such as carbamates (e.g., methomyl and thiodicarb) and organophosphates (e.g., chlorpyrifos) GABA-gated chloride channel antagonists, such as cyclodiene organochlorines (e.g., endosulfan) and phenylpyrazoles (e.g., fipronil), sodium channel modulators, such as pyrethrins and pyrethroids (e.g., cypermethrin and k-cyhalothrin), nicotinic acetylcholine receptor (nAChR) agonists, such as neonicotinoids (e.g., acetamiprid, tiacloprid, thiamethoxam), nicotinic acetylcholine receptor (nAChR) allosteric modulators, such as spinosyns (e.g., spinose and spinetoram), chloride channel activators, such as avermectins and milbemycins (e.g., abamectin, emamectin benzoate), Nicotinic acetylcholine receptor (nAChR) blockers, such as bensultap and cartap, voltage dependent sodium channel blockers, such as indoxacarb and metaflumizone, ryanodine receptor modulator, such as diamides (e.g. dhlorantraniliprole and flubendiamide). In another embodiment, the insecticides are insecticides that target respiration. Examples include chemicals that uncouple oxidative phosphorylation via disruption of the proton gradient, such as chlorfenapyr, and mitochondrial complex I electron transport inhibitors.

In another embodiment, the insecticides are insecticides that target midgut. Examples include microbial disruptors of insect midgut membranes, such as Bacillus thuringiensis and Bacillus sphaericus.

In another embodiment, the insecticides are insecticides that target growth and development. Examples include juvenile hormone mimics, such as juvenile hormone analogues (e.g. fenoxycarb), inhibitors of chitin biosynthesis, Type 0, such as benzoylureas (e.g., flufenoxuron, lufenuron, and novaluron), and ecdysone receptor agonists, such as diacylhydrazines (e.g., methoxyfenozide and tebufenozide)

### Stabilizer

According to another embodiment of the disclosure, the pheromone composition may include one or more additives that enhance the stability of the composition. Examples of additives include, but are not limited to, fatty acids and vegetable oils, such as for example olive oil, soybean oil, corn oil, safflower oil, canola oil, and combinations thereof.

### Filler

According to another embodiment of the disclosure, the pheromone composition may include one or more fillers. Examples of fillers include, but are not limited to, one or more mineral clays (e.g., attapulgite). In some embodiments, the attractant-composition may include one or more organic thickeners. Examples of such thickeners include, but are not limited to, methyl cellulose, ethyl cellulose, and any combinations thereof.

### Solvent

According to another embodiment, the pheromone compositions of the present disclosure can include one or more solvents. Compositions containing solvents are desirable when a user is to employ liquid compositions which may be applied by brushing, dipping, rolling, spraying, or otherwise applying the liquid compositions to substrates on which the user wishes to provide a pheromone coating (e.g., a lure). In some embodiments, the solvent(s) to be used is/are selected so as to solubilize, or substantially solubilize, the one or more ingredients of the pheromone composition. Examples of solvents include, but are not limited to, water, aqueous solvent (e.g., mixture of water and ethanol), ethanol, methanol, chlorinated hydrocarbons, petroleum solvents, turpentine, xylene, and any combinations thereof.

In some embodiments, the pheromone compositions of the present disclosure comprise organic solvents. Organic solvents are used mainly in the formulation of emulsifiable concentrates, ULV formulations, and to a lesser extent granular formulations. Sometimes mixtures of solvents are used. In some embodiments, the present disclosure teaches the use of solvents including aliphatic paraffinic oils such as kerosene or refined paraffins. In other embodiments, the present disclosure teaches the use of aromatic solvents such as xylene and higher molecular weight fractions of C9 and C10 aromatic solvents. In some embodiments, chlorinated hydrocarbons are useful as co-solvents to prevent crystallization when the formulation is emulsified into water. Alcohols are sometimes used as co-solvents to increase solvent power.

### Solubilizing Agent

In some embodiments, the pheromone compositions of the present disclosure comprise solubilizing agents. A solubilizing agent is a surfactant, which will form micelles in water at concentrations above the critical micelle concentration. The micelles are then able to dissolve or solubilize water-insoluble materials inside the hydrophobic part of the micelle. The types of surfactants usually used for solubilization are non-ionics: sorbitan monooleates; sorbitan monooleate ethoxylates; and methyl oleate esters.

### Binder

According to another embodiment of the disclosure, the pheromone composition may include one or more binders. Binders can be used to promote association of the pheromone composition with the surface of the material on which said composition is coated. In some embodiments, the binder can be used to promote association of another additive (e.g., insecticide, insect growth regulators, and the like) to the pheromone composition and/or the surface of a material. For example, a binder can include a synthetic or natural resin typically used in paints and coatings. These may be modified to cause the coated surface to be friable enough to allow insects to bite off and ingest the components of the composition (e.g., insecticide, insect growth regulators, and the like), while still maintaining the structural integrity of the coating.

Non-limiting examples of binders include polyvinylpyrrolidone, polyvinyl alcohol, partially hydrolyzed polyvinyl acetate, carboxymethylcellulose, starch, vinylpyrrolidone/vinyl acetate copolymers and polyvinyl acetate, or compositions of these; lubricants such as magnesium stearate, sodium stearate, talc or polyethylene glycol, or compositions of these; antifoams such as silicone emulsions, long-chain alcohols, phosphoric esters, acetylene diols, fatty acids or organofluorine compounds, and complexing agents such as: salts of ethylenediaminetetraacetic acid (EDTA), salts of trinitrilotriacetic acid or salts of polyphosphoric acids, or compositions of these.

In some embodiments, the binder also acts a filler and/or a thickener. Examples of such binders include, but are not limited to, one or more of shellac, acrylics, epoxies, alkyds, polyurethanes, linseed oil, tung oil, and any combinations thereof.

### Surface-Active Agents

In some embodiments, the pheromone compositions comprise surface-active agents. In some embodiments, the surface-active agents are added to liquid agricultural compositions. In other embodiments, the surface-active agents are added to solid formulations, especially those designed to be diluted with a carrier before application. Thus, in some embodiments, the pheromone compositions comprise surfactants. Surfactants are sometimes used, either alone or with other additives, such as mineral or vegetable oils as adjuvants to spray-tank mixes to improve the biological performance of the pheromone on the target. The surface-active agents can be anionic, cationic, or nonionic in character, and can be employed as emulsifying agents, wetting agents, suspending agents, or for other purposes. In some embodiments, the surfactants are non-ionics such as: alky ethoxylates, linear aliphatic alcohol ethoxylates, and aliphatic amine ethoxylates. Surfactants conventionally used in the art of formulation and which may also be used in the present formulations are described, in McCutcheon's Detergents and Emulsifiers Annual, MC Publishing Corp., Ridgewood, N.J., 1998, and in Encyclopedia of Surfactants, Vol. I-III, Chemical Publishing Co., New York, 1980-81. In some embodiments, the present disclosure teaches the use of surfactants including alkali metal, alkaline earth metal or ammonium salts of aromatic sulfonic acids, for example, ligno-, phenol-, naphthalene- and dibutylnaphthalenesulfonic acid, and of fatty acids of arylsulfonates, of alkyl ethers, of lauryl ethers, of fatty alcohol sulfates and of fatty alcohol glycol ether sulfates, condensates of sulfonated naphthalene and its derivatives with formaldehyde, condensates of naphthalene or of the naphthalenesulfonic acids with phenol and formaldehyde, condensates of phenol or phenolsulfonic acid with formaldehyde, condensates of phenol with formaldehyde and sodium sulfite, polyoxyethylene octylphenyl ether, ethoxylated isooctyl-, octyl- or nonylphenol, tributylphenyl polyglycol ether, alkylaryl polyether alcohols, isotridecyl alcohol, ethoxylated castor oil, ethoxylated triarylphenols, salts of phosphated triarylphenolethoxylates, lauryl alcohol polyglycol ether acetate, sorbitol esters, lignin-sulfite waste liquors or methylcellulose, or compositions of these.

In some embodiments, the present disclosure teaches other suitable surface-active agents, including salts of alkyl sulfates, such as diethanolammonium lauryl sulfate; alkylarylsulfonate salts, such as calcium dodecylbenzenesulfonate; alkylphenol-alkylene oxide addition products, such as nonylphenol-C18 ethoxylate; alcohol-alkylene oxide addition products, such as tridecyl alcohol-C16 ethoxylate; soaps, such as sodium stearate; alkylnaphthalene-sulfonate salts, such as sodium dibutyl-naphthalenesulfonate; dialkyl esters of sulfosuccinate salts, such as sodium di(2-ethylhexyl)sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; salts of mono and dialkyl phosphate esters; vegetable oils such as soybean oil, rapeseed/canola oil, olive oil, castor oil, sunflower seed oil, coconut oil, corn oil, cottonseed oil, linseed oil, palm oil, peanut oil, safflower oil, sesame oil, tung oil and the like; and esters of the above vegetable oils, particularly methyl esters.

### Wetting Agents

In some embodiments, the pheromone compositions comprise wetting agents. A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank or other vessel to reduce the wetting time of wettable powders and to improve the penetration of water into water-dispersible granules. In some embodiments, examples of wetting agents used in the pheromone compositions of the present disclosure, including wettable powders, suspension concentrates, and water-dispersible granule formulations are: sodium lauryl sulphate; sodium dioctyl sulphosuccinate; alkyl phenol ethoxylates; and aliphatic alcohol ethoxylates.

### Dispersing Agent

In some embodiments, the pheromone compositions of the present disclosure comprise dispersing agents. A dispersing agent is a substance which adsorbs onto the surface of particles and helps to preserve the state of dispersion of the particles and prevents them from reaggregating. In some embodiments, dispersing agents are added to pheromone compositions of the present disclosure to facilitate dispersion and suspension during manufacture, and to ensure the particles redisperse into water in a spray tank. In some embodiments, dispersing agents are used in wettable powders, suspension concentrates, and water-dispersible granules. Surfactants that are used as dispersing agents have the ability to adsorb strongly onto a particle surface and provide a charged or steric barrier to re-aggregation of particles. In some embodiments, the most commonly used surfactants are anionic, non-ionic, or mixtures of the two types. In some embodiments, for wettable powder formulations, the most common dispersing agents are sodium lignosulphonates. In some embodiments, suspension concentrates provide very good adsorption and stabilization using polyelectrolytes, such as sodium naphthalene sulphonate formaldehyde condensates. In some embodiments, tristyrylphenol ethoxylated phosphate esters are also used. In some embodiments, such as alkylarylethylene oxide condensates and EO-PO block copolymers are sometimes combined with anionics as dispersing agents for suspension concentrates.

### Polymeric Surfactant

In some embodiments, the pheromone compositions of the present disclosure comprise polymeric surfactants. In some embodiments, the polymeric surfactants have very long hydrophobic ‘backbones’ and a large number of ethylene oxide chains forming the ‘teeth’ of a ‘comb’ surfactant. In some embodiments, these high molecular weight polymers can give very good long-term stability to suspension concentrates, because the hydrophobic backbones have many anchoring points onto the particle surfaces. In some embodiments, examples of dispersing agents used in pheromone compositions of the present disclosure are: sodium lignosulphonates; sodium naphthalene sulphonate formaldehyde condensates; tristyrylphenol ethoxylate phosphate esters; aliphatic alcohol ethoxylates; alky ethoxylates; EO-PO block copolymers; and graft copolymers.

### Emulsifying Agent

In some embodiments, the pheromone compositions of the present disclosure comprise emulsifying agents. An emulsifying agent is a substance, which stabilizes a suspension of droplets of one liquid phase in another liquid phase. Without the emulsifying agent the two liquids would separate into two immiscible liquid phases. In some embodiments, the most commonly used emulsifier blends include alkylphenol or aliphatic alcohol with 12 or more ethylene oxide units and the oil-soluble calcium salt of dodecylbenzene sulphonic acid. A range of hydrophile-lipophile balance (“HLB”) values from 8 to 18 will normally provide good stable emulsions. In some embodiments, emulsion stability can sometimes be improved by the addition of a small amount of an EO-PO block copolymer surfactant.

### Gelling Agent

In some embodiments, the pheromone compositions comprise gelling agents. Thickeners or gelling agents are used mainly in the formulation of suspension concentrates, emulsions, and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. In some embodiments, the pheromone compositions comprise one or more thickeners including, but not limited to: montmorillonite, e.g. bentonite; magnesium aluminum silicate; and attapulgite. In some embodiments, the present disclosure teaches the use of polysaccharides as thickening agents. The types of polysaccharides most commonly used are natural extracts of seeds and seaweeds or synthetic derivatives of cellulose. Some embodiments utilize xanthan and some embodiments utilize cellulose. In some embodiments, the present disclosure teaches the use of thickening agents including, but are not limited to: guar gum; locust bean gum; carrageenam; alginates; methyl cellulose; sodium carboxymethyl cellulose (SCMC); hydroxyethyl cellulose (HEC). In some embodiments, the present disclosure teaches the use of other types of anti-settling agents such as modified starches, polyacrylates, polyvinyl alcohol, and polyethylene oxide. Another good anti-settling agent is xanthan gum.

### Anti-Foam Agent

In some embodiments, the presence of surfactants, which lower interfacial tension, can cause water-based formulations to foam during mixing operations in production and in application through a spray tank. Thus, in some embodiments, in order to reduce the tendency to foam, anti-foam agents are often added either during the production stage or before filling into bottles/spray tanks. Generally, there are two types of anti-foam agents, namely silicones and nonsilicones. Silicones are usually aqueous emulsions of dimethyl polysiloxane, while the nonsilicone anti-foam agents are water-insoluble oils, such as octanol and nonanol, or silica. In both cases, the function of the anti-foam agent is to displace the surfactant from the air-water interface.

### Preservative

In some embodiments, the pheromone compositions comprise a preservative.

### Additional Active Agent

According to another embodiment of the disclosure, the pheromone composition may include one or more insect feeding stimulants. Examples of insect feeding stimulants include, but are not limited to, crude cottonseed oil, fatty acid esters of phytol, fatty acid esters of geranyl geraniol, fatty acid esters of other plant alcohols, plant extracts, and combinations thereof. According to another embodiment of the disclosure, the pheromone composition may include one or more insect growth regulators (“IGRs”). IGRs may be used to alter the growth of the insect and produce deformed insects. Examples of insect growth regulators include, for example, dimilin.

According to another embodiment of the disclosure, the attractant-composition may include one or more insect sterilants that sterilize the trapped insects or otherwise block their reproductive capacity, thereby reducing the population in the following generation. In some situations allowing the sterilized insects to survive and compete with non-trapped insects for mates is more effective than killing them outright.

### Sprayable Compositions

In some embodiments, the pheromone compositions disclosed herein can be formulated as a sprayable composition (i.e., a sprayable pheromone composition). An aqueous solvent can be used in the sprayable composition, e.g., water or a mixture of water and an alcohol, glycol, ketone, or other water-miscible solvent. In some embodiments, the water content of such mixture is at least about 10%, at least about 20%, at least about 30%, at least about 40%, 50%, at least about 60%, at least about 70%, at least about 80%, or at least about 90%. In some embodiments, the sprayable composition is concentrate, i.e. a concentrated suspension of the pheromone, and other additives (e.g., a waxy substance, a stabilizer, and the like) in the aqueous solvent, and can be diluted to the final use concentration by addition of solvent (e.g., water).

In some embodiments, a waxy substance can be used as a carrier for the pheromone and its positional isomer in the sprayable composition. The waxy substance can be, e.g., a biodegradable wax, such as bees wax, carnauba wax and the like, candelilla wax (hydrocarbon wax), montan wax, shellac and similar waxes, saturated or unsaturated fatty acids, such as lauric, palmitic, oleic or stearic acid, fatty acid amides and esters, hydroxylic fatty acid esters, such as hydroxyethyl or hydroxypropyl fatty acid esters, fatty alcohols, and low molecular weight polyesters such as polyalkylene succinates.

In some embodiments, a stabilizer can be used with the sprayable pheromone compositions. The stabilizer can be used to regulate the particle size of concentrate and/or to allow the preparation of a stable suspension of the pheromone composition. In some embodiments, the stabilizer is selected from hydroxylic and/or ethoxylated polymers. Examples include ethylene oxide and propylene oxide copolymer, polyalcohols, including starch, maltodextrin and other soluble carbohydrates or their ethers or esters, cellulose ethers, gelatin, polyacrylic acid and salts and partial esters thereof and the like. In other embodiments, the stabilizer can include polyvinyl alcohols and copolymers thereof, such as partly hydrolyzed polyvinyl acetate. The stabilizer may be used at a level sufficient to regulate particle size and/or to prepare a stable suspension, e.g., between 0.1% and 15% of the aqueous solution.

In some embodiments, a binder can be used with the sprayable pheromone compositions. In some embodiments, the binder can act to further stabilize the dispersion and/or improve the adhesion of the sprayed dispersion to the target locus (e.g., trap, lure, plant, and the like). The binder can be polysaccharide, such as an alginate, cellulose derivative (acetate, alkyl, carboxymethyl, hydroxyalkyl), starch or starch derivative, dextrin, gum (arabic, guar, locust bean, tragacanth, carrageenan, and the like), sucrose, and the like. The binder can also be a non-carbohydrate, water-soluble polymer such as polyvinyl pyrrolidone, or an acidic polymer such as polyacrylic acid or polymethacrylic acid, in acid and/or salt form, or mixtures of such polymers.

### Microencapsulated Pheromones

In some embodiments, the pheromone compositions disclosed herein can be formulated as a microencapsulated pheromone, such as disclosed in Ill'lchev, A L et al., J. Econ. Entomol. 2006; 99(6):2048-54; and Stelinki, L L et al., J. Econ. Entomol. 2007; 100(4):1360-9. Microencapsulated pheromones (MECs) are small droplets of pheromone enclosed within polymer capsules. The capsules control the release rate of the pheromone into the surrounding environment, and are small enough to be applied in the same method as used to spray insecticides. The effective field longevity of the microencapsulated pheromone formulations can range from a few days to slightly more than a week, depending on inter alia climatic conditions, capsule size and chemical properties.

### Slow-Release Formulation

Pheromone compositions can be formulated so as to provide slow release into the atmosphere, and/or so as to be protected from degradation following release. For example, the pheromone compositions can be included in carriers such as microcapsules, biodegradable flakes and paraffin wax-based matrices. Alternatively, the pheromone composition can be formulated as a slow release sprayable.

In certain embodiments, the pheromone composition may include one or more polymeric agents known to one skilled in the art. The polymeric agents may control the rate of release of the composition to the environment. In some embodiments, the polymeric attractant-composition is impervious to environmental conditions. The polymeric agent may also be a sustained-release agent that enables the composition to be released to the environment in a sustained manner. Examples of polymeric agents include, but are not limited to, celluloses, proteins such as casein, fluorocarbon-based polymers, hydrogenated rosins, lignins, melamine, polyurethanes, vinyl polymers such as polyvinyl acetate (PVAC), polycarbonates, polyvinylidene dinitrile, polyamides, polyvinyl alcohol (PVA), polyamide-aldehyde, polyvinyl aldehyde, polyesters, polyvinyl chloride (PVC), polyethylenes, polystyrenes, polyvinylidene, silicones, and combinations thereof. Examples of celluloses include, but are not limited to, methylcellulose, ethyl cellulose, cellulose acetate, cellulose acetate-butyrate, cellulose acetate-propionate, cellulose propionate, and combinations thereof.

Other agents which can be used in slow-release or sustained-release formulations include fatty acid esters (such as a sebacate, laurate, palmitate, stearate or arachidate ester) or a fatty alcohols (such as undecanol, dodecanol, tridecanol, tridecenol, tetradecanol, tetradecenol, tetradecadienol, pentadecanol, pentadecenol, hexadecanol, hexadecenol, hexadecadienol, octadecenol and octadecadienol).

Pheromones prepared according to the methods of the invention, as well as compositions containing the pheromones, can be used to control the behavior and/or growth of insects in various environments. The pheromones can be used, for example, to attract or repel male or female insects to or from a particular target area. The pheromones can be used to attract insects away from vulnerable crop areas. The pheromones can also be used example to attract insects as part of a strategy for insect monitoring, mass trapping, lure/attract-and-kill or mating disruption.

### Lures

The pheromone compositions of the present disclosure may be coated on or sprayed on a lure, or the lure may be otherwise impregnated with a pheromone composition.

### Traps

The pheromone compositions of the disclosure may be used in traps, such as those commonly used to attract any insect species, e.g., insects of the order Lepidoptera. Such traps are well known to one skilled in the art, and are commonly used in many states and countries in insect eradication programs. In one embodiment, the trap includes one or more septa, containers, or storage receptacles for holding the pheromone composition. Thus, in some embodiments, the present disclosure provides a trap loaded with at least one pheromone composition. Thus, the pheromone compositions of the present disclosure can be used in traps for example to attract insects as part of a strategy for insect monitoring, mass trapping, mating disruption, or lure/attract and kill for example by incorporating a toxic substance into the trap to kill insects caught. Mass trapping involves placing a high density of traps in a crop to be protected so that a high proportion of the insects are removed before the crop is damaged. Lure/attract-and-kill techniques are similar except once the insect is attracted to a lure, it is subjected to a killing agent. Where the killing agent is an insecticide, a dispenser can also contain a bait or feeding stimulant that will entice the insects to ingest an effective amount of an insecticide. The insecticide may be an insecticide known to one skilled in the art. The insecticide may be mixed with the attractant-composition or may be separately present in a trap. Mixtures may perform the dual function of attracting and killing the insect.

Such traps may take any suitable form, and killing traps need not necessarily incorporate toxic substances, the insects being optionally killed by other means, such as drowning or electrocution. Alternatively, the traps can contaminate the insect with a fungus or virus that kills the insect later. Even where the insects are not killed, the trap can serve to remove the male insects from the locale of the female insects, to prevent breeding.

It will be appreciated by a person skilled in the art that a variety of different traps are possible. Suitable examples of such traps include water traps, sticky traps, and one-way traps. Sticky traps come in many varieties. One example of a sticky trap is of cardboard construction, triangular or wedge-shaped in cross-section, where the interior surfaces are coated with a non-drying sticky substance. The insects contact the sticky surface and are caught. Water traps include pans of water and detergent that are used to trap insects. The detergent destroys the surface tension of the water, causing insects that are attracted to the pan, to drown in the water. One-way traps allow an insect to enter the trap but prevent it from exiting. The traps of the disclosure can be colored brightly, to provide additional attraction for the insects.

In some embodiments, the pheromone traps containing the composition may be combined with other kinds of trapping mechanisms. For example, in addition to the pheromone composition, the trap may include one or more florescent lights, one or more sticky substrates and/or one or more colored surfaces for attracting moths. In other embodiments, the pheromone trap containing the composition may not have other kinds of trapping mechanisms.

The trap may be set at any time of the year in a field. Those of skill in the art can readily determine an appropriate amount of the compositions to use in a particular trap, and can also determine an appropriate density of traps/acre of crop field to be protected.

The trap can be positioned in an area infested (or potentially infested) with insects. Generally, the trap is placed on or close to a tree or plant. The aroma of the pheromone attracts the insects to the trap. The insects can then be caught, immobilized and/or killed within the trap, for example, by the killing agent present in the trap.

Traps may also be placed within an orchard to overwhelm the pheromones emitted by the females, so that the males simply cannot locate the females. In this respect, a trap need be nothing more than a simple apparatus, for example, a protected wickable to dispense pheromone. The traps of the present disclosure may be provided in made-up form, where the compound of the disclosure has already been applied. In such an instance, depending on the half-life of the compound, the compound may be exposed, or may be sealed in conventional manner, such as is standard with other aromatic dispensers, the seal only being removed once the trap is in place. Alternatively, the traps may be sold separately, and the compound of the disclosure provided in dispensable format so that an amount may be applied to trap, once the trap is in place. Thus, the present disclosure may provide the compound in a sachet or other dispenser.

### Dispenser

Pheromone compositions can be used in conjunction with a dispenser for release of the composition in a particular environment. Any suitable dispenser known in the art can be used. Examples of such dispensers include but are not limited to, aerosol emitters, hand-applied dispensers, bubble caps comprising a reservoir with a permeable barrier through which pheromones are slowly released, pads, beads, tubes rods, spirals or balls composed of rubber, plastic, leather, cotton, cotton wool, wood or wood products that are impregnated with the pheromone composition. For example, polyvinyl chloride laminates, pellets, granules, ropes or spirals from which the pheromone composition evaporates, or rubber septa. One of skill in the art will be able to select suitable carriers and/or dispensers for the desired mode of application, storage, transport or handling.

In another embodiment, a device may be used that contaminates the male insects with a powder containing the pheromone substance itself. The contaminated males then fly off and provide a source of mating disruption by permeating the atmosphere with the pheromone substance, or by attracting other males to the contaminated males, rather than to real females.

In another embodiment, a device may be used that contaminates the male insects with a powder containing the pheromone substance itself. The contaminated males then fly off and provide a source of mating disruption by permeating the atmosphere with the pheromone substance, or by attracting other males to the contaminated males, rather than to real females.

Retrievable polymeric dispensers are defined as a “solid matrix dispenser” delivering pheromones “at rates less than or equal to 150 grams active ingredient (AI)/acre/year” that is “placed by hand in the field and is of such size and construction that it is readily recognized and retrievable” (40 CFR 180). These dispensers are not in direct contact with crops (chemicals serve as mating attractants).

In another embodiment, hollow fibers may be used which consist of an impermeable, short tube that is sealed at one end and then filled with pheromones. After a short initial burst of pheromones, the emission rate remains fairly constant. Application may require specialized aerial or ground equipment.

In another embodiment, high-emission dispensers may be used which deliver large quantities of pheromones while using fewer dispensers, thus reducing labor costs. Mechanical puffers may be used for mating disruption and confusion. A battery-powered, automatic metered dispenser releases a high emission aerosol or ‘puff’ of pheromone at fixed time intervals (generally every 15 minutes) for a 12-hour period during normal mating time (at night). The labeled use of this product indicates that only two puffers should be placed on every one acre of land; however the number of units required per acre varies depending on land/orchard size and patterns of distribution. The use of puffer systems can produce significant cost savings because less labor is required in comparison to hand application, but, depending on pest pressure and surrounding landscape, applications of additional pheromones along field borders using hand dispensers may be needed.

In some embodiments, alternative pheromone dispensing methods include the aerial or ground application of pheromone-impregnated flakes, and the use of polymer bags filled with large doses of pheromone. Specialized Pheromone and Lure Application Technology (SPLAT™) is a proprietary base matrix formulation of biologically inert materials used to control the release of semiochemicals with or without pesticides. SPLAT™ products include pheromones that prevent the mating and reproduction of lepidopterous insects and can be applied as a spray using hand, aerial, or group equipment. SPLAT™ products for the control of oriental fruit moth, pink bollworm, codling moth, gypsy moth, light brown apple moth, carob moth, and citrus leafminer are commercially available (ISCA Technologies, 2010).

### Behavior Modification

Pheromone compositions prepared according to the methods disclosed herein can be used to control or modulate the behavior of insects. In some embodiments, the behavior of the target insect can be modulated in a tunable manner inter alia by varying the ratio of the pheromone to the positional isomer in the composition such that the insect is attracted to a particular locus but does not contact said locus or such the insect in fact contacts said locus. Thus, in some embodiments, the pheromones can be used to attract insects away from vulnerable crop areas. Accordingly, the disclosure also provides a method for attracting insects to a locus. The method includes administering to a locus an effective amount of the pheromone composition. The method of mating disruption may include periodically monitoring the total number or quantity of the trapped insects. The monitoring may be performed by counting the number of insects trapped for a predetermined period of time such as, for example, daily, Weekly, bi-Weekly, monthly, once-in-three months, or any other time periods selected by the monitor. Such monitoring of the trapped insects may help estimate the population of insects for that particular period, and thereby help determine a particular type and/or dosage of pest control in an integrated pest management system. For example, a discovery of a high insect population can necessitate the use of methods for removal of the insect. Early warning of an infestation in a new habitat can allow action to be taken before the population becomes unmanageable. Conversely, a discovery of a low insect population can lead to a decision that it is sufficient to continue monitoring the population. Insect populations can be monitored regularly so that the insects are only controlled when they reach a certain threshold. This provides cost-effective control of the insects and reduces the environmental impact of the use of insecticides.

### Mating Disruption

Pheromones prepared according to the methods of the disclosure can also be used to disrupt mating. Mating disruption is a pest management technique designed to control insect pests by introducing artificial stimuli (e.g., a pheromone composition as disclosed herein) that confuses the insects and disrupts mating localization and/or courtship, thereby preventing mating and blocking the reproductive cycle.

In many insect species of interest to agriculture, such as those in the order Lepidoptera, females emit an airborne trail of a specific chemical blend constituting that species' sex pheromone. This aerial trail is referred to as a pheromone plume. Males of that species use the information contained in the pheromone plume to locate the emitting female (known as a “calling” female). Mating disruption exploits the male insects' natural response to follow the plume by introducing a synthetic pheromone into the insects' habitat, which is designed to mimic the sex pheromone produced by the female insect. Thus, in some embodiments, the synthetic pheromone utilized in mating disruption is a synthetically derived pheromone composition comprising a pheromone having a chemical structure of a sex pheromone and a positional isomer thereof which is not produced by the target insect.

The general effect of mating disruption is to confuse the male insects by masking the natural pheromone plumes, causing the males to follow “false pheromone trails” at the expense of finding mates, and affecting the males' ability to respond to “calling” females. Consequently, the male population experiences a reduced probability of successfully locating and mating with females, which leads to the eventual cessation of breeding and collapse of the insect infestation. Strategies of mating disruption include confusion, trail-masking and false-trail following. Constant exposure of insects to a high concentration of a pheromone can prevent male insects from responding to normal levels of the pheromone released by female insects. Trail-masking uses a pheromone to destroy the trail of pheromones released by females. False-trail following is carried out by laying numerous spots of a pheromone in high concentration to present the male with many false trails to follow. When released in sufficiently high quantities, the male insects are unable to find the natural source of the sex pheromones (the female insects) so that mating cannot occur.

In some embodiments, a wick or trap may be adapted to emit a pheromone for a period at least equivalent to the breeding season(s) of the midge, thus causing mating disruption. If the midge has an extended breeding season, or repeated breeding season, the present disclosure provides a wick or trap capable of emitting pheromone for a period of time, especially about two weeks, and generally between about 1 and 4 weeks and up to 6 weeks, which may be rotated or replaced by subsequent similar traps. A plurality of traps containing the pheromone composition may be placed in a locus, e.g., adjacent to a crop field. The locations of the traps, and the height of the traps from ground may be selected in accordance with methods known to one skilled in the art. Alternatively, the pheromone composition may be dispensed from formulations such as microcapsules or twist-ties, such as are commonly used for disruption of the mating of insect pests.

### Attract and Kill

In some embodiments, a wick or trap may be adapted to emit a pheromone for a period at least equivalent to the breeding season(s) of the midge, thus causing mating disruption. If the midge has an extended breeding season, or repeated breeding season, the present disclosure provides a wick or trap capable of emitting pheromone for a period of time, especially about two weeks, and generally between about 1 and 4 weeks and up to 6 weeks, which may be rotated or replaced by subsequent similar traps. A plurality of traps containing the pheromone composition may be placed in a locus, e.g., adjacent to a crop field. The locations of the traps, and the height of the traps from ground may be selected in accordance with methods known to one skilled in the art.

The attract and kill method utilizes an attractant, such as a sex pheromone, to lure insects of the target species to an insecticidal chemical, surface, device, etc., for mass-killing and ultimate population suppression, and can have the same effect as mass-trapping. For instance, when a synthetic female sex pheromone is used to lure male pests, e.g., moths, in an attract-and-kill strategy, a large number of male moths must be killed over extended periods of time to reduce matings and reproduction, and ultimately suppress the pest population. The attract-and-kill approach may be a favorable alternative to mass-trapping because no trap-servicing or other frequent maintenance is required. In various embodiments described herein, a recombinant microorganism can co-express (i) a pathway for production of an insect pheromone and (ii) a protein, peptide, oligonucleotide, or small molecule which is toxic to the insect. In this way, the recombinant microorganism can co-produce substances suitable for use in an attract-and-kill approach.

As will be apparent to one of skill in the art, the amount of a pheromone or pheromone composition used for a particular application can vary depending on several factors such as the type and level of infestation; the type of composition used; the concentration of the active components; how the composition is provided, for example, the type of dispenser used; the type of location to be treated; the length of time the method is to be used for; and environmental factors such as temperature, wind speed and direction, rainfall and humidity. Those of skill in the art will be able to determine an effective amount of a pheromone or pheromone composition for use in a given application.

As used herein, an “effective amount” means that amount of the disclosed pheromone composition that is sufficient to affect desired results. An effective amount can be administered in one or more administrations. For example, an effective amount of the composition may refer to an amount of the pheromone composition that is sufficient to attract a given insect to a given locus. Further, an effective amount of the composition may refer to an amount of the pheromone composition that is sufficient to disrupt mating of a particular insect population of interest in a given locality.

### RNAi Delivery and Formulations

In one embodiment, disrupting expression of one or more target genes by RNAi comprises feeding RNAi molecules to one or more pests. Oral delivery of RNAi molecules aims to silence the selected gene after gut-mediated uptake and transport to the insect cells. If oral delivery is efficient, then much higher possibilities exist to formulate an RNAi-based insecticide. For orally delivering RNAi molecules, RNAi molecules should be in vitro synthesized. Then the RNAi molecules are incorporated to the artificial diets of the insects or sprayed on plants which the insects feed on. Delivering RNAi molecules via feeding has several advantages. First, feeding causes little mechanical damage to insects. Further, feeding is convenient for the RNAi manipulation of a large number of individuals. This approach has been successful in at least 15 insect species belonging to seven different orders (Huvenne, H. and Smagghe, G. (2010) Mechanisms of dsRNA uptake in insects and potential of RNAi for pest control: a review. J. Insect Physiol. 56: 227-235).

In another embodiment, RNAi molecules may also be transcribed in bacteria rather than in vitro. Bacterial dsRNA administration is based on the observations of Timmons and Fire (Timmons, L., Fire, A., 1998. Specific interference by ingested dsRNA. Nature 395, 854) which showed that ingestion of bacterially expressed dsRNAs could produce specific and potent genetic interference in C. elegans. This approach uses an RNase III-deficient Escherichia coli strain known as HT115 (DE3) [F-, mcrA, mcrB, IN(rrnD-rrnE)1, rnc14::Tn10 (DE3 lysogen: lavUV5 promoter-T7 polymerase]. In this methodology, the gene of interest is cloned between two T7 promoters on a special RNAi plasmid known as L4440 (T7p, T7p, lacZN, OriF1). The plasmid is transformed in HT115 cells and dsRNA production is achieved after induction with IPTG. The induced cells are then introduced in the worm's growth media and RNAi is achieved after a short period of incubation. Similarly, in insects the IPTG-induced bacteria are incorporated in the insects' artificial diets or they are sprayed in plant organs that insects are feeding on and RNAi is induced after a period of continuous feeding. One potential advantage of this approach is that dsRNA continuously produced in organisms is more stable than dsRNA transcribed in vitro when placed in food. This bacteria-mediated RNAi approach has been successfully applied to other organisms, including a planarian Schmidtea mediterranea (Newmark, P. A., Reddien, P. W., Cebria, F. and Sanchez Alvarado, A. (2003) Ingestion of bacterially expressed double-stranded RNA inhibits gene expression in planarians. Proc. Natl. Acad. Sci. USA 100 (Suppl. 1): 11861-11865) and the lepidopteran insect Spodoptera exigua (Tian, H., Peng, H., Yao, Q., Chen, H., Xie, Q., Tang, B. and Zhang, W. (2009) Developmental control of a lepidopteran pest Spodoptera exigua by ingestion of bacteria expressing dsRNA of a non-midgut gene. PLoS One 4: e6225).

In one embodiment, feeding protocols are modified to use lipid-encapsulated RNAi molecules rather than naked RNAi molecules. Liposomes have been used as nucleic acid transfection media for over 20 years; this approach originated from studies examining the ability of cationic lipids to deliver both DNA and RNA molecules into mouse and human cell lines. Conjugation to lipophilic molecules (cholesterol, bile acids, and long-chain fatty acids) has been shown to increase siRNA uptake into cells and enhance gene silencing in mice. Efficient and selective uptake of these lipid-associated siRNAs depends on interactions with lipoprotein particles, lipoprotein receptors and transmembrane proteins. The efficacy of four commercially available transfection reagents inducing RNAi was evaluated in D. melanogaster. Larvae ingested dsRNA directed against the gus reporter gene encapsulated in different kinds of liposomes for 2 h. Compared to a small (5-8%) reduction by naked dsRNAs, all liposomes facilitated some degree of RNAi effect in the isolated gut tissues (Whyard, S., Singh, A. D. and Wong, S. (2009) Ingested double-stranded RNAs can act as species-specific insecticides. Insect Biochem. Mol. Biol. 39: 824-832).

In one embodiment, the feeding protocol comprises delivering RNAi molecules by a vegetable delivery method. For example, Ghosh et al. (2017) successfully demonstrated the use of a vegetable, green bean, to deliver dsRNA designed to specifically impact and reduce brown marmorated stink bug (BMSB), an insect pest of global importance (Ghosh S K B, Hunter W B, Park A L, Gundersen-Rindal D E (2017) Double strand RNA delivery system for plant-sap-feeding insects. PLoS ONE 12(2): e0171861.). The selection of green beans as the vegetable for delivery relied on the ease of availability, cost, and natural attractiveness to the insect. BMSB is a phloem-feeder causing damage by piercing and sucking from the vascular tissues of fruits and vegetables. The plant vascular system was suitable for uptake of in vitro synthesized dsRNA, providing efficient delivery to the animal as demonstrated by reducing BMSB-specific JHAMT and Vg (vitellogenin) gene expression in BMSB tissues.

In one embodiment, disrupting expression of one or more target genes by RNAi comprises growing transgenic plants expressing RNAi molecules in the field plot as a source of food for the one or more pests. To exploit the feasible and practical benefit of RNAi in field control of pests, plants are a good choice for RNAi molecule, such as dsRNA, production. First, plants are the host and food source of herbivorous insects. Second, plants have tons of biomass and could accumulate a large amount of RNAi molecules, such as dsRNAs, to provoke the RNAi response. And third, RNAi molecules can be continuously produced under varying environmental conditions. The RNAi molecules could be produced in plants under universal or tissue-specific promoters, as well as under constitutive or inducible promoters. The observation that genetically modified plants expressing dsRNAs targeting specific insect genes could induce RNAi in the insect pests was first reported in independent publications of Baum et al. (Baum et al. 2007. Control of coleopteran insect pests through RNA interference. Nature biotechnology 25: 1322-1326) and Mao et al. (Mao, Y. B. et al. (2007) Silencing a cotton bollworm P450 monooxygenase gene by plant-mediated RNAi impairs larval tolerance of gossypol. Nature Biotechnology 25: 1307-1313). Baum et al. showed that corn plants expressing hairpin dsRNAs that target the A subunit of ATPase gene in the western corn rootworm were significantly protected by the damage caused by this pest (Baum et al. 2007). Furthermore, Arabidopsis plants expressing dsRNA hairpins targeting the cytochrome P450 monooxygenase gene in the corn pest H. armigera led to decreased resistance to the sesquiterpene gossypol to the feeding insects (Mao et al. 2007).

In some embodiments, the RNAi molecules can be produced in chloroplasts of plants. Since small RNAs can be produced in bacteria and the plastid genome is of bacterial origin, it is possible to engineer chloroplasts to produce RNAi molecules. Expressing foreign genes in chloroplast offers several advantages over nuclear expression. First, chloroplast transformation may result in high expression levels due to numerous copies of chloroplasts in a cell. Secondly, traits encoded by chloroplast are predominantly maternally inherited in most plants, so that the transgene is less likely to be transmitted to non-transgenic plants.

In one embodiment, disrupting expression of one or more target genes by RNAi comprises infecting the one or more pests with one or more viruses expressing RNAi molecules. The use of viruses is a less common methodology to transfer dsRNAs into the insect tissues. Virus-mediated-RNAi involves the expression of an RNAi transgene into a virus which is then used to infect the insect cell or a tissue in order to express RNAi molecules intracellularly. This methodology has not been used extensively because of the general viral interference with normal cell physiology; for instance, baculoviruses cause high lethality and potential phenotypes could not be distinguished between dsRNA-producing and control viruses. In addition, viruses can produce inhibitors of RNAi, thereby lowering silencing efficiency. In order to successfully distinguish effects of virus-mediated RNAi, wild-type viruses should be somehow inactivated or at least should not cause highly toxic effects in the insect host. The first report of successful viral dsRNA delivery was made by Haj os et al. (Haj os JP, Vermunt A M, Zuidema D, Kulcsar P, Varj as L, de Kort C A, Zavodszky P, Vlak J M. Dissecting insect development: baculovirus-mediated gene silencing in insects. Insect Mol Biol. 1999; 8(4):539-544). In this paper, researchers used a recombinant baculovirus, Autographa californica multicapsid nucleopolyhedrovirus (AcMNPV), to express Heliothis virescens juvenile hormone esterase (JHE) gene in antisense orientation, driven by the viral p10 promoter. Infection with this recombinant virus greatly reduced the hemolymph JHE levels and resulted in aberrant morphogenesis of final-instar H. virescens larvae. This was the first time that baculovirus-mediated gene silencing could be accomplished and utilized to dissect insect development and to design a new class of baculovirus-based insecticides.

In one embodiment, disrupting expression of one or more target genes by RNAi comprises spraying RNAi molecules in the field plot containing one or more pests. In some embodiments, the RNAi molecules may be directly sprayed onto the one or more pests or sprayed on the field plot. In some embodiments, the RNAi molecules are sprayed on plants or plant parts in the field plot, which are a source of food for the one or more pests. dsRNA soaking was first introduced in nematodes, and then it was used in insect studies. To test the role of AmSid-I in the systemic effect of RNAi, the honey bee Toll-related receptor 18W gene was silenced by the dsRNA feeding and/or soaking delivery method. The expression levels of AmSid-I and Am-18w were measured using real-time polymerase chain reaction (PCR). A 3.4-fold increase in expression of AmSid-I was observed at 26 h. In contrast, Am-18w gene expression decreased approximately 60-fold at 30 h. High mortality and morphological abnormalities were also seen due to gene silencing (Aronstein, K., Pankiw, T. and Saldivar, E. (2006) SID-I is implicated in systemic gene silencing in the honey bee. Journal of Apicultural Research, 45, 20-24). The soaking strategy was successfully practiced in protecting plants against viral diseases by spraying bacteria expressing dsRNA. Two fragments of the Sugarcane Mosaic Virus (SCMV) CP (coat protein) gene dsRNA were expressed by Escherichia coli HT115. The crude extracts containing large amounts of dsRNA were sprayed on to the plants and result confirmed preventative efficacy. The results provided a valuable tool for plant viral control using dsRNA spraying (Gan, D., Zhang, J., Jiang, H., Jiang, T., Zhu, S. and Cheng, B. (2010) Bacterially expressed dsRNA protects maize against SCMV infection. Plant Cell Reports, 29, 1261-1268).

The Aronstein and Gan reports provided new revelations on whether dsRNA can pass through the body wall, stoma and intersegmental membrane to exercise the role of RNAi. If dsRNA can permeate the insect body wall and enter into the body cavity, RNAi can be applied in the field similar to traditional insecticides by spraying dsRNA on to the body of insects. Spraying experiments have been designed that deal with the Asian corn borer, Ostrinia furnalalis. Results confirmed that the spray method can lead to gene-specific RNAi, and lead to larval lethality or developmental disorders. The results also demonstrated that spraying can achieve a continuous supply of dsRNA and greatly improve target pest mortality (Wang, Y. B., Zhang, H., Li, H. C. and Miao, X. X. (2011) Second generation sequencing supply an effective way to screen RNAi targets in large scale for potential application in pest insect control. PLoS ONE, 6(4), e18644). Spraying can be a viable approach if dsRNA can be cheaply mass produced, especially when dsRNA can reduce the pest population faster than conventional pesticides. In fact, delivering dsRNA by spraying on crop plants fits with the traditional habits of insecticide delivery methods.

In one embodiment, disrupting expression of one or more target genes by RNAi comprises providing nanoparticles comprised of RNAi molecules to one or more pests. In case of genetic material, delivery systems face challenges such as limited host range, transportation across cell membrane and trafficking to the nucleus. Nanomaterials hold great promise regarding their application in plant protection and nutrition due to their size-dependent qualities, high surface-to-volume ratio and unique optical properties. Nanoparticles (NPs) are particles having one or more dimensions on the order of 100 nm or less. NPs are also referred to as colloidal particulate systems with size ranging between 10 and 1000 nm. A wide variety of materials are used to make NPs, such as metal oxides, ceramics, silicates, magnetic materials, semiconductor quantum dots (QDs), lipids, polymers, dendrimers and emulsions. Polymers display controlled release of ingredients, a character useful for developing polymeric NPs as agrochemical carriers. Metal nanoparticles display size dependent properties such as magnetism (magnetic NPs), fluorescence (QDs) or photocatalytic degradation (metal oxide NPs) that have biotechnological applications in sensor development, agrochemical degradation and soil remediation.

In some embodiments, disrupting expression of one or more target genes by RNAi comprises providing sheet-like clay nanoparticles comprised of RNAi molecules to one or more pests. Mitter et al. (2017) demonstrated that dsRNA can be loaded on designer, non-toxic, degradable, layered double hydroxide (LDH) clay nanosheets (Mitter, N et al. (2017) Clay nanosheets for topical delivery of RNAi for sustained protection against plant viruses. Nature plants 3: 16207). LDH materials occur naturally as a result of precipitation in saline water bodies or through the weathering of basalts. Once loaded on LDH, the dsRNA does not wash off, shows sustained release and can be detected on sprayed leaves even 30 days after application. Mitter et al. show the degradation of LDH, dsRNA uptake in plant cells and silencing of homologous RNA on topical application. Significantly, a single spray of dsRNA loaded on LDH (BioClay) afforded virus protection for at least 20 days when challenged on sprayed and newly emerged unsprayed leaves. The clay nanosheets offer an environmentally sustainable and easy to adopt topical spray for delivery of RNAi.

In some embodiments, the RNAi molecules are formulated to be used as seed treatments.

In one embodiment, disrupting expression of one or more target genes by RNAi comprises providing chitosan nanoparticles comprised of RNAi molecules to one or more pests. Chitosan has emerged as one of the most promising polymers for the efficient delivery of agrochemicals and micronutrients in nanoparticles. The enhanced efficiency and efficacy of nanoformulations are due to higher surface area, induction of systemic activity due to smaller particle size and higher mobility, and lower toxicity due to elimination of organic solvents in comparison to conventionally used pesticides and their formulations. Chitosan nanoparticles have been investigated as a carrier for active ingredient delivery for various applications owing to their biocompatibility, biodegradability, high permeability, cost-effectiveness, non-toxicity and excellent film forming ability (S. K. Shukla, A. K. Mishra, O. A. Arotiba, B. B. Mamba (2013) Int. J. Biol. Macromol. 59: 46-58). Over the past three decades, various procedures like cross-linking, emulsion formation, coacervation, precipitation and self-assembly, etc. have been employed to synthesize chitosan nanoparticles. Chitosan has also known for its broad spectrum antimicrobial and insecticidal activities. Further, it is biodegradable giving non-toxic residues with its rate of degradation corresponding to molecular mass and degree of deacetylation. Because of its cationic nature, chitosan can make complex with siRNA easily and forms nanoparticles. Several reports indicate the application of chitosan nanoparticle-entrapped siRNA as a carrier for siRNA delivery (H. Katas, H. O. Alpar (2006) J. Control. Release 115: 216-225; J. Malmo, H. Sørgård, K. M. Varum, S. P. Strand (2012) J. Control. Release 158: 261-268; H. Ragelle, G. Vandermeulen, V. Préa (2013) J. Control. Release 172: 207-218). Chitosan nanoparticles have successfully delivered dsRNA (against chitin synthase genes) in stabilized form, to mosquito larvae via feeding (X. Zhang, J. Zhang, K. Y. Zhu (2010) Insect Mol. Biol. 19: 683-693). Chitosan nanoparticles may be efficient in dsRNA delivery due to their efficient binding with RNA, protection and the ability to penetrate through the cell membrane.

### Helicoverpa

Helicoverpa is a genus of moth in the Noctuidae family. Species in the Helicoverpa genus include H. armigera, H. assulta, H. atacamae, H. fletcheri, H. gelotopoeon, H. hardwicki, H. hawaiiensis, H. helenae, H. pallida, H. prepodes, H. punctigera, H. titicacae, H. toddi and H. zea. H. confusa and H. minuta are two Helicoverpa species that are extinct.

Helicoverpa armigera

H. armigera is commonly known as the cotton bollworm when found outside the United States, or alternatively the “Old World (African) bollworm”. The larvae of this moth feed on a wide range of plants, including economically important cultivated crops. This species is widespread in central and southern Europe, temperate Asia, Africa, Australia and Oceania, and has also recently been confirmed to have successfully invaded Brazil and the US. It is a migrant species, able to reach Scandinavia and other northern territories. The female cotton bollworm can lay several hundred eggs, distributed on various parts of the plant. Under favorable conditions, the eggs can hatch into larvae within three days and the whole life cycle can be completed in just over a month.

The cotton bollworm is a highly polyphagous species, being able to feed on many crops. It is a major pest in cotton. The most important crop hosts are tomato, cotton, pigeon pea, chickpea, sorghum and cowpea. Other hosts include groundnut, okra, peas, field beans, soybeans, lucerne, Phaseolus spp., other Leguminosae, tobacco, potatoes, maize, flax, Dianthus, Rosa, Pelargonium, Chrysanthemum, Lavandula angustifolia, a number of fruit trees, forest trees and a range of vegetable crops. In Russia and adjacent countries, the larvae populate more than 120 plant species, favoring Solanum, Datura, Hyoscyamus, Atriplex and Amaranthus genera.

The greatest damage is caused to cotton, tomatoes, maize, chick peas, alfalfa and tobacco. In cotton crops, blooms that have been attacked may open prematurely and stay fruitless. When the bolls are damaged, some will fall off and others will fail to produce lint or produce lint of an inferior quality. Secondary infections by fungi and bacteria are common and may lead to rotting of fruits. Injury to the growing tips of plants may disturb their development, delay maturity and cause fruits to drop.

Helicoverpa zea (Formerly Heliothis zea)

Helicoverpa zea (or Heliothis zea) is also commonly known as the corn earworm and the cotton bollworm in the United States. Thus, the species should not be confused with the aforementioned H. armigera, which is given the common name “cotton bollworm” outside of the United States and “old world bollworm” within the United States. Corn earworm is found throughout North America except for northern Canada and Alaska. In the eastern United States, corn earworm does not normally overwinter successfully in the northern states. It is known to survive as far north as about 40 degrees north latitude, or about Kansas, Ohio, Virginia, and southern New Jersey, depending on the severity of winter weather. However, it is highly dispersive, and routinely spreads from southern states into northern states and Canada. Thus, areas have overwintering, both overwintering and immigrant, or immigrant populations, depending on location and weather. In the relatively mild Pacific Northwest, corn earworm can overwinter at least as far north as southern Washington.

Helicoverpa zea is active throughout the year in tropical and subtropical climates, but becomes progressively more restricted to the summer months with increasing latitude. In northeastern states dispersing adults may arrive as early as May or as late as August due to the vagaries associated with weather; thus, their population biology is variable. The number of generations is usually reported to be one in northern areas such as most of Canada, Minnesota, and western New York; two in northeastern states; two to three in Maryland; three in the central Great Plains; and northern California; four to five in Louisiana and southern California; and perhaps seven in southern Florida and southern Texas. The life cycle can be completed in about 30 days.

Egg:

Eggs are deposited singly, usually on leaf hairs and corn silk. The egg is pale green when first deposited, becoming yellowish and then gray with time. The shape varies from slightly dome-shaped to a flattened sphere, and measures about 0.5 to 0.6 mm in diameter and 0.5 mm in height. Fecundity ranges from 500 to 3000 eggs per female. The eggs hatch in about three to four days.

Larva:

Upon hatching, larvae wander about the plant until they encounter a suitable feeding site, normally the reproductive structure of the plant. Young larvae are not cannibalistic, so several larvae may feed together initially. However, as larvae mature they become very aggressive, killing and cannibalizing other larvae. Consequently, only a small number of larvae are found in each ear of corn. Normally, corn earworm displays six instars, but five is not uncommon and seven to eight have been reported. Mean head capsule widths are 0.29, 0.47, 0.77, 1.30, 2.12, and 3.10 mm, respectively, for instars 1 to 6. Larval lengths are estimated at 1.5, 3.4, 7.0, 11.4, 17.9, and 24.8 mm, respectively. Development time averaged 3.7, 2.8, 2.2, 2.2, 2.4, and 2.9 days, respectively, for instars 1 to 6 when reared at 25° C. Butler (Butler Jr. G. D. (1976) Bollworm: development in relation to temperature and larval food. Environmental Entomology 5: 520-522) cultured earworm on corn at several temperatures, reporting total larval development times of 31.8, 28.9, 22.4, 15.3, 13.6, and 12.6 days at 20.0, 22.5, 25.0, 30.0, 32.0, and 34.0° C., respectively.

The larva is variable in color. Overall, the head tends to be orange or light brown with a white net-like pattern, the thoracic plates black, and the body brown, green, pink, or sometimes yellow or mostly black. The larva usually bears a broad dark band laterally above the spiracles, and a light yellow to white band below the spiracles. A pair of narrow dark stripes often occurs along the center of the back. Close examination reveals that the body bears numerous black thorn-like microspines. These spines give the body a rough feel when touched. The presence of spines and the light-colored head serve to distinguish corn earworm from fall armyworm, Spodoptera frugiperda (J. E. Smith), and European corn borer, Ostrinia nubilalis (Hubner). These other common corn-infesting species lack the spines and have dark heads. Tobacco budworm, Heliothis virescens (Fabricius), is a closely related species in which the late instar larvae also bear microspines. Although it is easily confused with corn earworm, it rarely is a vegetable pest and never feeds on corn. Close examination reveals that in tobacco budworm larvae the spines on the tubercles of the first, second, and eighth abdominal segments are about half the height of the tubercles, but in corn earworm the spines are absent or up to one-fourth the height of the tubercle. Younger larvae of these two species are difficult to distinguish, but Neunzig (1964) give a key to aid in separation (Neunzig H. H. (1964) The eggs and early-instar larvae of Heliothis zea and Heliothis virescens (Lepidoptera: Noctuidae). Annals of the Entomological Society of America 57: 98-102).

Pupa:

Mature larvae leave the feeding site and drop to the ground, where they burrow into the soil and pupate. The larva prepares a pupal chamber 5 to 10 cm below the surface of the soil. The pupa is mahogany-brown in color, and measures 17 to 22 mm in length and 5.5 mm in width. Duration of the pupal stage is about 13 days (range 10 to 25) during the summer.

Adult:

As with the larval stage, adults are quite variable in color. The forewings of the moths usually are yellowish brown in color, and often bear a small dark spot centrally. The small dark spot is especially distinct when viewed from below. The forewing also may bear a broad dark transverse band distally, but the margin of the wing is not darkened. The hind wings are creamy white basally and blackish distally, and usually bear a small dark spot centrally. The moth measures 32 to 45 mm in wingspan. Adults are reported to live for five to 15 days, but may survive for over 30 days under optimal conditions. The moths are principally nocturnal, and remain active throughout the dark period. During the daylight hours they usually hide in vegetation, but sometimes can be seen feeding on nectar. Oviposition commences about three days after emergence, continuing until death. Fresh-silking corn is highly attractive for oviposition but even ears with dry silk will receive eggs. Fecundity varies from about 500 to 3000 eggs, although feeding is a prerequisite for high levels of egg production. Females may deposit up to 35 eggs per day.

Corn earworm has a wide host range; hence, it is also known as “tomato fruitworm,” “sorghum headworm,” “vetchworm,” and “cotton bollworm.” In addition to corn and tomato, perhaps its most favored vegetable hosts, corn earworm also attacks artichoke, asparagus, cabbage, cantaloupe, collard, cowpea, cucumber, eggplant, lettuce, lima bean, melon, okra, pea, pepper, potato, pumpkin, snap bean, spinach, squash, sweet potato, and watermelon. Not all are good hosts, however. Harding, for example, studied relative suitability of crops and weeds in Texas, and reported that although corn and lettuce were excellent larval hosts, tomato was merely a good host, and broccoli and cantaloupe were poor (Harding J. A. (1976) Heliothis spp.: seasonal occurrence, hosts and host importance in the lower Rio Grande Valley. Environmental Entomology 5: 666-668). Other crops injured by corn earworm include alfalfa, clover, cotton, flax, oat, millet, rice, sorghum, soybean, sugarcane, sunflower, tobacco, vetch, and wheat. Among field crops, sorghum is particularly favored. Cotton is frequently reported to be injured, but this generally occurs only after more preferred crops have matured. Fruit and ornamental plants may be attacked, including ripening avocado, grape, peaches, pear, plum, raspberry, strawberry, carnation, geranium, gladiolus, nasturtium, rose, snapdragon, and zinnia. In studies conducted in Florida, Martin et al. found corn earworm larvae on all 17 vegetable and field crops studied, but corn and sorghum were most favoured (Martin P. B. et al. (1976) Relative abundance and host preferences of cabbage looper, soybean looper, tobacco budworm, and corn earworm on crops grown in northern Florida. Environmental Entomology 5: 878-882). In cage tests earworm moths preferred to oviposit on tomato over a selection of several other vegetables that did not include corn.

Such weeds as common mallow, crown vetch, fall panicum, hemp, horsenettle, lambsquarters, lupine, morningglory, pigweed, prickly sida, purslane, ragweed, Spanish needles, sunflower, toadflax, and velvetleaf, have been reported to serve as larval. However, Harding (1976) rated only sunflower as a good weed host relative to 10 other species in a study conducted in Texas. Stadelbacher indicated that crimson clover and winter vetch, which may be both crops and weeds, were important early season hosts in Mississippi (Stadelbacher E. A. (1981) Role of early-season wild and naturalized host plants in the buildup of the F1 generation of Heliothis zea and H. virescens in the Delta of Mississippi. Environmental Entomology 10: 766-770). He also indicated that cranesbill species were particularly important weed hosts in this area. In North Carolina, especially important wild hosts were toadflax and deergrass (Neunzig H. H. (1963) Wild host plants of the corn earworm and the tobacco budworm in eastern North Carolina. Journal of Economic Entomology 56: 135-139).

Adults collect nectar or other plant exudates from a large number of plants. Trees and shrub species are especially frequented. Among the hosts are Citrus, Salix, Pithecellobium, Quercus, Betula, Prunus, Pyrus and other trees, but also alfalfa; red and white clover; milkweed, and Joe-Pye weed and other flowering plants.

Corn earworm is considered by some to be the most costly crop pest in North America. It is more damaging in areas where it successfully overwinters, however, because in northern areas it may arrive too late to inflict extensive damage. It often attacks valuable crops, and the harvested portion of the crop. Thus, larvae often are found associated with such plant structures as blossoms, buds, and fruits. When feeding on lettuce, larvae may burrow into the head. On corn, its most common host, young larvae tend to feed on silks initially, and interfere with pollination, but eventually they usually gain access to the kernels. They may feed only at the tip, or injury may extend half the length of the ear before larval development is completed. Such feeding also enhances development of plant pathogenic fungi. If the ears have not yet produced silk, larvae may burrow directly into the ear. They usually remain feeding within a single ear of corn, but occasionally abandon the feeding site and search for another. Larvae also can damage whorl-stage corn by feeding on the young, developing leaf tissue. Survival is better on more advanced stages of development, however. On tomato, larvae may feed on foliage and burrow in the stem, but most feeding occurs on the tomato fruit. Larvae commonly begin to burrow into a fruit, feed only for a short time, and then move on to attack another fruit. Tomato is more susceptible to injury when corn is not silking; in the presence of corn, moths will preferentially oviposit on fresh corn silk. Other crops such as bean, cantaloupe, cucumber, squash, and pumpkin may be injured in a manner similar to tomato, and also are less likely to be injured if silking corn is nearby.

Although numerous natural enemies have been identified, they usually are not effective at causing high levels of earworm mortality or preventing crop injury. For example, in a study conducted in Texas, Archer and Bynum (1994) reported less than 1% of the larvae were parasitized or infected with disease (Archer T. L. and Bynum Jr. E. D. (1994) Corn earworm (Lepidoptera: Noctuidae) biology on food corn on the High Plains. Environmental Entomology 23: 343-348). However, eggs may be heavily parasitized. Trichogramma spp. (Hymenoptera: Trichogrammatidae), and to a lesser degree Telenomus spp. (Hymenoptera: Scelionidae), are common egg parasitoids. Common larval parasitoids include Cotesia spp., and Microplitis croceipes (Cresson) (all Hymenoptera: Braconidae); Campoletis spp. (Hymenoptera: Ichneumonidae); Eucelatoria armigera (Coquillett) and Archytas marmoratus (Townsend) (Diptera: Tachinidae).

General predators often feed on eggs and larvae of corn earworm; over 100 insect species have been observed to feed on H. zea. Among the common predators are ladybird beetles such as convergent lady beetle, Hippodamia convergens Guerin-Meneville, and Coleomegilla maculata DeGeer (both Coleoptera: Coccinellidae); softwinged flower beetles, Collops spp. (Coleoptera: Melyridae); green lacewings, Chrysopa and Chrysoperla spp. (Neuroptera: Chrysopidae); minute pirate bug, Orius tristicolor (White) (Hemiptera: Anthocoridae); and big-eyed bugs, Geocoris spp. (Hemiptera: Lygaeidae). Birds can also feed on earworms, but rarely are adequately abundant to be effective.

Within-season mortality during the pupal stage seems to be, and although overwintering mortality is often very high the mortality is due to adverse weather and collapse of emergence tunnels rather than to natural enemies. In Texas, Steinernema riobravis (Nematoda: Steinernematidae) has been found to be an important mortality factor of prepupae and pupae, but this parasitoid is not yet generally distributed. Similarly, Heterorhabditis heliothidis (Nematoda: Heterorhabditidae) has been found parasitizing corn earworm in North Carolina, but it has not been found widely. Both of the latter species are being redistributed, and can be produced commercially, so in the future they may assume greater importance in natural regulation of earworm populations.

Epizootics caused by pathogens may erupt when larval densities are high. The fungal pathogen Nomuraea rileyi and the Helicoverpa zea nuclear polyhedrosis virus are commonly involved in outbreaks of disease, but the protozoan Nosema heliothidis and other fungi and viruses also have been observed.

Sampling:

Eggs and larvae often are not sampled on corn because eggs are very difficult to detect, and larvae burrow down into the silks, out of the reach of insecticides, soon after hatching.

Moths can be monitored with blacklight and pheromone traps. Both sexes are captured in light traps, whereas only males are attracted to the sex pheromone. Both trap types give an estimate of when moths invade or emerge, and relative densities, but pheromone traps are easier to use because they are selective. The pheromone is usually used in conjunction with an inverted cone-type trap. Generally, the presence of five to 10 moths per night is sufficient to stimulate pest control practices.

Insecticides:

Corn fields with more than 5% of the plants bearing new silk are susceptible to injury if moths are active. Insecticides are usually applied to foliage in a liquid formulation, with particular attention to the ear zone, because it is important to apply insecticide to the silk. Insecticide applications are often made at two to six day intervals, sometimes as frequently as daily in Florida. Because it is treated frequently, and over a wide geographic area, corn earworm has become resistant to many insecticides. Susceptibility to Bacillus thuringiensis also varies, but the basis for this variation in susceptibility is uncertain. Mineral oil, applied to the corn silk soon after pollination, has insecticidal effects. Application of about 0.75 to 1.0 ml of oil five to seven days after silking can provide good control in the home garden.

Cultural Practices:

Trap cropping is often suggested for this insect; the high degree of preference by ovipositing moths for corn in the green silk stage can be used to lure moths from less preferred crops. Lima beans also are relatively attractive to moths, at least as compared to tomato. However, it is difficult to maintain attractant crops in an attractive stage for protracted periods. In southern areas where populations develop first on weed hosts and then disperse to crops, treatment of the weeds through mowing, herbicides, or application of insecticides can greatly ameliorate damage on nearby crops. In northern areas, it is sometimes possible to plant or harvest early enough to escape injury. Throughout the range of this insect, population densities are highest, and most damaging, late in the growing season. Tillage, especially in the autumn, can significantly reduce overwintering success of pupae in southern locations.

Biological Control:

The bacterium Bacillus thuringiensis, and steinernematid nematodes provide some suppression. Entomopathogenic nematodes, which are available commercially, provide good suppression of developing larvae if they are applied to corn silk; this has application for home garden production of corn if not commercial production (Purcell M. et al. (1992) Biological control of Helicoverpa zea (Lepidoptera: Noctuidae) with Steinernema carpocapsae (Rhabditida: Steinernematidae) in corn used as a trap crop. Environmental Entomology 21: 1441-1447). Soil surface and subsurface applications of nematodes also can affect earworm populations because larvae drop to the soil to pupate (Cabanillas H. E. and Raulston J. R. (1996) Evaluation of Steinernema riobravis, S. carpocapsae, and irrigation timing for the control of corn earworm, Helicoverpa zea. Journal of Nematology 28: 75-82). This approach may have application for commercial crop protection, but larvae must complete their development before they are killed, so some crop damage ensues.

Trichogramma spp. (Hymenoptera: Trichogrammatidae) egg parasitoids have been reared and released for suppression of H. zea in several crops. Levels of parasitism averaging 40 to 80% have been attained by such releases in California and Florida, resulting in fruit damage levels of about 3% (Oatman E. R. and Platner G. R. (1971) Biological control of the tomato fruitworm, cabbage looper, and hornworms on processing tomatoes in southern California, using mass releases of Trichogramma pretiosum. Journal of Economic Entomology 64: 501-506). The host crop seems to affect parasitism rates, with tomato being an especially suitable crop for parasitoid releases (Martin P. B. et al. (1976) Parasitization of two species of Plusiinae and Heliothis spp. after releases of Trichogramma pretiosum in seven crops. Environmental Entomology 5: 991-995).

Host Plant Resistance:

Numerous varieties of corn have been evaluated for resistance to earworm, and some resistance has been identified in commercially available corn. Resistance is derived from physical characteristics such as husk tightness and ear length, which impede access by larvae to the ear kernels, or chemical factors such as maysin, which inhibit larval growth. Host plant resistance thus far is not completely adequate to protect corn from earworm injury, but it may prove to be a valuable component of multifaceted pest management programs. Varieties of some crops are now available that incorporate Bacillus thuringiensis toxin, which reduces damage by H. zea.

### Spodoptera

Spodoptera is a genus of moths of the family Noctuidae. About 30 species are distributed across six continents. Many are insect pests, and the larvae are sometimes called armyworms.

Spodoptera frugiperda

Spodoptera frugiperda, commonly known as fall armyworm, is native to the tropical regions of the western hemisphere from the United States to Argentina. It normally overwinters successfully in the United States only in southern Florida and southern Texas. The fall armyworm is a strong flier, and disperses long distances annually during the summer months. It is recorded from virtually all states east of the Rocky Mountains. However, as a regular and serious pest, its range tends to be mostly the southeastern states. The life cycle is completed in about 30 days during the summer, but 60 days in the spring and autumn, and 80 to 90 days during the winter. The number of generations occurring in an area varies with the appearance of the dispersing adults. The ability to diapause is not present in this species. In Minnesota and New York, where fall armyworm moths do not appear until August, there may be but a single generation. The number of generations is reported to be one to two in Kansas, three in South Carolina, and four in Louisiana. In coastal areas of north Florida, moths are abundant from April to December, but some are found even during the winter months.

Egg:

The egg is dome shaped; the base is flattened and the egg curves upward to a broadly rounded point at the apex. The egg measures about 0.4 mm in diameter and 0.3 m in height. The number of eggs per mass varies considerably but is often 100 to 200, and total egg production per female averages about 1500 with a maximum of over 2000. The eggs are sometimes deposited in layers, but most eggs are spread over a single layer attached to foliage. The female also deposits a layer of grayish scales between the eggs and over the egg mass, imparting a furry or moldy appearance. Duration of the egg stage is only two to three days during the summer months.

Larvae:

There usually are six instars in fall armyworm. Head capsule widths are about 0.35, 0.45, 0.75, 1.3, 2.0, and 2.6 mm, respectively, for instars 1-6. Larvae attain lengths of about 1.7, 3.5, 6.4, 10.0, 17.2, and 34.2 mm, respectively, during these instars. Young larvae are greenish with a black head, the head turning orangish in the second instar. In the second, but particularly the third instar, the dorsal surface of the body becomes brownish, and lateral white lines begin to form. In the fourth to the sixth instars the head is reddish brown, mottled with white, and the brownish body bears white subdorsal and lateral lines. Elevated spots occur dorsally on the body; they are usually dark in color, and bear spines. The face of the mature larva is also marked with a white inverted “Y” and the epidermis of the larva is rough or granular in texture when examined closely. However, this larva does not feel rough to the touch, as does corn earworm, Helicoverpa zea (Boddie), because it lacks the microspines found in the similar-appearing corn earworm. In addition to the typical brownish form of the fall armyworm larva, the larva may be mostly green dorsally. In the green form, the dorsal elevated spots are pale rather than dark. Larvae tend to conceal themselves during the brightest time of the day. Duration of the larval stage tends to be about 14 days during the summer and 30 days during cool weather. Mean development time was determined to be 3.3, 1.7, 1.5, 1.5, 2.0, and 3.7 days for instars 1 to 6, respectively, when larvae were reared at 25° C. (Pitre H. N. and Hogg D. B. (1983) Development of the fall armyworm on cotton, soybean and corn. Journal of the Georgia Entomological Society 18: 187-194).

Pupa:

Pupation normally takes place in the soil, at a depth 2 to 8 cm. The larva constructs a loose cocoon, oval in shape and 20 to 30 mm in length, by tying together particles of soil with silk. If the soil is too hard, larvae may web together leaf debris and other material to form a cocoon on the soil surface. The pupa is reddish brown in color, and measures 14 to 18 mm in length and about 4.5 mm in width. Duration of the pupal stage is about eight to nine days during the summer, but reaches 20 to 30 days during the winter in Florida. The pupal stage of fall armyworm cannot withstand protracted periods of cold weather. For example, Pitre and Hogg (1983) studied winter survival of the pupal stage in Florida, and found 51 percent survival in southern Florida, but only 27.5 percent survival in central Florida, and 11.6 percent survival in northern Florida.

Adult:

The moths have a wingspan of 32 to 40 mm. In the male moth, the forewing generally is shaded gray and brown, with triangular white spots at the tip and near the center of the wing. The forewings of females are less distinctly marked, ranging from a uniform grayish brown to a fine mottling of gray and brown. The hind wing is iridescent silver-white with a narrow dark border in both sexes. Adults are nocturnal, and are most active during warm, humid evenings. After a preoviposition period of three to four days, the female normally deposits most of her eggs during the first four to five days of life, but some oviposition occurs for up to three weeks. Duration of adult life is estimated to average about 10 days, with a range of about seven to 21 days.

A comprehensive account of the biology of fall armyworm was published by Luginbill (Luginbill P. (1928) The Fall Armyworm. USDA Technical Bulletin 34. 91 pp.), and an informative synopsis by Sparks (Sparks A. N. (1979) A review of the biology of the fall armyworm. Florida Entomologist 62: 82-87). Ashley et al. (1989) presented an annotated bibliography (Ashley T. R. et al. (1989) The fall armyworm: a bibliography. Florida Entomologist 72: 152-202). A sex pheromone has been described (Sekul A. A. and Sparks A. N. (1976) Sex attractant of the fall armyworm moth. USDA Technical Bulletin 1542. 6 PP.).

This species seemingly displays a very wide host range, with over 80 plants recorded, but clearly prefers grasses. The most frequently consumed plants are field corn and sweet corn, sorghum, Bermudagrass, and grass weeds such as crabgrass, Digitaria spp. When the larvae are very numerous they defoliate the preferred plants, acquire an “armyworm” habit and disperse in large numbers, consuming nearly all vegetation in their path. Many host records reflect such periods of abundance, and are not truly indicative of oviposition and feeding behavior under normal conditions. Field crops are frequently injured, including alfalfa, barley, Bermuda grass, buckwheat, cotton, clover, corn, oat, millet, peanut, rice, ryegrass, sorghum, sugarbeet, sudangrass, soybean, sugarcane, timothy, tobacco, and wheat. Among vegetable crops, only sweet corn is regularly damaged, but others are attacked occasionally. Other crops sometimes injured are apple, grape, orange, papaya, peach, strawberry and a number of flowers. Weeds known to serve as hosts include bentgrass, Agrostis sp.; crabgrass, Digitaria spp.; Johnson grass, Sorghum halepense; morning glory, Ipomoea spp.; nutsedge, Cyperus spp.; pigweed, Amaranthus spp.; and sandspur, Cenchrus tribuloides.

There is some evidence that fall armyworm strains exist, based primarily on their host plant preference. One strain feeds principally on corn, but also on sorghum, cotton and a few other hosts if they are found growing near the primary hosts. The other strain feeds principally on rice, Bermudagrass, and Johnson grass. Larvae cause damage by consuming foliage. Young larvae initially consume leaf tissue from one side, leaving the opposite epidermal layer intact. By the second or third instar, larvae begin to make holes in leaves, and eat from the edge of the leaves inward. Feeding in the whorl of corn often produces a characteristic row of perforations in the leaves. Larval densities are usually reduced to one to two per plant when larvae feed in close proximity to one another, due to cannibalistic behavior. Older larvae cause extensive defoliation, often leaving only the ribs and stalks of corn plants, or a ragged, torn appearance. Marenco et al. (1992) studied the effects of fall armyworm injury to early vegetative growth of sweet corn in Florida (Marenco R. J. et al. (1992) Sweet corn response to fall armyworm (Lepidoptera: Noctuidae) damage during vegetative growth. Journal of Economic Entomology 85: 1285-1292). They reported that the early whorl stage was least sensitive to injury, the midwhorl stage intermediate, and the late whorl stage was most sensitive to injury. Further, they noted that mean densities of 0.2 to 0.8 larvae per plant during the late whorl stage could reduce yield by 5 to 20 percent. Larvae also will burrow into the growing point (bud, whorl, etc.), destroying the growth potential of plants, or clipping the leaves. In corn, they sometimes burrow into the ear, feeding on kernels in the same manner as corn earworm, Helicoverpa zea. Unlike corn earworm, which tends to feed down through the silk before attacking the kernels at the tip of the ear, fall armyworm will feed by burrowing through the husk on the side of the ear. Cool, wet springs followed by warm, humid weather in the overwintering areas favor survival and reproduction of fall armyworm, allowing it to escape suppression by natural enemies. Once dispersal northward begins, the natural enemies are left behind. Therefore, although fall armyworm has many natural enemies, few act effectively enough to prevent crop injury.

Numerous species of parasitoids affect fall armyworm. The wasp parasitoids most frequently reared from larvae in the United States are Cotesia marginiventris (Cresson) and Chelonus texanus (Cresson) (both Hymenoptera: Braconidae), species that are also associated with other noctuid species. Among fly parasitoids, the most abundant is usually Archytas marmoratus (Townsend) (Diptera: Tachinidae). However, the dominant parasitoid often varies from place to place and from year to year. Luginbill (1928) and Vickery (Vickery R. A. (1929) Studies of the fall armyworm in the Gulf coast region of Texas. USDA Technical Bulletin 138. 63 pp.) describe and picture many of the fall armyworm parasitoids. The predators of fall armyworm are general predators that attack many other caterpillars. Among the predators noted as important are various ground beetles (Coleoptera: Carabidae); the striped earwig, Labidura riparia (Pallas) (Dermaptera: Labiduridae); the spined soldier bug, Podisus maculiventris (Say) (Hemiptera: Pentatomidae); and the insidious flower bug, Orius insidiosus (Say) (Hemiptera: Anthocoridae). Vertebrates such as birds, skunks, and rodents also consume larvae and pupae readily. Predation may be quite important, as Pair and Gross (1984) demonstrated 60 to 90 percent loss of pupae to predators in Georgia (Pair S. D. and Gross H. R. Jr. (1984) Field mortality of pupae of the fall armyworm, Spodoptera frupperda (J. E. Smith), by predators and a newly discovered parasitoid, Diapetimorpha introita. Journal of the Georgia Entomological Society 19: 22-26).

Numerous pathogens, including viruses, fungi, protozoa, nematodes, and a bacterium have been associated with fall armyworm (Gardner et al. 1984), but only a few cause epizootics. Among the most important are the S. frugiperda nuclear polyhedrosis virus (NPV), and the fungi Entomophaga aulicae, Nomuraea rileyi, and Erynia radicans. Despite causing high levels of mortality in some populations, disease typically appears too late to alleviate high levels of defoliation.

Sampling:

Moth populations can be sampled with blacklight traps and pheromone traps; the latter are more efficient. Pheromone traps should be suspended at canopy height, preferably in corn during the whorl stage. Catches are not necessarily good indicators of density, but indicate the presence of moths in an area. Once moths are detected it is advisable to search for eggs and larvae. A search of 20 plants in five locations, or 10 plants in 10 locations, is generally considered to be adequate to assess the proportion of plants infested. Sampling to determine larval density often requires large sample sizes, especially when larval densities are low or larvae are young, so it is not often used.

Insecticides:

Insecticides are usually applied to sweet corn in the southeastern states to protect against damage by fall armyworm, sometimes as frequently as daily during the silking stage. In Florida, fall armyworm is the most important pest of corn. It is often necessary to protect both the early vegetative stages and reproductive stage of corn. Because larvae feed deep in the whorl of young corn plants, a high volume of liquid insecticide may be required to obtain adequate penetration. Insecticides may be applied in the irrigation water if it is applied from overhead sprinklers. Granular insecticides are also applied over the young plants because the particles fall deep into the whorl. Some resistance to insecticides has been noted, with resistance varying regionally. Foster (1989) reported that keeping the plants free of larvae during the vegetative period reduced the number of sprays needed during the silking period (Foster R. E. (1989) Strategies for protecting sweet corn ears from damage by fall armyworms (Lepidoptera: Noctuidae) in southern Florida. Florida Entomologist 72: 146-151). The grower practice of concentrating the sprays at the beginning of the silking period instead of spacing the sprays evenly provided little benefit.

Cultural Techniques:

The most important cultural practice, employed widely in southern states, is early planting and/or early maturing varieties. Early harvest allows many corn ears to escape the higher armyworm densities that develop later in the season (Mitchell E. R. (1978) Relationship of planting date to damage by earworms in commercial sweet corn in north central Florida. Florida Entomologist 61: 251-255). Reduced tillage seems to have little effect on fall armyworm populations (All J. N. (1988) Fall armyworm (Lepidoptera: Noctuidae) infestations in no-tillage cropping systems. Florida Entomologist 71: 268-272), although delayed invasion by moths of fields with extensive crop residue has been observed, thus delaying and reducing the need for chemical suppression (Roberts P. M. and All J. N. (1993) Hazard for fall armyworm (Lepidoptera: Noctuidae) infestation of maize in double-cropping systems using sustainable agricultural practices. Florida Entomologist 76: 276-283).

Host Plant Resistance:

Partial resistance is present in some sweet corn varieties, but is inadequate for complete protection.

Biological Control:

Although several pathogens have been shown experimentally to reduce the abundance of fall armyworm larvae in corn, only Bacillus thuringiensis presently is feasible, and success depends on having the product on the foliage when the larvae first appear. Natural strains of Bacillus thuringiensis tend not to be very potent, and genetically modified strains improve performance (All J. N. et al. (1996) Controlling fall armyworm infestations in whorl stage corn with genetically modified Bacillus thuringiensis formulations. Florida Entomologist 79: 311-317).

### Spider Mites

Spider mites belong to the Acari (mite) family Tetranychidae, which includes about 1,200 species. They generally live on the undersides of leaves of plants and can cause damage by puncturing the plant cells to feed. Many species of spider mites may also spin protective silk webs to protect their colonies from predators. Spider mites are known to feed on several hundred species of plants.

Spider mites are less than 1 millimeter in size and vary in color. They lay small, spherical, initially transparent eggs which can be protected by silk webbing.

Hot, dry conditions are often associated with population build-up of spider mites. Under optimal conditions (approximately 27° C.), the two-spotted spider mite can hatch in as little as 3 days, and become sexually mature in as little as 5 days. One female can lay up to 20 eggs per day and can live for 2 to 4 weeks, laying hundreds of eggs. This accelerated reproductive rate allows spider mite populations to quickly develop resistance to pesticides, so chemical control methods can become ineffectual when the same pesticide is used over a prolonged period.

The best known member of the group is Tetranychus urticae, or the twospotted spider mite, which is dispersive and attacks a wide range of plants, including peppers, tomatoes, potatoes, beans, corn, cannabis, and strawberries. Dispersal of Tetranychus urticae is observed to be triggered by starvation, desiccation, wind and light, or in response to a heavily-infested plant (Li, J. and Margolies, D. C. (1994) Responses to direct and indirect selection on aerial dispersal behaviour in Tetranychus urticae. Heredity, 72: 10-22; Boykin, L. S. and Campbell, W. V. (1984) Wind Dispersal of the Twospotted Spider Mite (Acari: Tetranychidae) in North Carolina Peanut Fields. Environmental Entomology, 13(1): 221-227; Smitley, D. R. and Kennedy, G. G. (1985) Photo-oriented aerial-dispersal behavior of Tetranychus urticae (Acari: Tetranychidae) enhances escape from the leaf surface. Annals of the Entomological Society of America, 78(5): 609-614; Smitley, D. R. and Kennedy, G. G. (1988) Aerial dispersal of the two-spotted spider mite (Tetranychus urticae) from field corn. Experimental & Applied Acarology, 5(1): 33-46; Hussey, N. W. and Parr, W. J. (2011) Dispersal of the glasshouse red spider mite Tetranychus urticae Koch (Acarina, Tetranychidae). Entomologia Experimentalis et Applicata, 6(3): 207-214; Dicke, M. (1986) Volatile spider-mite pheromone and host-plant kairomone, involved in spaced-out gregariousness in the spider mite Tetranychus urticae. Physiological Entomology 11: 251-262).

Other species which can be important pests of commercial plants include Panonychus ulmi (fruit tree red spider mite) and Panonychus citri (citrus red mite).

### Sucking Pests

The three main taxonomic groups of sucking pests are: thrips (Thysanoptera), true bugs (Heteroptera [stink bugs, tarnished plant bugs, squash bugs]) and (spider) mites (Acarina). The sucking pests also include other Hemiptera like leaf/plant/tree hoppers, psyllids, aphids, whiteflies, mealybugs and scales. Sucking pests have piercing/sucking mouth parts to feed on sap. Some sucking insects inject toxic materials into the plant while feeding, and some transmit disease organisms. The southern green stink bug (Nezara viridula) and the neotropical brown stink bug (Euschistus heros) are two examples of very destructive sucking pests, especially in South American soybeans and other legumes grown in tropical and subtropical regions. The damage caused by E. heros when uncontrolled can get up to 30% on soybean (Vivan and Degrande (2011) Pragas da soja In: Boletim de pesquisa de soja (1^{st} ed., p. 297). Rondonopolis: Fundacao M T. (Boletim, 15)). Nezara viridula, however, is considered significantly more destructive, as it is more polyphagous and has a wider geographical range. Plants being attacked by sap-feeders will take on a shiny look and sticky feel. Plant symptoms include: plant distortion (leaf and stem twisting and curling, dead spots); excrement deposits (tar spots, honeydew and sooty mold); and foliage discoloration (spots and stipples, yellowing and bronzing).

The engineering of plants to express the insecticidal Bacillus thuringiensis (Bt) toxins have allowed effective control of lepidopteran pests such as the corn rootworm. However, phloem sap-sucking insects, such as aphids, whiteflies, planthoppers and plant bugs, have evolved from minor pests to major pests, because these is no Bt toxin with adequate insecticidal effects on these kinds of pests. Control of sucking insects with insecticides is not always effective. RNAi could be more effective against the adults of Pentatomidae pests (like N. viridula and E. heros) than with lepidopterans, due to their longer reproductive period. This extended adult period gives the introduced ds/siRNA time to influence the synthesis of new proteins and thus affect the behavior of the reproductive adults. While mating disruption might not be effective with pentatomids, an attract and RNAi-kill could be an effective way to control N. viridula and E. heros pest populations. Female E. heros are attracted to lures of methyl 2,6,10-trimethyltridecanoate (TMTD; Borges et al. (2001) Monitoring the Neotropical brown stink bug Euschistus heros (F.) (Hemiptera: Pentatomidae) with pheromone-baited traps in soybean fields. J. Appl. Entomol. 135). Females, males, and late-stage larvae of N. viridula are attracted to a male-produced pheromone, (Z)-α-bisabolene (17%), trans- and cis-1,2-epoxides of (Z)-α-bisabolene (44 and 15%, respectively), (E)-nerolidol (1.4%), and n-nonadecane (7.4%) (Aldrich et al. (2005) Pheromone strains of the Cosmopolitan pest, Nezara viridula (heteroptera: Pentatomidae) J. Exp. Zool 244(1)171-175). Food substrate at these lures can be treated with an RNAi to effect the mortality or reproductive behaviors of the attracted females. The use of pheromones and ds/siRNA specific to a particular species, ensures that there will be no non-target effects.

### Aphids

Aphids are soft-bodied insects that use their piercing sucking mouthparts to feed on plant sap. They usually occur in colonies on the undersides of tender terminal growth. Heavily-infested leaves can wilt or turn yellow because of excessive sap removal. While the plant may look bad, aphid feeding generally will not seriously harm healthy, established trees and shrubs.

However, some plants are very sensitive to feeding by certain aphid species. Saliva injected into plants by these aphids may cause leaves to pucker or to become severely distorted, even if only a few aphids are present. Also, aphid feeding on flower buds and fruit can cause malformed flowers or fruit.

Aphids produce large amounts of a sugary liquid waste called “honeydew”. The honeydew that drops from these insects can spot the windows and finish of cars parked under infested trees. A fungus called sooty mold can grow on honeydew deposits that accumulate on leaves and branches, turning them black. The appearance of sooty mold on plants may be the first time that an aphid infestation is noticed. The drops can attract other insects such as ants that will feed on the sticky deposits.

Some aphids are very important vectors of plant viruses. However, it is seldom possible to control these diseases by attempting to kill the aphid vectors with an insecticide. Aphids carrying viruses on their mouthparts may have to probe for only a few seconds or minutes before the plant is infected. Resistant varieties or sequential plantings may be helpful in reducing problems with some viruses that attack annual plants.

Infestations generally result from small numbers of winged aphids that fly to the plant and find it to be a suitable host. They deposit several wingless young on the most tender tissue before moving on to find a new plant. The immature aphids or nymphs that are left behind feed on plant sap and increase gradually in size. They mature in 7 to 10 days and then are ready to produce live young. Usually, all of them are females and each is capable of producing 40 to 60 offspring. The process is repeated several times, resulting in a tremendous population explosion. Less than a dozen aphid “colonizers” can produce hundreds to thousands of aphids on a plant in a few weeks. Aphid numbers can build until conditions are so crowded, or the plant is so stressed, that winged forms are produced. These winged forms fly off in search of new hosts and the process is repeated.

Early detection is the key to reducing aphid infestations. The flight of winged colonizers cannot be predicted, so weekly examination of plants will help to determine the need for control. The bud area and undersides of the new leaves are examined for clusters or colonies of small aphids. The presence of these colonies indicates that the aphids are established on the plants and their numbers will begin to increase rapidly. Small numbers of individual colonies on small plants can be crushed by hand or removed by pruning as they are found. In some cases, this may provide adequate control. If aphid colonies can be found on about 5% or more of foliage tips of a plant or planting, then a control measure should be considered. Most products used for aphid control work as contact insecticides. This means that the aphids must be hit directly with spray droplets so that they can be absorbed into the insect's body. Since aphids tend to remain on the lower leaf surface, they are protected by plant foliage. Thorough coverage, directed at growing points and protected areas, is important. It is difficult to treat large trees because of the high spray pressure necessary to penetrate the foliage and to reach the tallest portions of the tree. Hose-end sprayers can be used on 15 foot to 20 foot trees but they need to produce a stream rather than an even pattern to reach these levels. Skips in coverage are common and there is a significant potential for applicator exposure through drift and runoff Commercial applicators may have the necessary equipment but these treatments may be very expensive. Aphid control is rarely feasible in these situations.

Summer oils can be used against aphids on some types of trees and ornamental plantings. They kill by suffocating the insects and/or disrupting their membranes. The label has to be checked for cautions on sensitive plants; oils can injure the foliage of some plants. Weather conditions, especially high temperatures, can increase the potential for foliage burn. Dormant oils should not be sprayed during the growing season. There is no residual effect so additional applications may be necessary.

Fatty acid salts or insecticidal soaps are very good against aphids. As with summer oils, they apparently work to disrupt insect cell membranes. They require direct contact with the insects and leave no residual effect.

Nervous system insecticides, such as malathion, Dursban (chlorpyrifos), and Orthene (acephate), are labeled for use on many shade trees and ornamental plants for aphid control. As with oils and soaps, coverage is very important and a follow-up application may be necessary. The plant or crop that is being treated needs to be listed on the product label. Sevin (carbaryl) is not effective against many aphids so it is generally not a good choice for control unless recommended specifically. In fact, applications of Sevin may reduce the number of beneficial insects, such as lady beetles, and increase the potential for aphid outbreaks.

Plant-mediated RNAi gene knockdown can be an invaluable pest management tool. Ingestation of specific dsRNAi has been shown to significantly decrease the green peach aphid's (Myzus persicae) fecundity. Using Agrobacterium-mediate infiltration, Nicotiana benthaminana leaves can be made to express MpC002 and Rack-1 siRNAs. When M. persicae forage on these leaves the corresponding RNAi's in their salivary gland, and gut (respectively) are silenced and fecundity is reduced.

Aphid control is most valuable for new plantings, where excessive sap removal is more likely to affect general plant vigor. Established and otherwise healthy plants can tolerate moderate to heavy aphid infestations, although affected leaves may wilt and turn yellow and there may be some premature drop.

Good cultural practices, such as watering and fertilization, will help to reduce stress by these insects. Problems with honeydew and sooty mold may develop but tend to be temporary and disappear after the aphids are gone.

A few aphid species produce cupped or distorted leaves; these plants may lose some of their aesthetic appeal for the season. Once the distortion occurs, the leaves will remain cupped and twisted until they fall off. Usually, the infestation is not noticed until the injury has occurred. Insecticide applications often are less effective because the aphids are protected in the gnarled leaves.

Plants that become infected with an aphid-borne virus may be severely stunted and may die. Preventive sprays are rarely effective in keeping viruses out of plantings but they may reduce the spread within a group of susceptible plants.

Beneficial insects, such as lady beetles and lacewings, will begin to appear on plants with moderate to heavy aphid infestations. They may eat large numbers of aphids but the reproductive capability of aphids is so great that the impact of the natural enemies may not be enough to keep these insects at or below acceptable levels.

In some embodiments, the methods of the present disclosure can be used to control one or more pests listed in Table 4.

TABLE 4
List of exemplary pests which may be controlled by the methods of the
disclosure.

| Common Name | Genus Species | Family | Order |
|---|---|---|---|
| American dog | Dermacentor variabilis | Ixodidae | Acarina |
| tick | | | |
| Twospotted spider | Tetranychus urticae | Tetranychidae | Acarina |
| mite | | | |
| Cigarette beetle | Lasioderma serricorne | Anobiidae | Coleoptera |
| Azuki bean | Callosobruchus chinensis | Bruchidae | Coleoptera |
| weevil | | | |
| Spotted pine | Monochamus clamator | Cerambycidae | Coleoptera |
| sawyer | | | |
| Pine sawyer | Monochamus | Cerambycidae | Coleoptera |
| | galloprovincialis | | |
| Whitespotted | Monochamus scutellatus | Cerambycidae | Coleoptera |
| sawyer | | | |
| Black spruce | Tetropium castaneum | Cerambycidae | Coleoptera |
| beetle | | | |
| Brown spruce | Tetropium fuscum | Cerambycidae | Coleoptera |
| longhorn beetle | | | |
| Striped cucumber | Acalymma vittatum | Chrysomelidae | Coleoptera |
| beetle | | | |
| Northern corn | Diabrotica barberi | Chrysomelidae | Coleoptera |
| rootworm | | | |
| Western corn | Diabrotica | Chrysomelidae | Coleoptera |
| rootworm | undecimpunctata howardi | | |
| Western corn | Diabrotica virgifera | Chrysomelidae | Coleoptera |
| rootworm | virgifera | | |
| Cotton boll | Anthonomus grandis | Curculionidae | Coleoptera |
| weevil | | | |
| Banana weevil | Cosmopolites sordidus | Curculionidae | Coleoptera |
| Sweetpotato root | Cylas brunneus | Curculionidae | Coleoptera |
| borer | | | |
| Sweetpotato root | Cylas formicarius | Curculionidae | Coleoptera |
| borer | formicarius | | |
| Sweetpotato | Cylas formicarius | Curculionidae | Coleoptera |
| weevil | | | |
| African sweet | Cylas puncticollis | Curculionidae | Coleoptera |
| potato weevil | | | |
| West Indian | Metamasius hemipterus | Curculionidae | Coleoptera |
| sugarcane weevil | | | |
| New Guinea | Rhabdoscelus obscurus | Curculionidae | Coleoptera |
| sugarcane weevil | | | |
| Red palm weevil | Rhynchophorus | Curculionidae | Coleoptera |
| | ferrugineus | | |
| American palm | Rhynchophorus palmarum | Curculionidae | Coleoptera |
| weevil | | | |
| Carpet beetle | attagenus spp | Dermestidae | Coleoptera |
| Australian sap | Carpophilus davidsoni | Nitidulidae | Coleoptera |
| beetle | | | |
| Driedfruit beetle | Carpophilus hemipterus | Nitidulidae | Coleoptera |
| Flower beetle | Carpophilus mutilatus | Nitidulidae | Coleoptera |
| Soybean beetle | Anomala dubia | Scarabaeidae | Coleoptera |
| Soybean beetle | Anomala rufocuprea | Scarabaeidae | Coleoptera |
| Soybean beetle | Anomala schonfeldti | Scarabaeidae | Coleoptera |
| Soybean beetle | Anomala vitis | Scarabaeidae | Coleoptera |
| Grass grub beetle | Costelytra zealandica | Scarabaeidae | Coleoptera |
| Oriental beetle | Exomala orientalis | Scarabaeidae | Coleoptera |
| grub | | | |
| Yellowish | Heptophylla picea | Scarabaeidae | Coleoptera |
| elongate chafer | | | |
| | Hoplia equina | Scarabaeidae | Coleoptera |
| Date palm fruit | Oryctes elegans | Scarabaeidae | Coleoptera |
| stalk borer | | | |
| African | Oryctes monoceros | Scarabaeidae | Coleoptera |
| rhinoceros beetle | | | |
| Coconut | Oryctes rhinoceros | Scarabaeidae | Coleoptera |
| rhinoceros beetle | | | |
| Bracken chafer | Phyllopertha horticola | Scarabaeidae | Coleoptera |
| Melanesian | Scapanes australis | Scarabaeidae | Coleoptera |
| rhinoceros beetle | | | |
| White pine cone | Conophthorus coniperda | Scolytidae | Coleoptera |
| beetle beetle | | | |
| Ponderosa pine | Conophthorus ponderosae | Scolytidae | Coleoptera |
| cone beetle | | | |
| Red turpentine | Dendroctonus valens | Scolytidae | Coleoptera |
| beetle | | | |
| Western | Gnathotrichus retusus | Scolytidae | Coleoptera |
| pinewood stainer | | | |
| Western hemlock | Gnathotrichus sulcatus | Scolytidae | Coleoptera |
| wood stainer | | | |
| Mediterranean | Ips erosus | Scolytidae | Coleoptera |
| engraver beetle | | | |
| Pine engravers | Ips pini | Scolytidae | Coleoptera |
| Spruce bark | Ips typographus japonicus | Scolytidae | Coleoptera |
| beetle | | | |
| Spruce bark | Ips typographus | Scolytidae | Coleoptera |
| beetle | | | |
| | Pityogenes calcaratus | Scolytidae | Coleoptera |
| Six-spined spruce | Pityogenes chalcographus | Scolytidae | Coleoptera |
| bark beetle | | | |
| Smaller European | Scolytus multistriatus | Scolytidae | Coleoptera |
| elm bark beetle | | | |
| Large elm bark | Scolytus scolytus | Scolytidae | Coleoptera |
| beetle | | | |
| European | Trypodendron domesticus | Scolytidae | Coleoptera |
| hardwood | | | |
| ambrosia beetle | | | |
| Striped ambrosia | Trypodendron lineatum | Scolytidae | Coleoptera |
| beetle | | | |
| Black stem borer | Xylosandrus germanus | Scolytidae | Coleoptera |
| Flour beetles | Tribolium spp | Tenebrionidae | Coleoptera |
| Screwworm fly | Cochliomyia hominivorax | Calliphoridae | Diptera |
| Australian sheep | Lucilia cuprina | Calliphoridae | Diptera |
| blowfly | | | |
| Apple leaf midge | Dasineura mali | Cecidomyiidae | Diptera |
| Mosquitoe | Mansonia uniformis | Culicidae | Diptera |
| Tsetse fly | Glossina fuscipes fuscipes | Glossinidae | Diptera |
| Tsetse fly | Glossina morsitans | Glossinidae | Diptera |
| | submorsita | | |
| Tsetse fly | Glossina pallidipes | Glossinidae | Diptera |
| House fly | Musca domestica | Muscidae | Diptera |
| Mexican fruit fly | Anastrepha ludens | Tephritidae | Diptera |
| Melon fly | Bactrocera cucurbitae | Tephritidae | Diptera |
| Oriental fruit fly | Bactrocera dorsalis | Tephritidae | Diptera |
| Olive fruit fly | Bactrocera oleae | Tephritidae | Diptera |
| Peach fruit fly | Bactrocera zonatus | Tephritidae | Diptera |
| Mediterranean | Ceratitis capitata | Tephritidae | Diptera |
| fruit fly | | | |
| Cherry fruit fly | Rhagoletis cerasi | Tephritidae | Diptera |
| Apple maggot | Rhagoletis pomonella | Tephritidae | Diptera |
| Mullein bug | Campylomma verbasci | Miridae | Heteroptera |
| Western tarnished | Lygus hesperus | Miridae | Heteroptera |
| plant bug | | | |
| Rice leaf bug | Trigonotylus caelestialium | Miridae | Heteroptera |
| Neotropical | Euschistus heros | Pentatomidae | Heteroptera |
| Brown Stink Bug | | | |
| Southern green | Nezara viridula | Pentatomidae | Heteroptera |
| stinkbug | | | |
| Sunn pest | Eurygaster integriceps | Scutelleridae | Heteroptera |
| Greenhouse | Trialeurodes vaporariorum | Aleyrodidae | Homoptera |
| whitefly | | | |
| Melon aphid | Aphis gossypii | Aphididae | Homoptera |
| Rose apple aphid | Dysaphis plantaginea | Aphididae | Homoptera |
| California red | Aonidiella aurantii | Diaspididae | Homoptera |
| scale | | | |
| Oleander scale | Aspidiotus nerii | Diaspididae | Homoptera |
| San Jose scale | Quadraspidiotus | Diaspididae | Homoptera |
| | perniciosus | | |
| Maritime pine | Matsucoccus feytaudi | Margarodidae | Homoptera |
| scale | | | |
| Israeli pine bast | Matsucoccus josephi | Margarodidae | Homoptera |
| scale | | | |
| Vine mealybug | Planococcus ficus | Pseudococcidae | Homoptera |
| European pine | Neodiprion sertifer | Diprionidae | Hymenoptera |
| sawfly | | | |
| Oriental hornet | Vespa orientalis | Vespidae | Hymenoptera |
| Yellownecked | Kalotermes flavicollis | Kalotermitidae | Isoptera |
| dry-wood termite | | | |
| Subterranean | Heterotermes tenuis | Rhinotermitidae | Isoptera |
| termite | | | |
| Black-winged | Odontotermes formosanus | Rhinotermitidae | Isoptera |
| subterranean | | | |
| termite | | | |
| Leek moth | Acrolepiopsis assectella | Acrolepiidae | Lepidoptera |
| Apple fruit moth | Argyresthia conjugella | Argyresthiidae | Lepidoptera |
| Mulberry white | Rondotia menciana | Bombycidae | Lepidoptera |
| caterpillar | | | |
| Peach fruit moth | Carposina sasakii | Carposinidae | Lepidoptera |
| Chinese larch | Coleophora sinensis | Coleophoridae | Lepidoptera |
| casebearer | | | |
| European goat | Cossus cossus | Cossidae | Lepidoptera |
| moth | | | |
| Leopard moth | Zeuzera pyrina | Cossidae | Lepidoptera |
| Asiatic rice borer | Chilo suppressalis | Crambidae | Lepidoptera |
| Rice leaffolder | Cnaphalocrocis medinalis | Crambidae | Lepidoptera |
| moth | | | |
| Mexican rice | Eoreuma loftini | Crambidae | Lepidoptera |
| borer | | | |
| Eggplant borer | Leucinodes orbonalis | Crambidae | Lepidoptera |
| Asian corn borer | Ostrinia furnacalis | Crambidae | Lepidoptera |
| European corn | Ostrinia nubilalis | Crambidae | Lepidoptera |
| borer | | | |
| Jasmine moth | Palpita unionalis | Crambidae | Lepidoptera |
| Yellow stem | Scirpophaga incertulas | Crambidae | Lepidoptera |
| borer | | | |
| Peach twig borer | Anarsia lineatella | Gelechiidae | Lepidoptera |
| Tomato pinworm | Keiferia lycopersicella | Gelechiidae | Lepidoptera |
| Pink bollworm | Pectinophora gossypiella | Gelechiidae | Lepidoptera |
| Pink-spotted | Pectinophora scutigera | Gelechiidae | Lepidoptera |
| bollworm | | | |
| Potato | Phthorimaea operculella | Gelechiidae | Lepidoptera |
| tuberworm | | | |
| Angoumois grain | Sitotroga cerealella | Gelechiidae | Lepidoptera |
| moth | | | |
| Guatemalan | Tecia solanivora | Gelechiidae | Lepidoptera |
| potato tuber moth | | | |
| Tomato leafminer | Tuta absoluta | Gelechiidae | Lepidoptera |
| Japanese giant | Ascotis selenaria cretacea | Geometridae | Lepidoptera |
| looper | | | |
| Mulberry looper | Hemerophila atrilineata | Geometridae | Lepidoptera |
| Horse chestun | Cameraria ohridella | Gracillariidae | Lepidoptera |
| leafminer | | | |
| Citrus leaf miner | Phyllocnistis citrella | Gracillariidae | Lepidoptera |
| Apple leafminer | Phyllonorycter ringoniella | Gracillariidae | Lepidoptera |
| Forest tent | Malacosoma disstria | Lasiocampidae | Lepidoptera |
| caterpillar | | | |
| Pine tussock | Dasychira plagiata | Lymantriidae | Lepidoptera |
| moth | | | |
| Tea tussock moth | Euproctis pseudoconspersa | Lymantriidae | Lepidoptera |
| Gypsy moth | Lymantria dispar | Lymantriidae | Lepidoptera |
| Nun moth | Lymantria monacha | Lymantriidae | Lepidoptera |
| Indian gypsy | Lymantria obfuscata | Lymantriidae | Lepidoptera |
| moth | | | |
| Rusty tussock | Orgyia antiqua | Lymantriidae | Lepidoptera |
| moth | | | |
| Whitemarked | Orgyia leucostigma | Lymantriidae | Lepidoptera |
| tussock moth | | | |
| Douglas-fir | Orgyia pseudotsugata | Lymantriidae | Lepidoptera |
| tussock moth | | | |
| Black cutworm | agrotis ipsilon | Noctuidae | Lepidoptera |
| Turnip moth | Agrotis segetum | Noctuidae | Lepidoptera |
| Velvetbean | Anticarsia gemmatalis | Noctuidae | Lepidoptera |
| caterpillar | | | |
| Silver-Y moth | Autographa gamma | Noctuidae | Lepidoptera |
| Maize stalk borer | Busseola fusca | Noctuidae | Lepidoptera |
| Red bollworm | Diparopsis castanea | Noctuidae | Lepidoptera |
| Spiny bollworm | Earias insulana | Noctuidae | Lepidoptera |
| Spotted | Earias vittella | Noctuidae | Lepidoptera |
| bollworm | | | |
| Darksided | Euxoa messoria | Noctuidae | Lepidoptera |
| cutworm | | | |
| Redbacked | Euxoa ochrogaster | Noctuidae | Lepidoptera |
| cutworm | | | |
| Cotton bollworm | Helicoverpa armigera | Noctuidae | Lepidoptera |
| Oriental tobacco | Helicoverpa assulta | Noctuidae | Lepidoptera |
| budworm | | | |
| Corn earworm | Helicoverpa zea | Noctuidae | Lepidoptera |
| Flax budworm | Heliothis maritime adaucta | Noctuidae | Lepidoptera |
| Tobacco | Heliothis virescens | Noctuidae | Lepidoptera |
| budworm | | | |
| Cabbage moth | Mamestra brassicae | Noctuidae | Lepidoptera |
| Ear-cutting | Mythimna separata | Noctuidae | Lepidoptera |
| caterpillar | | | |
| Tomato looper | Plusia chalcites | Noctuidae | Lepidoptera |
| Soybean looper | Pseudoplusia includens | Noctuidae | Lepidoptera |
| Corn stalk borer | Sesamia nonagrioides | Noctuidae | Lepidoptera |
| Armyworm | Spodoptera cosmioides | Noctuidae | Lepidoptera |
| Southern | Spodoptera eridania | Noctuidae | Lepidoptera |
| armyworm | | | |
| Beet armyworm | Spodoptera exigua | Noctuidae | Lepidoptera |
| Fall armyworm | Spodoptera frugiperda | Noctuidae | Lepidoptera |
| Egyptian cotton | Spodoptera littoralis | Noctuidae | Lepidoptera |
| leafworm | | | |
| Tobacco | Spodoptera litura | Noctuidae | Lepidoptera |
| cutworm | | | |
| Yellow striped | Spodoptera ornithogalli | Noctuidae | Lepidoptera |
| armyworm | | | |
| Cabbage looper | Trichoplusia ni | Noctuidae | Lepidoptera |
| Cabbage looper | Trichoplusia oxygramma | Noctuidae | Lepidoptera |
| Diamondback | Plutella xylostella | Plutellidae | Lepidoptera |
| moth | | | |
| Citrus flower | Prays citri | Plutellidae | Lepidoptera |
| moth | | | |
| Olive moth | Prays oleae | Plutellidae | Lepidoptera |
| Bagworm moth | Thyridopteryx | Psychidae | Lepidoptera |
| | ephemeraeformis | | |
| Artichoke plume | Platyptilia carduidactyla | Pterophoridae | Lepidoptera |
| moth | | | |
| Navel | Amyelois transitella | Pyralidae | Lepidoptera |
| orangeworm | | | |
| Almond moth | Cadra cautella | Pyralidae | Lepidoptera |
| Honeydew moth | Cryptoblabes gnidiella | Pyralidae | Lepidoptera |
| Southern pine | Dioryctria amatella | Pyralidae | Lepidoptera |
| coneworm | | | |
| Webbing | Dioryctria disclusa | Pyralidae | Lepidoptera |
| coneworm | | | |
| Webbing | Dioryctria merkeli | Pyralidae | Lepidoptera |
| coneworm | | | |
| Carob moth | Ectomyelois ceratoniae | Pyralidae | Lepidoptera |
| Lesser cornstalk | Elasmopalpus lignosellus | Pyralidae | Lepidoptera |
| borer | | | |
| Warehouse moth | Ephestia elutella | Pyralidae | Lepidoptera |
| Raisin Moth | Ephestia figulilella | Pyralidae | Lepidoptera |
| Mediterranean | Ephestia kuehniella | Pyralidae | Lepidoptera |
| flour moth | | | |
| Olive pyralid | Euzophera pinguis | Pyralidae | Lepidoptera |
| moth | | | |
| Indian meal moth | Plodia interpunctella | Pyralidae | Lepidoptera |
| Clearwing borer | Ichneumonoptera | Sesiidae | Lepidoptera |
| | chrysophanes | | |
| Vine tree borer | Paranthrene regalis | Sesiidae | Lepidoptera |
| Dusky clearwing | Paranthrene tabaniformis | Sesiidae | Lepidoptera |
| Peachtree borer | Synanthedon exitiosa | Sesiidae | Lepidoptera |
| Apple clearwing | Synanthedon myopaeformis | Sesiidae | Lepidoptera |
| Lesser peachtree | Synanthedon pictipes | Sesiidae | Lepidoptera |
| borer | | | |
| Dogwood borer | Synanthedon scitula | Sesiidae | Lepidoptera |
| Currant clearwing | Synanthedon tipuliformis | Sesiidae | Lepidoptera |
| moth | | | |
| Grape rootborer | Vitacea polistiformis | Sesiidae | Lepidoptera |
| Pine | Thaumetopoea pityocampa | Thaumetopoeidae | Lepidoptera |
| processionary | | | |
| moth | | | |
| Cyprus | Thaumetopoea wilkinsoni | Thaumetopoeidae | Lepidoptera |
| processionary | | | |
| caterpillar | | | |
| Case-bearing | Tinea pellionella | Tineidae | Lepidoptera |
| clothes moth | | | |
| Webbing clothes | Tineola bisselliella | Tineidae | Lepidoptera |
| moth | | | |
| Smaller tea | Adoxophyes honmai | Tortricidae | Lepidoptera |
| tortrix | | | |
| Summerfruit | Adoxophyes orana fasciata | Tortricidae | Lepidoptera |
| tortrix | | | |
| Summerfruit | Adoxophyes orana | Tortricidae | Lepidoptera |
| tortrix | | | |
| Apple peel | Adoxophyes reticulana | Tortricidae | Lepidoptera |
| tortricid | | | |
| Fruittree | Archips argyrospila | Tortricidae | Lepidoptera |
| leafroller | | | |
| Asiatic leafroller | Archips breviplicanus | Tortricidae | Lepidoptera |
| Apple tortrix | Archips fuscocupreanus | Tortricidae | Lepidoptera |
| Fruittree tortrix | Archips podana | Tortricidae | Lepidoptera |
| Rose tortrix moth | Archips rosana | Tortricidae | Lepidoptera |
| Orange tortrix | Argyrotaenia citrana | Tortricidae | Lepidoptera |
| Redbanded | Argyrotaenia velutinana | Tortricidae | Lepidoptera |
| leafroller | | | |
| European | Cacoecimorpha pronubana | Tortricidae | Lepidoptera |
| carnation tortrix | | | |
| Eastern spruce | Choristoneura fumiferana | Tortricidae | Lepidoptera |
| budworm | | | |
| Obliquebanded | Choristoneura rosaceana | Tortricidae | Lepidoptera |
| leafroller | | | |
| False codling | Cryptophlebia leucotreta | Tortricidae | Lepidoptera |
| moth | | | |
| Brownheaded | Ctenopseustis herana | Tortricidae | Lepidoptera |
| leafroller | | | |
| Brownheaded | Ctenopseustis obliquana | Tortricidae | Lepidoptera |
| leafroller | | | |
| Beech moth | Cydia fagiglandana | Tortricidae | Lepidoptera |
| Pea moth | Cydia nigricana | Tortricidae | Lepidoptera |
| Codling moth | Cydia pomonella | Tortricidae | Lepidoptera |
| Chestnut tortrix | Cydia splendana | Tortricidae | Lepidoptera |
| Spruce cone | Cydia strobilella | Tortricidae | Lepidoptera |
| moth | | | |
| Chinese tortrix | Cydia trasias | Tortricidae | Lepidoptera |
| Cherrybark tortrix | Enarmonia formosana | Tortricidae | Lepidoptera |
| moth | | | |
| Grape berry moth | Endopiza viteana | Tortricidae | Lepidoptera |
| South African | Epichoristodes acerbella | Tortricidae | Lepidoptera |
| carnation tortrix | | | |
| Lightbrown apple | Epiphyas postvittana | Tortricidae | Lepidoptera |
| moth | | | |
| Suma leaftier | Episimus argutanus | Tortricidae | Lepidoptera |
| moth | | | |
| Carambola fruit | Eucosma notanthes | Tortricidae | Lepidoptera |
| borer | | | |
| Western pine | Eucosma sonomana | Tortricidae | Lepidoptera |
| shootborer | | | |
| European grape | Eupoecilia ambiguella | Tortricidae | Lepidoptera |
| berry moth | | | |
| Plum fruit moth | Grapholita funebrana | Tortricidae | Lepidoptera |
| Oriental fruit | Grapholita molesta | Tortricidae | Lepidoptera |
| moth | | | |
| Lesser | Grapholita prunivora | Tortricidae | Lepidoptera |
| appleworm | | | |
| Oriental tea | Homona magnanima | Tortricidae | Lepidoptera |
| tortrix moth | | | |
| European | Lobesia botrana | Tortricidae | Lepidoptera |
| grapevine moth | | | |
| Fruitlet mining | Pammene rhediella | Tortricidae | Lepidoptera |
| tortrix | | | |
| Dark oblique- | Pandemis heparana | Tortricidae | Lepidoptera |
| barred twist | | | |
| Threelined | Pandemis limitata | Tortricidae | Lepidoptera |
| leafroller | | | |
| Apple pandemis | Pandemis pyrusana | Tortricidae | Lepidoptera |
| Greenheaded | Planotortrix octo | Tortricidae | Lepidoptera |
| leafroller | | | |
| Variegated | Platynota flavedana | Tortricidae | Lepidoptera |
| leafroller | | | |
| Tufted apple | Platynota idaeusalis | Tortricidae | Lepidoptera |
| budmoth | | | |
| Omnivorous | Platynota stultana | Tortricidae | Lepidoptera |
| leafroller | | | |
| Blackheaded | Rhopobota naevana | Tortricidae | Lepidoptera |
| fireworm | | | |
| European pine | Rhyacionia buoliana | Tortricidae | Lepidoptera |
| shoot moth | | | |
| Nantucket pine tip | Rhyacionia frustrana | Tortricidae | Lepidoptera |
| moth | | | |
| Pitch pine tip | Rhyacionia rigidana | Tortricidae | Lepidoptera |
| moth | | | |
| Ponderosa pine | Rhyacionia zozana | Tortricidae | Lepidoptera |
| tip moth | | | |
| Blueberry | Sparganothis sulfureana | Tortricidae | Lepidoptera |
| leafroller | | | |
| Eye-spotted | Spilonota ocellana | Tortricidae | Lepidoptera |
| budmoth | | | |
| Larch budmoth | Zeiraphera diniana | Tortricidae | Lepidoptera |
| Microworm | Panagrellus redivivus | Panagrolaimidae | Rhabditida |
| Onion thrips | Thrips tabaci | Thripidae | Thysanoptera |

### EXAMPLES

The following examples are given for the purpose of illustrating various embodiments of the disclosure and are not meant to limit the present invention in any fashion. Changes therein and other uses which are encompassed within the spirit of the invention, as defined by the scope of the claims, will occur to those skilled in the art. Throughout the below Examples, various references to pheromone compounds are made. For ease of reference the following abbreviations and corresponding compounds are provided:
Z11-16:Ald; (Z)-11-Hexadecenal, CAS #53939-28-9
Z9-16:Ald; (Z)-9-Hexadecenal, CAS #56219-04-6
Z11-16:OH; (Z)-11-Hexadecen-1-ol, CAS #56683-54-6
Z11-16:Ac; (Z)-11-Hexadecenyl acetate, CAS #34010-21-4
16:Ald; Hexadecanal, CAS #629-80-1
Z9-14:Ald; (Z)-9-Tetradecenal, CAS #53939-27-8
Z9-14:Ac; (Z)-9-Tetradecenyl acetate
Z9E12-14:Ac; (Z,E)-9,12-Tetradecadienyl acetate
MCA: 4-methoxycinnamaldehyde
TMTD: methyl 2,6,10-trimethyltridecanoate

### Example 1: Euschistus heros Damage Control in Soybean Achieved by “Attract and (RNAi−) Kill” of Females

Objective:

Demonstrate improvements in the damage control achieved through the attraction of females, via the TMTD pheromone, to traps baited with dsRNA food sources. Females that ingest the baited food consume dsRNA that target chromatin-remodeling ATPase transcripts, brahma, mi-2, and iswi (Fishilevich et al. (2016) Use of chromatin remodeling ATPases as RNAi targets for parental control of western corn rootworm (Diabrotica virgifera virgifera) and Neotropical brown stink bug (Euschistus heros). Insect biochemistry and molecular biology, 71: 58-71). These dsRNAs strongly reduce fecundity in the exposed female, and possibly even the next generation.

Materials and Methods:

There are three square field plots of equal size, each 12 ha, separated by at least 200 m, for attract and RNAi-kill (RNAi), traditional attract and insecticide-kill (A&K), and an Untreated Control (UTC).

Inputs:

Each plot is planted with soybean. Fertilization, disease control, none experiment target pest control, and weed control is done as per protocol for a high yield soybean farmer. Pheromone (methyl 2,6,10-trimethyltridecanoate [TMTD]) is formulated onto rubber septum, sheltered within multiple traps containing a food source. The food source is treated with a combination of 5 dsRNAs (brahma, mi-2, iswi-1, iswi-2, chd1).

Experimental:

Field plot 1, RNAi=attract and RNAi-kill at multiple traps; Field plot 2, A&K=attract and kill with traditional pentatomid insecticides at multiple traps; Field plot 3, UTC=no treatment.
a. Both RNAi and insecticide traps are periodically monitored; specimen within the traps are identified by species and counted. Routine oviposition and larva count transects are conducted following daily trap counts of >10 E. heros individuals. Damage to the vegetative and reproductive stages of the soybean crop are measured by harvesting samples and rating foliar damage and counting the number of damaged soybeans.

### Example 2: Combined Control of N. viridula with RNAi and Spodoptera Cosmioides with Mating Disruption in Soybeans

Objective:

Demonstrate improvements in the damage control of soybeans, by controlling two key pests via different modes of action. N. viridula is controlled with attract and kill-type control, using the male pheromone blend ((Z)-α-bisabolene, trans- and cis-1,2-epoxides of (Z)-α-bisabolene, (E)-nerolidol, and n-nonadecane) at traps containing dsRNA-baited food sources. S. cosmiodes is controlled with mating disruption using a sprayable formulation of Spodoptera latifascia's sex pheromone (Z9-14Ac & Z9E12-14Ac). Spodoptera latisfascia's pheromones have been shown to be effective at attracting and monitoring populations of S. cosmiodes in Brazil (Silvie, Pierre, and Jean-François Silvain IRD. Spodoptera frupperda and other species captured in pheromone traps in cotton cropping systems (Mato Grosso State, Brazil). Proceedings of the 5^{th} Brazilian Congress of Cotton). Females, males, and late-stage N. viridula larvae can be attracted to the pheromones emitted from the feeding traps, where they may consume dsRNA that targets chromatin-remodeling ATPase transcripts, brahma, mi-2, and iswi. These dsRNAs strongly reduce fecundity and molting.

Materials and Methods:

There are four square field plots of equal size, each 12 ha, separated by at least 200 m, for attract and kill alone (RNAi), mating disruption alone (MD), attract and kill combined with mating disruption (RNAi-MD), and an Untreated Control (UTC).

Inputs:

Each plot is planted with soybean. Fertilization, disease control, none experiment target pest control, and weed control is done as per protocol for a high yield soybean farmer. Pheromone ((Z)-α-bisabolene (17%), trans- and cis-1,2-epoxides of (Z)-α-bisabolene (44 and 15%, respectively), (E)-nerolidol (1.4%), and n-nonadecane (7.4%)) is formulated onto rubber septum, sheltered within multiple traps containing a food source. The food source is treated with a combination of 5 dsRNAs (brahma, mi-2, chd1). A sprayable formulation of Z9-14Ac and Z9E12-14Ac is applied to the mating disruption plots.

Experimental:

Field plot 1, RNAi=attract and RNAi-kill at multiple traps; Field plot 2, MD=sprayable pheromone AI; Field plot 3, RNAi-MD=sprayable pheromone AI, attract and RNAi-kill at multiple taps; Field plot 4, UTC=no treatment.

Mating disruption is monitored by 4 universal bucket traps for S. cosmiodes, positioned as a square with 30 m inter-trap distances, in the center of each plot. Trap counts of adult moths is obtained beginning the day after the first pheromone treatment (canopy closure). Routine oviposition and larva count transects is conducted following daily trap counts of >10 specimen of either S. cosmiodes males from the UTC, or N. viridula individuals from the feeding traps. Damage to the vegetative and reproductive stage of the soybean crop is measured by harvesting samples and rating foliar damage and counting the number of damaged soybeans.

### Example 3: Helicoverpa armigera Damage Control in Corn Achieved by Augmenting Mating Disruption with Larvicide RNAi

Objective:

Demonstrate improvements in damage control when combining RNAi with mating disruption strategies.

Materials and Methods:

There are 3 square field plots of equal size, each 12 ha, separated by at least 200 m, for mating disruption (MD), MD with RNAi treatments, and an Untreated Control (UTC).

Inputs:

Each plot has a corn hybrid, either conventional or round-up ready, but not Bt, 115-118 RM. Fertilization, none experiment target pest control, and weed control is done as per protocol for a high yield corn farmer. Pheromone is formulated as a sprayable emulsion concentrate (Z11-16Ald 97%; Z9-16Ald 3%). siRNA (single interfering RNA) is sprayed on corn ears via drop sprayers. Potential hormone biosynthesis genes to target with siRNA are the prothoraciotropic hormone (AY286543.1/AY780527.1), Molt-regulating transcription factors3 (AF337637.3/FJ009448.1), or the Eclosion hormone precursor (AY822476.1) (Choudhary, M. and Sahi, S. (2011) In silico designing of insecticidal small interfering RNA (siRNA) for Helicoverpa armigera control. Indian Journal of Experimental Biology, 49(6): 469-474).

Experimental:

Field plot 1, MD=sprayable pheromone AI; Field plot 2, MD & RNAi=sprayable pheromone AI, sprayable siRNA; Field plot 3, UTC=no treatment.

Mating disruption is monitored by 4 Heliothis traps for H. armigera, positioned as a square with 30 m inter-trap distances, in the center of each plot. Trap counts of adult moths is obtained beginning the day after the first pheromone treatment (canopy closure). Routine larva count transects is conducted following daily trap counts of >10 males from the UTC. Larva instar stage is recorded, and a sample of larva is marked and transferred to a caged plant where their growth rate can be recorded. Damage to the grain on the ears is measured by harvesting a sample of ears and counting the number of ears with damage and the percent of the ear that was damaged.

### Example 4: Diabrotica virgifera virgifera Damage Control in Corn Achieved by Augmenting Mating Disruption with a Transgenic Corn Hybrid Expressing dsRNA that Leads to Larval Mortality

Objective:

Demonstrate improvements in damage control when combining dsRNA-expressing crops with mating disruption strategies.

Materials and Methods:

There are 3 square field plots of equal size, each 12 ha, separated by at least 200 m, for mating disruption (MD), MD with RNAi treatments, and an Untreated Control (UTC). All plantings are rain-fed, no irrigation.

Inputs:

One (MD & RNAi) treatment plot is planted with a V-ATPase A subunit 2 dsRNA-expressing transgenic corn line (Baum et al. (2007) Control of coleopteran insect pests through RNA interference. Nature Biotechnology 25: 1322-1326). The dsRNA silence the genes encoding a vacuolar-type ft-ATPase and leads to larva mortality. The MD and UTC plots are planted with a conventional corn hybrid.

Experimental:

Field plot 1, MD=scattered 4-methoxycinnamaldehyde (MCA)-coated corn granules (‘grits’); Field plot 2, MD & RNAi=scattered grits, dsRNA-expressing cotton; Field plot 3, UTC=no treatment.

Mating disruption is monitored by 4 pheromone traps for D. virgifera, positioned as a square with 30 m inter-trap distances, in the center of each plot. Trap counts of adult moths are obtained beginning the day after the first pheromone treatment (canopy closure). Damage to the crop assessed by removing a sample of plants and quantifying root mass.

### Example 5: Spodoptera frugiperda Damage Control in Corn Achieved by Augmenting Mating Disruption with Reducing Females' Oviposition Rates with the Ingestion of dsRNA Silencing Genes for Juvenile Hormone Production

Objective:

Demonstrate improvements in the damage control achieved through mating disruption by significantly lowering oviposition of those females that mate; with the inclusion of dsRNA-infused sucrose into pheromone mating disruption strategies. Key variables: Point source (feeding station) application of phagostimulant or broadcast spray, inclusion of floral volatiles into phagostimulant.

Materials and Methods:

There are four square field plots of equal size, each 12 ha, separated by at least 200 m, for mating disruption (MD), MD with dsRNA-infused phagostimulants via point sources (PS-MD) or broadcast spraying (BS-MD), and an Untreated Control (UTC).

Inputs:

Each plot has a corn hybrid, either conventional or round-up ready, but not Bt, 115-118 RM. Fertilization, none experiment target pest control, and weed control are done as per protocol for a high yield corn farmer. Pheromone is formulated as a sprayable emulsion concentrate (Z9-14Ac 87%, Z11-16Ac 13%). Phagostimulant is formulated for both broadcast sprays and feeding stations (modified centrifuge tube reservoir); aqueous solution of (5%) sucrose with two known S. frugiperda dsRNAs (293-570 nucleotide allatostatin-C-type-sequence [AS-C-dsRNA], and 23-218 nucleotide allatotropin 2 sequence [AT 2-dsRNA](Griebler, M. et al. (2008) RNA interference with the allatoregulating neuropeptide genes from the fall armyworm Spodoptera frugiperda and its effects on the JH titer in the hemolymph. Journal of Insect Physiology 54: 997-1007).

Experimental:

Field plot 1, MD=sprayable pheromone AI (Z9-14Ac 87%, Z11-16Ac 13%); Field plot 2, PS-MD=sprayable pheromone AI, multiple feeding stations; Field plot 3, BS-MD=sprayable pheromone AI, sprayable phagostimulant; Field 4, UTC=no treatment.

Mating disruption is monitored by 4 universal bucket traps positioned as a square, with 30 m inter-trap distances, in the center of each plot. Trap counts of adult moths are obtained beginning the day after the first pheromone (and phagostimulant) treatment (canopy closure). Routine oviposition and larva count transects are conducted following daily trap counts of >10 males from the UTC. Damage to the grain on the ears is measured by harvesting a sample of ears and counting the number of ears with damage and the percent of the ear that was damaged.

### Example 6: Helicoverpa armigera/Zea Damage Control in Cotton Achieved by Augmenting Mating Disruption with a Transgenic Cotton Hybrid Expressing dsRNA that Leads to the RNAi of Gossypol Detoxifying Genes in Larva

Objective:

Demonstrate improvements in damage control when combining dsRNA-expressing crops with mating disruption strategies.

Materials and Methods:

There are 3 square field plots of equal size, each 12 ha, separated by at least 200 m, for mating disruption (MD), MD with RNAi treatments, and an Untreated Control (UTC).

Inputs:

One plot is planted with a GST1/CYP6AE14 dsRNA-expressing cotton hybrid (treatment) (Mao, Y. B. et al. (2007) Silencing a cotton bollworm P450 monooxygenase gene by plant-mediated RNAi impairs larval tolerance of gossypol. Nature Biotechnology 25: 1307-1313), while the rest are planted with a transgenic cotton containing dsGFP (a harmless green fluorescent protein, control). The hairpin dsRNA, in the treatment plot, silence the genes encoding a glutathione-S-transferase, and cytochrome P450 monooxygenase respectively. Fertilization, none experiment target pest control, disease control, growth regulators, and weed control are done as per protocol for a high yield cotton farmer.

Experimental:

Field plot 1, MD=sprayable pheromone AI (Z11-16Ald 97%; Z9-16Ald 3%), dsGFP-expressing cotton; Field plot 2, MD & RNAi=sprayable pheromone AI (Z11-16Ald 97%; Z9-16Ald 3%), dsGST1/CYRP6AE13-expressing cotton; Field plot 3, UTC=dsGFP-expressing cotton.

Mating disruption is monitored by 4 Heliothis traps for H. armigera/zea, positioned as a square with 30 m inter-trap distances, in the center of each plot. Trap counts of adult moths is obtained beginning the day after the first pheromone treatment (canopy closure). Routine larva count transects is conducted following daily trap counts of >10 males from the UTC. Larva instar stage is recorded, and a sample of larva is marked and transferred to a caged plant where their growth rate can be recorded. Damage to the grain on the ears is measured by harvesting a sample of ears and counting the number of ears with damage and the percent of the ear that was damaged.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

### INCORPORATION BY REFERENCE

All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

What is claimed is:

1. A method of reducing or preventing plant damage in a field plot containing a plant population and one or more pests capable of damaging the plants, said method comprising:
a) applying a mating disruption tactic to the field plot, which is capable of disrupting the mating of the one or more pests; and
b) disrupting expression of one or more target genes by RNA interference (RNAi) in the one or more pests;
wherein said method reduces or prevents plant damage from the one or more pests as a result of the applied mating disruption tactic and disrupted target gene expression, when compared to a control field plot.

2. The method of claim 1, wherein the one or more pests comprises one or more sucking pests.

3. The method of claim 1, wherein the one or more pests is a member of the class Insecta.

4. The method of claim 1, wherein the one or more pests is a member of the order Lepidoptera.

5. The method of claim 1, wherein the one or more pests is a member of the order Hemiptera.

6. The method of claim 1, wherein the one or more pests is a member of the family Noctuidae.

7. The method of claim 1, wherein the one or more pests is a member of the family Pentatomidae.

8. The method of claim 1, wherein the one or more pests is a member of the order Coleoptera.

9. The method of claim 1, wherein the one or more pests is a member of the family Curculionidae.

10. The method of claim 1, wherein the one or more pests is a member of a genus selected from the group consisting of: Helicoverpa, Spodoptera, Euschistus, Anthonomus and Nezara, or any combination thereof.

11. The method of claim 1, wherein the one or more pests is a species selected from the group consisting of: Helicoverpa zea, Helicoverpa armigera, Spodoptera frugiperda, Spodoptera cosmioides, Euschistus heros, Anthonomus grandis and Nezara viridula, or any combination thereof.

12. The method of claim 1, wherein the target gene comprises one or more pheromone biosynthesis-activating neuropeptides (PBANs).

13. The method of claim 1, wherein the target gene comprises: chromatin-remodeling ATPases, prothoraciotropic hormone, molt-regulating transcription factors 3, eclosion hormone precursor, p450 monooxygenase, allatoregulating neuropeptides, 3-hydroxy-3-methylglutaryl coenzyme A reductase (HMGR), vacuolar-type H⁺-ATPases, chitinases, PCGP, arf1, arf2, tubulins, cullin-1, acetylcholine esterases, β1 integrins, iron-sulfur proteins, aminopeptidaseN, arginine kinases, chitin synthases, or any combination thereof, in the one or more pests.

14. The method of claim 1, wherein applying a mating disruption tactic comprises applying one or more pheromones or pheromone blends.

15. The method of claim 14, wherein the one or more pheromones or pheromone blends comprises one or more pheromones listed in Table 2.

16. The method of claim 14, wherein the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

17. The method of claim 1, wherein the RNAi comprises one or more double-stranded RNA, one or more small interfering RNA (siRNA), or a combination thereof.

18. The method of claim 17, wherein the one or more double-stranded RNA, one or more small interfering RNA (siRNA), or a combination thereof, are expressed in a plant.

19. The method of claim 17, wherein the one or more double-stranded RNA, one or more small interfering RNA (siRNA), or a combination thereof, are formulated for a broadcast spray, a feeding station, a food trap, or any combination thereof.

20. The method of claim 1, wherein the mating disruption tactic controls one type of pest and disrupting the expression of one or more target genes by RNAi controls another type of pest.

21. A method of reducing or preventing plant damage in a field plot containing a plant population and one or more pests capable of damaging the plants, said method comprising applying an attract-and-kill tactic to the field plot comprising:
a) applying one or more semiochemicals or factors; and
b) disrupting expression of one or more target genes by RNA interference (RNAi) in the one or more pests,
wherein said method reduces or prevents plant damage from the one or more pests as a result of the applied attract-and-kill tactic when compared to a control field plot.

22. The method of claim 21, wherein the one or more pests comprises one or more sucking pests.

23. The method of claim 21, wherein the one or more pests is a member of the class Insecta.

24. The method of claim 21, wherein the one or more pests is a member of the order Lepidoptera.

25. The method of claim 21, wherein the one or more pests is a member of the order Hemiptera.

26. The method of claim 21, wherein the one or more pests is a member of the family Noctuidae.

27. The method of claim 21, wherein the one or more pests is a member of the family Pentatomidae.

28. The method of claim 21, wherein the one or more pests is a member of the order Coleoptera.

29. The method of claim 21, wherein the one or more pests is a member of the family Curculionidae.

30. The method of claim 21, wherein the one or more pests is a member of a genus selected from the group consisting of: Helicoverpa, Spodoptera, Euschistus, Anthonomus and Nezara, or any combination thereof.

31. The method of claim 21, wherein the one or more pests is a species selected from the group consisting of Helicoverpa zea, Helicoverpa armigera, Spodoptera frugiperda, Spodoptera cosmioides, Euschistus heros, Anthonomus grandis and Nezara viridula, or any combination thereof.

32. The method of claim 21, wherein the target gene comprises one or more pheromone biosynthesis-activating neuropeptides (PBANs).

33. The method of claim 21, wherein the target gene comprises: chromatin-remodeling ATPases, prothoraciotropic hormone, molt-regulating transcription factors 3, eclosion hormone precursor, 050 monooxygenase, allatoregulating neuropeptides, 3-hydroxy-3-methylglutaryl coenzyme A reductase (HMGR), vacuolar-type H⁺-ATPases, chitinases, PCGP, arf1, arf2, tubulins, cullin-1, acetylcholine esterases, 1 integrins, iron-sulfur proteins, aminopeptidaseN, arginine kinases, chitin synthases, or any combination thereof, in the one or more pests.

34. The method of claim 21, wherein the one or more semiochemicals comprise one or more attractants.

35. The method of claim 34, wherein the one or more attractants comprise one or more pheromones or pheromone blends.

36. The method of claim 35, wherein the one or more pheromones or pheromone blends comprises one or more pheromones listed in Table 2.

37. The method of claim 35, wherein the one or more pheromones or pheromone blends comprise methyl 2,6,10-trimethyltridecanoate (TMTD), (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

38. The method of claim 21, wherein the RNAi comprises one or more double-stranded RNA, one or more small interfering RNA (siRNA), or a combination thereof.

39. The method of claim 21, wherein the one or more double-stranded RNA, one or more small interfering RNA (siRNA), or a combination thereof, are expressed in a plant.

40. The method of claim 21, wherein the one or more double-stranded RNA, one or more small interfering RNA (siRNA), or a combination thereof, are formulated for a broadcast spray, a feeding station, a food trap, or any combination thereof.

41. A method of reducing or preventing plant damage in a field plot as substantially described herein.

42. The method of claim 1, wherein applying a mating disruption tactic comprises spraying one or more pheromones or pheromone blends in the field plot.

43. The method of claim 42, wherein the one or more pheromones or pheromone blends comprises one or more pheromones listed in Table 2.

44. The method of claim 42, wherein the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-1-hexadecenal, (Z)-9-hexadecenal, (Z)-11-hexadecenyl acetate, 4-methoxycinnamaldehyde, or any combination thereof.

45. The method of claim 1, wherein applying a mating disruption tactic comprises emitting one or more pheromones or pheromone blends from one or more dispensers placed in the field plot.

46. The method of claim 45, wherein the one or more pheromones or pheromone blends comprises one or more pheromones listed in Table 2.

47. The method of claim 45, wherein the one or more pheromones or pheromone blends comprises: methyl 2,6,10-trimethyltridecanoate, (Z)-α-bisabolene, trans- and cis-1,2-epoxides of (Z)-α-bisabolene, (E)-nerolidol, n-nonadecane, (Z)-9-tetradecenyl acetate, (Z,E)-9,12-tetradecadienyl acetate, (Z)-11-hexadecenal, (Z)-9-hexadecenal, (Z)-11-hexadecenyl acetate, 4-methoxycinnamaldehyde, or any combination thereof.

48. The method of claim 1, wherein applying a mating disruption tactic comprises spraying one or more pheromones or pheromone blends in the field plot, and wherein disrupting expression of one or more target genes comprises feeding dsRNA to the one or more pests.

49. The method of claim 48, wherein the dsRNA fed to the one or more pests are infused in phagostimulants.

50. The method of claim 1, wherein applying a mating disruption tactic comprises spraying one or more pheromones or pheromone blends in the field plot, and wherein disrupting expression of one or more target genes comprises spraying RNAi molecules in the field plot.

51. The method of claim 50, wherein the RNAi molecules are siRNA or dsRNA infused in phagostimulants.

52. The method of claim 1, wherein applying a mating disruption tactic comprises scattering pheromone- or pheromone blend-coated granules in the field plot, and wherein disrupting expression of one or more target genes comprises growing transgenic plants expressing RNAi molecules in the field plot as a source of food for the one or more pests.

53. The method of claim 1, wherein applying a mating disruption tactic comprises spraying one or more pheromones or pheromone blends in the field plot, and wherein disrupting expression of one or more target genes comprises growing transgenic plants expressing RNAi molecules in the field plot as a source of food for the one or more pests.

54. The method of claim 21, wherein applying one or more semiochemicals or factors comprises emitting one or more pheromones or pheromone blends from one or more dispensers placed in one or more traps in the field plot.

55. The method of claim 21, wherein applying one or more semiochemicals or factors comprises emitting one or more pheromones or pheromone blends from one or more dispensers placed in one or more traps in the field plot, and wherein disrupting expression of one or more target genes comprises feeding dsRNA to the one or more pests.
